SEQUENCE LISTING (61 sequences)

SEQ ID NO: 1 - protein, 11 aa, Artificial Sequence
  Synthetic residues 47-57 of HIV-1 TAT
YGRKKRRQRRR

SEQ ID NO: 2 - RNA, 33 nt, Artificial Sequence
  Synthetic RNA 1
gagaacgcaccacuuuacgaguuuuagagcuag

SEQ ID NO: 3 - RNA, 39 nt, Artificial Sequence
  Synthetic RNA 2
  modified_base (1)..(1): phosphate
aaauagcaaguuaaaauaaggcuaguccguuaucaacuu

SEQ ID NO: 4 - RNA, 28 nt, Artificial Sequence
  Synthetic RNA 3
  modified_base (1)..(1): phosphate
gaaaaaguggcaccgagucggugcuuuu

SEQ ID NO: 5 - DNA, 36 bp, Artificial Sequence
  Synthetic Splint 1->2
ccttattttaacttgctatttctagctctaaaactc

SEQ ID NO: 6 - DNA, 48 bp, Artificial Sequence
  Synthetic Splint 2->3
aaaagcaccgactcggtgccactttttcaagttgataacggactagcc

SEQ ID NO: 7 - RNA, 33 nt, Artificial Sequence; identical to SEQ ID NO: 2
  Synthetic RNA 1
  modified_base (1)..(1): 2′-O-methyl
  modified_base (2)..(4): 2′-O-methyl with phosphorothioate
  modified_base (29)..(32): 2′-O-methyl

SEQ ID NO: 8 - RNA, 39 nt, Artificial Sequence; identical to SEQ ID NO: 3
  Synthetic RNA 2m1
  modified_base (1)..(1): phosphate
  modified_base (4)..(7): 2′-O-methyl
  modified_base (35)..(39): 2′-O-methyl

SEQ ID NO: 9 - RNA, 39 nt, Artificial Sequence; identical to SEQ ID NO: 3
  Synthetic RNA 2m2
  modified_base (1)..(1): phosphate
  modified_base (4)..(7): 2′-O-methyl
  modified_base (35)..(37): 2′-O-methyl

SEQ ID NO: 10 - RNA, 28 nt, Artificial Sequence
  Synthetic RNA 3
  modified_base (1)..(1): phosphate
  modified_base (5)..(25): 2′-O-methyl
  modified_base (26)..(28): 2′-O-methyl with phosphorothioate
gaaaaaguggcaccgagucggugcuuuu

SEQ ID NO: 11 - RNA, 33 nt, Artificial Sequence
  Synthetic RNA 1
  modified_base (1)..(3): 2′-O-methyl with phosphorothioate
  modified_base (29)..(33): 2′-O-methyl
uggacagcguccauacugguguuuuagagcuag

SEQ ID NO: 12 - RNA, 39 nt, Artificial Sequence; identical to SEQ ID NO: 3
  Synthetic RNA 2
  modified_base (1)..(1): phosphate, 2′-O-methyl
  modified_base (2)..(7): 2′-O-methyl
  modified_base (36)..(39): 2′-O-methyl

SEQ ID NO: 13 - RNA, 28 nt, Artificial Sequence
  Synthetic RNA 3
  modified_base (1)..(1): phosphate, 2′-O-methyl
  modified_base (2)..(24): 2′-O-methyl
  modified_base (25)..(27): 2′-O-methyl with phosphorothioate
  modified_base (28)..(28): 2′-O-methyl
gaaaaaguggcaccgagucggugcuuuu

SEQ ID NO: 14 - DNA, 36 bp, Artificial Sequence
  Synthetic Splint 1 alt
ccttattttaacttgctatttctagctctaaaacac

SEQ ID NO: 15 - RNA, 33 nt, Artificial Sequence; identical to SEQ ID NO: 11
  Synthetic RNA 1

SEQ ID NO: 16 - RNA, 110 nt, Artificial Sequence
  Synthetic SpyCas9 sgRNA
  misc_feature (1)..(30): n, if present, is a, c, g, or u
  misc_feature (16)..(30): n may or may not be present
nnnnnnnnnnnnnnnnnnnnnnnnnnnnnnguuuuagagcuagaaauagcaaguuaaaauaaggcuaguccguuaucaacuugaaaaaguggcaccgagucggugcuuuu

SEQ ID NO: 17 - RNA, 80 nt, Artificial Sequence
  Synthetic SpyCas9 sgRNA backbone
guuuuagagcuagaaauagcaaguuaaaauaaggcuaguccguuaucaacuugaaaaaguggcaccgagucggugcuuuu

SEQ ID NO: 18 - RNA, 20 nt, Artificial Sequence
  Synthetic G6PC Spacer Sequence unmodified
uggacagcguccauacuggu

SEQ ID NO: 19 - RNA, 100 nt, Artificial Sequence
  Synthetic Unmodified G6PC sgRNA
uggacagcguccauacugguguuuuagagcuagaaauagcaaguuaaaauaaggcuaguccguuaucaacuugaaaaaguggcaccgagucggugcuuuu

SEQ ID NO: 20 - RNA, 100 nt, Artificial Sequence; identical to SEQ ID NO: 19
  Synthetic Modified G6PC sgRNA
  modified_base (1)..(3): 2′-O-methyl with phosphorothioate
  modified_base (29)..(40): 2′-O-methyl
  modified_base (69)..(96): 2′-O-methyl
  modified_base (97)..(99): 2′-O-methyl with phosphorothioate
  modified_base (100)..(100): 2′-O-methyl

SEQ ID NO: 21 - RNA, 20 nt, Artificial Sequence
  Synthetic G6PC Spacer sequence modified
  modified_base (1)..(3): 2′-O-methyl with phosphorothioate
uggacagcguccauacuggu

SEQ ID NO: 22 - DNA, 15 bp, Artificial Sequence
  Synthetic DNA splint 1 complement to RNA 1
ctagctctaaaactc

SEQ ID NO: 23 - RNA, 15 nt, Artificial Sequence
  Synthetic RNA 1 complement to DNA splint 1
guguuuuagagcuag

SEQ ID NO: 24 - DNA, 21 bp, Artificial Sequence
  Synthetic DNA splint 1 complement to RNA 2
ccttattttaacttgctattt

SEQ ID NO: 25 - RNA, 21 nt, Artificial Sequence
  Synthetic RNA 2 complement to DNA splint 1
aaauagcaaguuaaaauaagg

SEQ ID NO: 26 - DNA, 18 bp, Artificial Sequence
  Synthetic DNA splint 2 complement to RNA 2
aagttgataacggactag

SEQ ID NO: 27 - RNA, 18 nt, Artificial Sequence
  Synthetic RNA 2 complement to DNA splint 2
cuaguccguuaucaacuu

SEQ ID NO: 28 - DNA, 28 bp, Artificial Sequence
  Synthetic DNA splint 2 complement to RNA 3
aaaagcaccgactcggtgccactttttc

SEQ ID NO: 29 - RNA, 28 nt, Artificial Sequence
  Synthetic RNA 3 complement to DNA splint 2
gaaaaaguggcaccgagucggugcuuuu

SEQ ID NO: 30 - DNA, 14 bp, Artificial Sequence
  Synthetic DNA splint 1 complement to RNA 1
tctagctctaaaac

SEQ ID NO: 31 - RNA, 14 nt, Artificial Sequence
  Synthetic RNA 1 complement to DNA splint 1
guuuuagagcuaga

SEQ ID NO: 32 - DNA, 18 bp, Artificial Sequence
  Synthetic DNA splint 1 complement to RNA 2
ttattttaacttgctatt

SEQ ID NO: 33 - RNA, 18 nt, Artificial Sequence
  Synthetic RNA 2 complement to DNA splint 1
aauagcaaguuaaaauaa

SEQ ID NO: 34 - DNA, 16 bp, Artificial Sequence
  Synthetic DNA splint 2 complement to RNA 2
tgataacggactagcc

SEQ ID NO: 35 - RNA, 16 nt, Artificial Sequence
  Synthetic RNA 2 complement to DNA splint 2
ggcuaguccguuauca

SEQ ID NO: 36 - DNA, 20 bp, Artificial Sequence
  Synthetic DNA splint 2 complement to RNA 3
tcggtgccactttttcaagt

SEQ ID NO: 37 - RNA, 20 nt, Artificial Sequence
  Synthetic RNA 3 complement to DNA splint 2
acuugaaaaaguggcaccga

SEQ ID NO: 38 - RNA, 34 nt, Artificial Sequence
  Synthetic Unmodified RNA 1
uggacagcguccauacugguguuuuagagcuaga

SEQ ID NO: 39 - RNA, 34 nt, Artificial Sequence; identical to SEQ ID NO: 38
  Synthetic Modified RNA 1
  modified_base (1)..(3): 2′-O-methyl with phosphorothioate
  modified_base (29)..(34): 2′-O-methyl

SEQ ID NO: 40 - RNA, 34 nt, Artificial Sequence
  Synthetic Unmodified RNA 2
  modified_base (1)..(1): phosphate
aauagcaaguuaaaauaaggcuaguccguuauca

SEQ ID NO: 41 - RNA, 34 nt, Artificial Sequence; identical to SEQ ID NO: 40
  Synthetic Modified RNA 2
  modified_base (1)..(1): phosphate, 2′-O-methyl
  modified_base (2)..(6): 2′-O-methyl

SEQ ID NO: 42 - RNA, 32 nt, Artificial Sequence
  Synthetic Unmodified RNA 3
  modified_base (1)..(1): phosphate
acuugaaaaaguggcaccgagucggugcuuuu

SEQ ID NO: 43 - RNA, 32 nt, Artificial Sequence; identical to SEQ ID NO: 42
  Synthetic Modified RNA 3
  modified_base (1)..(1): phosphate, 2′-O-methyl
  modified_base (2)..(28): 2′-O-methyl
  modified_base (29)..(31): 2′-O-methyl with phosphorothioate
  modified_base (32)..(32): 2′-O-methyl

SEQ ID NO: 44 - DNA, 32 bp, Artificial Sequence
  Synthetic DNA Splint 1
ttattttaacttgctatttctagctctaaaac

SEQ ID NO: 45 - DNA, 36 bp, Artificial Sequence
  Synthetic DNA Splint 2
tcggtgccactttttcaagttgataacggactagcc

SEQ ID NO: 46 - DNA, 23 bp, Artificial Sequence
  Synthetic DNA splint 1 complement to RNA 1
tctagctctaaaacaccagtatg

SEQ ID NO: 47 - RNA, 23 nt, Artificial Sequence
  Synthetic RNA 1 complement to DNA splint 1
cauacugguguuuuagagcuaga

SEQ ID NO: 48 - DNA, 17 bp, Artificial Sequence
  Synthetic DNA splint 1 complement to RNA 2
tattttaacttgctatt

SEQ ID NO: 49 - RNA, 17 nt, Artificial Sequence
  Synthetic RNA 2 complement to DNA splint 1
aauagcaaguuaaaaua

SEQ ID NO: 50 - DNA, 17 bp, Artificial Sequence
  Synthetic DNA splint 2 complement to RNA 2
tgataacggactagcct

SEQ ID NO: 51 - RNA, 17 nt, Artificial Sequence
  Synthetic RNA 2 complement to DNA splint 2
aggcuaguccguuauca

SEQ ID NO: 52 - DNA, 40 bp, Artificial Sequence
  Synthetic DNA Splint 1
tattttaacttgctatttctagctctaaaacaccagtatg

SEQ ID NO: 53 - DNA, 40 bp, Artificial Sequence
  Synthetic DNA Splint 2
gactcggtgccactttttcaagttgataacggactagcct

SEQ ID NO: 54 - DNA, 23 bp, Artificial Sequence
  Synthetic DNA splint 2 complement to RNA 3
gactcggtgccactttttcaagt

SEQ ID NO: 55 - RNA, 23 nt, Artificial Sequence
  Synthetic RNA 3 complement to DNA splint 2
acuugaaaaaguggcaccgaguc

SEQ ID NO: 56 - RNA, 43 nt, Artificial Sequence
  Synthetic RNA 1
  misc_feature (1)..(30): n, if present, is a, c, g, or u
  misc_feature (16)..(30): n may or may not be present
nnnnnnnnnnnnnnnnnnnnnnnnnnnnnnguuuuagagcuag

SEQ ID NO: 57 - RNA, 44 nt, Artificial Sequence
  Synthetic RNA 1
  misc_feature (1)..(30): n, if present, is a, c, g, or u
  misc_feature (16)..(30): n may or may not be present
nnnnnnnnnnnnnnnnnnnnnnnnnnnnnnguuuuagagcuaga

SEQ ID NO: 58 - RNA, 35 nt, Artificial Sequence
  Synthetic RNA 2
  modified_base (1)..(1): Phosphate
aaauagcaaguuaaaauaaggcuaguccguuauca

SEQ ID NO: 59 - RNA, 38 nt, Artificial Sequence
  Synthetic RNA 2
aauagcaaguuaaaauaaggcuaguccguuaucaacuu

SEQ ID NO: 60 - DNA, 34 bp, Artificial Sequence
  Synthetic Splint 1
ccttattttaacttgctatttctagctctaaaac

SEQ ID NO: 61 - DNA, 31 bp, Artificial Sequence
  Synthetic Splint 1
tattttaacttgctatttctagctctaaaac

(19) United States
(12) Patent Application Publication
(10) Pub. No.: US 2022/0411841 A1
(43) Pub. Date: Dec. 29, 2022

(54) METHODS OF SYNTHESIZING RNA MOLECULES

(71) Applicants: CRISPR THERAPEUTICS AG, Zug (CH); BAYER HEALTHCARE LLC, Whippany, NJ (US)

(72) Inventors: Brian Joseph Cafferty, Cambridge, MA (US); Jon Scott Munzer, Shrewsbury, MA (US); Muthusamy Jayaraman, Cambridge, MA (US); Rajat Subhra Das, Cambridge, MA (US)

(21) Appl. No.: 17/776,822

(22) PCT Filed: Nov. 25, 2020
(86) PCT No.: PCT/US2020/062342

§ 371 Date: May 13, 2022

Related U.S. Application Data
(60) Provisional application No. 62/941,174, filed on Nov. 27, 2019.

(51) Int. Cl. C12P 19/34; C12N 15/11; C12N 9/22; C12N 9/00

(52) U.S. Cl. CPC C12P 19/34; C12N 15/111; C12N 15/11; C12N 9/22; C12N 9/93; C12N 2310/531; C12N 2310/20

(57) ABSTRACT

The present disclosure relates to methods of synthesizing moderate length RNAs by splint-mediated ligation of RNA fragments.

### CROSS REFERENCE TO RELATED APPLICATIONS

This application claims the benefit of U.S. Provisional Patent Application Ser. No. 62/941,174, filed Nov. 27, 2019. The entire contents of which is incorporated herein by reference.

### TECHNICAL FIELD

The present disclosure generally relates to the field of molecular biology and biotechnology, including the synthesis of nucleic acids and methods for synthesizing RNA molecules associated with endonucleases, also known as guide RNAs.

### BACKGROUND

The targeting of DNA using the RNA-guided, DNA-targeting principle of CRISPR (Clustered Regularly Interspaced Short Palindromic Repeats)-Cas (CRISPR associated) systems has been widely employed in the art. CRISPR-Cas systems can be divided in two classes, with class 1 systems utilizing a complex of multiple Cas proteins (such as type I, III, and IV CRISPR-Cas systems) and class 2 systems utilizing a single Cas protein (such as type II, V, and VI CRISPR-Cas systems). Type II CRISPR-Cas-based systems have been used for genome editing, and require a Cas polypeptide or variant thereof guided by a customizable guide RNA (gRNA) for programmable DNA targeting. Guide RNAs for Type II CRISPR-Cas-based systems typically range from 30-130 nucleotides in length (Chylinski et al. (2013) RNA Biology, 10(5):726-737).

Approaches for the synthesis of gRNA include, e.g., intracellular transcription of an exogenous plasmid or solid-phase synthesis using phosphoramidite chemistry. Direct chemical synthesis of gRNA allows for incorporation of chemical modifications that increase the chemical stability of the RNA, decrease its immunogenicity, and reduce potential off-target effects (i.e., cleaving genomic DNA at undesired locations). One limitation of the chemical synthesis of some sequences such as gRNAs is the length of the desired single-strand RNA, which, for gRNAs, are typically about 60 to 100 nucleotides (nts). For example, if the phosphoramidite chemistry being used has a coupling efficiency of ˜0.99^{X} (where X is the number of nucleotides), the overall synthesis process would be expected to yield approximately 30-40% full-length product (FLP) when synthesizing gRNAs with lengths on the order of 100 nucleotides. Complete isolation of the FLP from the remaining side products formed from incomplete coupling (truncation products) and deprotection is not currently achievable for RNA molecules with lengths on the order of 100 nucleotides by standard purification methods (e.g., chromatography). Because of these limitations, it is desirable to design more efficient methods of synthesizing gRNAs.

### SUMMARY

Applicants have discovered improved methods of synthesizing RNAs, particularly moderate length RNAs (mlRNAs) such as guide RNAs used in gene editing. Accordingly, the present disclosure provides methods of synthesizing mlRNAs using splint-mediated ligation of two or more RNA fragments. In some aspects, the disclosure provides methods of synthesizing mlRNAs using splint-mediated ligation of two RNA fragments or three RNA fragments. Such methods can include, e.g.: providing a first RNA fragment that includes a terminal region that includes a 5′ phosphate moiety, and a second RNA fragment that includes a terminal region that includes a 3′ hydroxyl group, where the first RNA fragment, the second RNA fragment, or both, includes at least a portion of a sequence that can, for example, bind to an RNA-guided endonuclease; providing a splint oligonucleotide that includes a first portion complementary to the first RNA fragment at the terminal region that includes a 5′ phosphate moiety and a second portion complimentary to the second RNA fragment at the terminal region that includes a 3′ hydroxyl group; hybridizing the first RNA fragment, the second RNA fragment, and the splint oligonucleotide together to form a complex; and ligating the first and second RNA fragments using a ligase at a ligation site present within the complex, thereby synthesizing an mlRNA, or portion of an mlRNA.

In some aspects, the method comprises providing: (a) a first RNA fragment comprising a terminal region comprising a 3′ hydroxyl group; (b) a second RNA fragment comprising: (i) a terminal region comprising a 5′ phosphate moiety, and (ii) a terminal region comprising a 3′ hydroxyl group; and (c) a third RNA fragment comprising a terminal region comprising a 5′ phosphate group; (d) a first splint oligonucleotide comprising (i) a first portion complementary to the terminal region comprising the 3′ hydroxyl group of the first RNA fragment; and (ii) a second portion complementary to the first terminal region comprising the 5′ phosphate moiety of the second RNA fragment; (e) a second splint oligonucleotide comprising (i) a first portion complementary to the second terminal region comprising the 3′ hydroxyl group of the second RNA fragment; and (ii) a second portion complementary to the terminal region comprising the 5′ phosphate moiety of the third RNA fragment; and (f) a ligase, wherein hybridizing the first RNA fragment, the second RNA fragment, the third RNA fragment, the first splint oligonucleotide, and the second splint oligonucleotide together forms a complex, the complex comprising a first ligation site between the 3′ hydroxyl group of the first RNA fragment and the 5′ phosphate group of the second RNA fragment, and a second ligation site between the 3′ hydroxyl group of the second RNA fragment and the 5′ phosphate group of the third RNA fragment, wherein the ligase results in a ligation of the first and second RNA fragments at the first ligation site, and of the second and third RNA fragments at the second ligation site, thereby synthesizing an mlRNA, or portion of an mlRNA. In some aspects, the first RNA fragment, the second RNA fragment, the third RNA fragment, or a combination thereof, comprise at least a portion of a sequence that, for example, binds to an RNA-guided endonuclease (e.g., Cas9). In some aspects, the first RNA fragment, the second RNA fragment, the third RNA fragment, or a combination thereof comprise a spacer sequence which targets a target sequence in a target DNA (e.g., genomic DNA molecule).

In one aspect, provided herein are methods of synthesizing a guide RNA (gRNA), the methods include: providing a first RNA fragment comprising a terminal region comprising a 5′ phosphate moiety, and a second RNA fragment comprising a terminal region comprising a 3′ hydroxyl group, where the first RNA fragment, the second RNA fragment, or both, comprises at least a portion of a sequence that can bind to an RNA-guided endonuclease; providing a splint oligonucleotide comprising a first portion complementary to the first RNA fragment at the terminal region comprising a 5′ phosphate moiety and a second portion complimentary to the second RNA fragment at the terminal region comprising a 3′ hydroxyl group; hybridizing the first RNA fragment, the second RNA fragment, and the splint oligonucleotide together to form a complex; and ligating the first and second RNA fragments using a ligase at a ligation site present between the RNA complex, thereby synthesizing a gRNA. In some embodiments, the length of the first and second RNA fragments are 10 to 90 nucleotides, each. In some embodiments, the length of the second RNA fragment is 40 nucleotides or less. In some embodiments, the 5′ phosphate moiety is 5′-phosphate or 5′-phosphorothioate. In some embodiments, the ligase is a T4 DNA ligase, T4 RNA ligase I, or T4 RNA ligase II. In some embodiments, the splint oligonucleotide is a DNA or RNA oligonucleotide. In some embodiments, the length of the splint oligonucleotide is 20 to 100 nucleotides. In some embodiments, the splint oligonucleotide is attached to a solid support. In some embodiments, the length of the gRNA is 30 to 160 nucleotides. In some embodiments, the gRNA comprises a sequence that is complementary to a sequence in a target DNA. In some embodiments, the target DNA is mammalian DNA. In some embodiments, the target DNA is human DNA. In some embodiments, the ligation site corresponds to a site in a tetraloop portion of a stem-loop structure in the synthesized gRNA. In some embodiments, the ligation site corresponds to a site in a helix portion of a stem-loop structure in the synthesized gRNA. In some embodiments, the first RNA fragment, the second RNA fragment, or both, comprises at least one secondary structure, and hybridizing the first RNA fragment, the second RNA fragment, and the splint oligonucleotide results in a lower free energy than that of the secondary structure with the lowest free energy. In some embodiments, the methods comprise ligating three or more RNA fragments. In some embodiments, providing the first and second RNA fragments comprises synthesizing the first and second RNA fragments through enzymatic synthesis or phosphoramidite chemistry. In some embodiments, the second RNA fragment is synthesized in a 5′ to 3′ or a 3′ to 5′ direction. In some embodiments, providing the first and second RNA fragments comprises purifying the first and second fragments after synthesis. In some embodiments, providing the splint oligonucleotide comprises synthesizing the splint oligonucleotide through enzymatic synthesis or phosphoramidite chemistry. In some embodiments, providing the splint oligonucleotide comprises purifying the splint oligonucleotide after synthesis. In some embodiments, purifying comprises purifying with a chromatographic method. In some embodiments, the chromatographic method is reversed-phase HPLC, ion-exchange chromatography, size exclusion chromatography, hydrophobic interaction chromatography, affinity chromatography, or polyacrylamide gel purification, or any combination thereof. In some embodiments, the first RNA fragment, the second RNA fragment, or both, comprises at least one modification in the RNA backbone. In some embodiments, the modification is selected from the group consisting of: 2′ methoxy (2′OMe), 2′ fluorine (2′fluoro), 2′-O-methoxy-ethyl (MOE), Locked Nucleic Acids (LNA), Unlocked Nucleic Acids (UNA), bridged nucleic acids, 2′deoxynucleic acids (DNA), and peptide nucleic acids (PNA). In some embodiments, the first RNA fragment, the second RNA fragment, or both, comprises at least one base modification. In some embodiments, the base modification is selected from the group consisting of: 2-aminopurine, inosine, thymine, 2,6-diaminopurine, 2-pyrimidinone, and 5-methyl cytosine. In some embodiments, the first RNA fragment, the second RNA fragment, or both, comprises at least one phosphorothioate linkage. In some embodiments, hybridizing comprises hybridizing in a solution. In some embodiments, a concentration of the splint oligonucleotide, a concentration of the first RNA fragment, and a concentration of the second RNA fragments in the solution are about equal. In some embodiments, ligating the first and second RNA fragments is carried out at 15° C.-45° C. In some embodiments, ligating the first and second RNA fragments is carried out at about 37° C. In some embodiments, ligating the first and second RNA fragments is carried out for about 0.1 to about 48 hours. In some embodiments, ligating the first and second RNA fragments further comprises using a protease or a chelating agent. In some embodiments, the chelating agent is EDTA, EGTA, or a combination of both. In some embodiments, ligating the first and second RNA fragments further comprises using one or more crowding agents. In some embodiments, the one or more crowding agents comprise polyethylene glycol (PEG), Ficoll®, ethylene glycol, dextran, or any combination thereof. In some embodiments, ligating the first and second RNA fragments proceeds to at least 10% completion. In some embodiments, ligating the first and second RNA fragments proceeds to at least 90% completion. In some embodiments, the method further comprises purifying the gRNA after synthesis. In some embodiments, purifying the gRNA comprises purifying using a chromatographic method. In some embodiments, the chromatographic method is reversed-phase HPLC, ion-exchange chromatography, size exclusion chromatography, hydrophobic interaction chromatography, affinity chromatography, or polyacrylamide gel purification, or any combination thereof. In some embodiments, the RNA-guided endonuclease is a small Cas nuclease or a small RNA-guided endonuclease. In some embodiments, the RNA-guided endonuclease is selected from the group consisting of: a Cas9, a Cas12, aCas13, and variants thereof. In some embodiments, the RNA-guided endonuclease is a Streptococcus pyogenes Cas9 (SpyCas9) or a Staphylococcus aureus (SaCas9). In some embodiments, the RNA-guided endonuclease is a variant of Cas9, and the variant of Cas9 is selected from the group consisting of: a small Cas9, a dead Cas9 (dCas9), and a Cas9 nickase.

In another aspect, the disclosure provides a method of synthesizing a gRNA, the method comprising providing (a) a first RNA fragment comprising a terminal region comprising a 3′ hydroxyl group; (b) a second RNA fragment comprising a first terminal region comprising a 5′ phosphate moiety and a second terminal region comprising a 3′ hydroxyl group; (c) a third RNA fragment comprising a terminal region comprising a 5′ phosphate moiety; (d) a first splint oligonucleotide comprising (i) a first portion complementary to the terminal region comprising the 3′ hydroxyl group of the first RNA fragment; and (ii) a second portion complementary to the first terminal region comprising the 5′ phosphate moiety of the second RNA fragment; (e) a second splint oligonucleotide comprising (i) a first portion complementary to the second terminal region comprising the 3′ hydroxyl group of the second RNA fragment; and (ii) a second portion complementary to the terminal region comprising the 5′ phosphate moiety of the third RNA fragment; and (f) a ligase, wherein hybridizing the first, second, and third RNA fragments and the first and second splint oligonucleotides results in formation of a complex having a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment; and wherein the ligase results in a ligation of the first and second RNA fragments at the first ligation site, and of the second and third RNA fragments at the second ligation site, thereby synthesizing a gRNA. In some embodiments, the gRNA comprises 5′ to 3′ the first RNA fragment linked to the second RNA fragment by a first phosphodiester bond, and the second RNA fragment linked to the third RNA fragment by a second phosphodiester bond. In some embodiments, the first phosphodiester bond is formed between the 3′ hydroxyl group of the first RNA fragment and the 5′ phosphate group of the second RNA fragment, and wherein the second phosphodiester bond is formed between the 3′ hydroxyl group of the second RNA fragment and the 5′ phosphate group of the third RNA fragment. In some embodiments, the gRNA is a single-molecule gRNA (sgRNA). In some embodiments, the sgRNA is about 30 to about 160 nucleotides in length, or about 30, 40, 50, 60, 70, 80, 90, 100, 110, 120, 130, 140, 150, or 160 nucleotides in length. In some embodiments, the first ligation site corresponds to a site in a first stem-loop structure, wherein the first stem-loop structure is formed by hybridization of a minimum CRISPR repeat sequence and a minimum tracrRNA sequence in the synthesized gRNA. In some embodiments, site in the first stem-loop structure is in a tetraloop portion or in a helix portion. In some embodiments, the second ligation site corresponds to a site in a second stem-loop structure. In some embodiments, the second-stem loop is present in a tracrRNA sequence of the gRNA. In some embodiments, the site in the second stem-loop structure is in a tetraloop portion or in a helix portion.

In some aspects, the disclosure provides a method of synthesizing a single-molecule guide RNA (sgRNA) for use with an RNA-guided endonuclease, the method comprising: providing a complex formed between a first RNA fragment, a second RNA fragment, a third RNA fragment, first splint oligonucleotide, and a second splint oligonucleotide; and a ligase, wherein (a) the first RNA fragment comprises (i) a terminal region comprising a 3′ hydroxyl group; (b) the second RNA fragment comprises (i) a first terminal region comprising a 5′ phosphate moiety, and (ii) a second terminal region comprising a 3′ hydroxyl group; (c) the third RNA fragment comprises (i) a terminal region comprising a 5′ phosphate moiety; (d) the first splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and (e) the second splint oligonucleotide comprises (i) a first portion complementary to the second terminal region comprising the 3′ hydroxyl group of the second RNA fragment, and (ii) a second portion complementary to the terminal region comprising the 5′ phosphate moiety of the third RNA fragment, wherein the complex is formed by hybridization of (a)(i) and (d)(i), (b)(i) and (d)(ii), (b)(ii) and (e)(i), and (c)(i) and (e)(ii), wherein the complex has a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment, wherein the ligase results in a ligation at the first ligation site and a ligation at the second ligation site to form a sgRNA comprising from 5′ to 3′: a spacer sequence and an invariable sequence that binds an RNA-guided endonuclease; the invariable sequence comprising a stem loop formed between a crRNA repeat sequence and a tracrRNA anti-repeat sequence, and a 3′ tracrRNA sequence comprising at least one stem-loop, thereby synthesizing the sgRNA for use with the RNA-guided endonuclease.

In any of the foregoing or related aspects, the first ligation site corresponds to a site in the stem loop formed between the crRNA repeat sequence and the tracrRNA anti-repeat sequence. In some embodiments, the first ligation site corresponds to a site in the 5′ stem of the stem loop, in the tetraloop of the stem loop, or in the 3′ stem of the stem loop. In some embodiments, the 3′ tracrRNA sequence comprises a first stem loop, a second stem loop, and a third stem loop. In some embodiments, the 3′ tracrRNA sequence consist of a first stem loop, a second stem loop, and a third stem loop. In some embodiments, the second ligation site corresponds to a site in the first stem loop, the second stem loop, or the third stem loop. In some embodiments, the second ligation site corresponds to a site in the second stem loop. In some embodiments, the site is in the 5′ stem of the second stem loop, a site in the tetraloop of the second stem loop, or a site in the 3′ stem of the second stem loop. In some embodiments, the second ligation site corresponds to a site adjacent to the 5′ base of the second stem loop (e.g., ±1 nt, ±2 nt, ±3 nt from the 5′ base of the second stem loop) or adjacent to the 3′ base of the second stem loop (e.g., ±1 nt, ±2 nt, ±3 nt from the 3′ base of the second stem loop). In some embodiments, the first RNA fragment comprises a nucleotide sequence that is 5′ the first ligation site. In some embodiments, the second RNA fragment comprises a nucleotide sequence that is between the first ligation site and the second ligation site. In some embodiments, the third RNA fragment comprises a nucleotide sequence that is 3′ to the second ligation site.

In any of the foregoing or related aspects, the terminal region of (a)(i) comprises a nucleotide sequence of about 10 to about 30 nucleotides located at the 3′end of the first RNA fragment. In some embodiments, the terminal region of (a)(i) comprises the spacer sequence of the sgRNA. In some embodiments, the terminal region of (a)(i) does not comprise the spacer sequence of the sgRNA. In some embodiments, the 5′ terminus of the spacer sequence aligns with the 5′ terminus of the first RNA fragment, wherein the terminal region of (a)(i) comprises the spacer sequence of the sgRNA. In some embodiments, the terminal region of (a)(i) extends from the 3′terminus of the first RNA fragment to include the 1, 2, 3, 4, 5, 6, 7, 8, 9, 10, 11, 12, 13, 14, 15, 16, 17, 18, 19, or 20 nt upstream the 3′terminus of the spacer sequence. In some embodiments, the terminal region of (a)(i) extends from the 3′terminus of the first RNA fragment to immediately adjacent the 3′ terminus of the spacer sequence. In some embodiments, the first portion of (d)(i) is perfectly complementary to the terminal region of (a)(i). In some embodiments, the first portion of (d)(i) has 1, 2, or 3 mismatches relative to the terminal region of (a)(i). In some embodiments, the terminal region of (b)(i) comprises a nucleotide sequence of about 10 to about 30 nucleotides located at the 5′ end of the second RNA fragment. In some embodiments, the second portion of (d)(ii) is perfectly complementary to the terminal region of (b)(i). In some embodiments, the second portion of (d)(ii) has 1, 2, or 3 mismatches relative to the terminal region of (d)(ii). In some embodiments, the terminal region of (b)(ii) comprises a nucleotide sequence of about 10 to about 30 nucleotides located at the 3′end of the second RNA fragment. In some embodiments, the first portion of (e)(i) is perfectly complementary to the terminal region of (b)(ii). In some embodiments, the first portion of (e)(i) has 1, 2, or 3 mismatches relative to the terminal region of (b)(ii). In some embodiments, the terminal region of (c)(i) comprises a nucleotide sequence of about 10 to about 40 nucleotides located at the 5′end of the third RNA fragment. In some embodiments, the second portion of (e)(ii) is perfectly complementary to the terminal region of (c)(i). In some embodiments, the second portion of (e)(ii) has 1, 2, or 3 mismatches relative to the terminal region of (c)(i).

In any of the foregoing or related aspects, the first RNA fragment, the second RNA fragment, and the third RNA fragment are each independently about 10 to about 90 nucleotides, about 10 to about 60 nucleotides, about 10 to about 50 nucleotides, about 10 to about 40 nucleotides, about 20 to about 40 nucleotides, about 30 to about 40 nucleotides in length. In some embodiments, the first splint oligonucleotide is a DNA or RNA oligonucleotide, and wherein the second splint oligonucleotide is a DNA or RNA oligonucleotide. In some embodiments, the first splint oligonucleotide and the second splint oligonucleotide are each independently about 20 to about 100 nucleotides, about 20 to about 90 nucleotides, about 20 to about 80 nucleotides, about 20 to about 70 nucleotides, about 20 to about 60 nucleotides, about 30 to about 60 nucleotides, or about 30 to about 50 nucleotides in length.

In any of the foregoing or related aspects, the gRNA or the sgRNA comprise a spacer sequence that is complementary to a sequence in a target DNA. In some embodiments, the target DNA is mammalian DNA or human DNA. In some embodiments, the RNA-guided endonuclease is a small Cas nuclease or a small RNA-guided endonuclease. In some embodiments, the RNA-guided endonuclease is selected from the group consisting of: a Cas9, a Cas12, aCas13, and variants thereof. In some embodiments, the RNA-guided endonuclease is a Streptococcus pyogenes Cas9 (SpyCas9) or a Staphylococcus aureus (SaCas9). In some embodiments, the RNA-guided endonuclease is a variant of Cas9, and the variant of Cas9 is selected from the group consisting of: a small Cas9, a dead Cas9 (dCas9), and a Cas9 nickase.

In any of the foregoing or related aspects, the RNA-guided endonuclease is SpyCas9. In some embodiments, the invariable sequence comprises the nucleotide sequence of SEQ ID NO: 17. In some embodiments, the invariable sequence comprises a nucleotide sequence having up to 1, 2, 3, 4, 5, 6, 7, 8, 9, or 10 nucleotide deletions, insertions, or substitutions relative to SEQ ID NO: 17. In some embodiments, the first RNA fragment, the second RNA fragment, and the third RNA fragment respectively are selected from the nucleotide sequences comprising: (a)(i) N₁₅₋₃₀GUUUUAGAGCUAG (SEQ ID NO: 56), wherein N₁₅₋₃₀ corresponds to the spacer sequence; (ii) SEQ ID NO: 3; and (iii) SEQ ID NO: 4; (b)(i) N₁₅₋₃₀GUUUUAGAGCUAGA (SEQ ID NO: 57), wherein N₁₅₋₃₀ corresponds to the spacer sequence; (ii) SEQ ID NO: 40; and (iii) SEQ ID NO: 42; (c)(i) N₁₅₋₃₀GUUUUAGAGCUAG (SEQ ID NO: 56), wherein N₁₅₋₃₀ corresponds to the spacer sequence; (ii) SEQ ID NO: 58; and (iii) SEQ ID NO: 42; or (d)(i) N₁₅₋₃₀GUUUUAGAGCUAGA (SEQ ID NO: 57), wherein N₁₅₋₃₀ corresponds to the spacer sequence; (ii) SEQ ID NO: 59; and (iii) SEQ ID NO: 4. In some embodiments, the spacer sequence targets a target site in a target nucleic acid molecule (e.g., genomic DNA). In some embodiments, the spacer sequence is about 10 to about 30 nucleotides in length. In some embodiments, the spacer sequence is 18, 19, 20, 21, 22, 23, 24, or 25 nucleotides in length. In some embodiments, the spacer sequence is 19 nucleotides in length. In some embodiments, the spacer sequence is 20 nucleotides in length. In some embodiments, the spacer sequence is 21 nucleotides in length. In some embodiments, the spacer sequence is 22 nucleotides in length. In some embodiments, the first splint oligonucleotide comprises the nucleotide sequence set forth in SEQ ID NO: 60; SEQ ID NO: 44; or SEQ ID NO: 61. In some embodiments, no portion of the first splint oligonucleotide is complementary to the spacer sequence. In some embodiments, the first splint oligonucleotide further comprises a 3′end having a nucleotide sequence that is complementary to the spacer sequence or to the 1, 2, 3, 4, 5, 6, 7, 8, 9, or 10 nucleotide(s) present at the 3′end of the spacer sequence.

In any of the foregoing or related aspects, the first RNA fragment, the second RNA fragment, and/or the third RNA fragment comprise at least one secondary structure, wherein the complex formed by hybridizing the first, second, and third RNA fragments and the first and second splint oligonucleotides has a lower free energy than that of the secondary structure with the lowest free energy. In some embodiments, providing the first RNA fragment, the second RNA fragment, and the third RNA fragment comprises synthesis of the RNA fragments using enzymatic synthesis or phosphoramidite chemistry. In some embodiments, providing the RNA fragments further comprises purifying the RNA fragments after synthesis. In some embodiments, synthesis of the RNA fragments using phosphoramidite chemistry comprises synthesis of the first RNA fragment, synthesis of the second RNA fragment, and synthesis of the third RNA fragment each in a 5′ to 3′ or in a 3′ to 5′ direction. In some embodiments, synthesis of the RNA fragments using phosphoramidite chemistry comprises synthesis of the first RNA fragment in a 5′ to 3′ or in a 3′ to 5′ direction and synthesis of the second RNA fragment and synthesis of the third RNA fragment each in a 3′ to 5′ direction. In some embodiments, providing the first and second splint oligonucleotides comprises synthesis of the oligonucleotides using enzymatic synthesis or phosphoramidite chemistry. In some embodiments, providing the splint oligonucleotides further comprises purifying the oligonucleotides after synthesis. In some embodiments, the first RNA fragment, the second RNA fragment, and/or the third RNA fragment, comprises at least one modification in the RNA backbone. In some embodiments, the modification is selected from the group consisting of: 2′ methoxy (2′OMe), 2′ fluorine (2′fluoro), 2′-O-methoxy-ethyl (MOE), Locked Nucleic Acids (LNA), Unlocked Nucleic Acids (UNA), bridged nucleic acids, 2′deoxynucleic acids (DNA), and peptide nucleic acids (PNA). In some embodiments, the modification is 2′-O-methylation of one or more nucleotides present in the RNA backbone. In some embodiments, the first RNA fragment, the second RNA fragment, and/or the third RNA fragment, comprises at least one base modification. In some embodiments, the base modification is selected from the group consisting of: 2-aminopurine, inosine, thymine, 2,6-diaminopurine, 2-pyrimidinone, and 5-methyl cytosine. In some embodiments, the first RNA fragment, the second RNA fragment, and/or the third RNA fragment, comprises at least one phosphorothioate linkage. In some embodiments, the hybridizing is performed in a solution. In some embodiments, the hybridizing is performed without an annealing step. In some embodiments, the hybridizing is performed with an annealing step. In some embodiments, the annealing step comprises (i) heating the solution to about 80° C. to about 95° C. for a period of time less than about 10 minutes (e.g., 1, 2, 3, 4, or 5 minutes); and (ii) cooling the solution at a rate of about 0.1° C. to about 2° C. per second (e.g., 1° C. per second) to a temperature used for the ligation (e.g., about 15° C. to about 40° C., or about 30° C., 31° C., 32° C., 33° C., 34° C., 35° C., 36° C., 37° C., 38° C., 39° C., or 40° C.). In some embodiments, a concentration of the first splint oligonucleotide, a concentration of the second splint oligonucleotide, a concentration of the first RNA fragment, a concentration of the second RNA fragment, and a concentration of the third RNA fragment in the solution are about equal. In some embodiments, the concentration is about 5 μM to about 50 μM. In some embodiments, the concentration is about 5 μM, about 10 μM, about 15 μM, about 20 μM, or about 25 μM. In some embodiments, the ligation is carried out at about 15° C. to about 45° C., or about 30° C., 31° C., 32° C., 33° C., 34° C., 35° C., 36° C., 37° C., 38° C., 39° C., or 40° C. In some embodiments, the ligation is carried out for about 0.1 to about 48 hours, or about 10, 11, 12, 13, 14, 15, 16, 17, 18, 19, or 20 hours. In some embodiments, ligating the first and second RNA fragments further comprises using a protease or a chelating agent. In some embodiments, the chelating agent is EDTA, EGTA, or a combination of both. In some embodiments, ligating the first and second RNA fragments further comprises using one or more crowding agents. In some embodiments, the one or more crowding agents comprise polyethylene glycol (PEG), Ficoll®, ethylene glycol, dextran, or any combination thereof. In some embodiments, the ligation proceeds to at least about 10%, 20%, 30%, 40%, 50%, 60%, 70%, 80%, 90%, or 100% completion. In some embodiments, the method further comprises purifying the gRNA or sgRNA after synthesis. In some embodiments, purifying the gRNA or sgRNA comprises purifying using a chromatographic method. In some embodiments, the chromatographic method is reversed-phase HPLC, ion-exchange chromatography, size exclusion chromatography, hydrophobic interaction chromatography, affinity chromatography, or polyacrylamide gel purification, or any combination thereof.

In another aspect, provided herein are methods of generating a double-molecule gRNA comprising a crRNA and a tracrRNA, the method comprising: providing a first RNA fragment comprising a terminal region comprising a 5′ phosphate moiety, and a second RNA fragment comprising a terminal region comprising a 3′ hydroxyl group, where the first RNA fragment, the second RNA fragment, or both, comprises at least a portion of a sequence that can bind to an RNA-guided endonuclease; providing a splint oligonucleotide comprising a first portion complementary to the first RNA fragment at the terminal region comprising a 5′ phosphate moiety and a second portion complimentary to the second RNA fragment at the terminal region comprising a 3′ hydroxyl group; hybridizing the first RNA fragment, the second RNA fragment, and the splint oligonucleotide together to form a complex; ligating the first and second RNA fragments using a ligase at a ligation site present between the RNA complex, thereby synthesizing a tracr RNA; providing a crRNA that comprises a sequence that is complementary to a sequence in a target DNA; and allowing the tracrRNA and crRNA to hybridize, thereby generating a double-molecule gRNA. In some embodiments, providing the crRNA comprises synthesizing the crRNA through enzymatic synthesis or phosphoramidite chemistry.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims

### BRIEF DESCRIPTION OF THE DRAWINGS

FIG. 1 is a schematic diagram showing the synthesis of a gRNA molecule by ligating two RNA fragments using a splint oligonucleotide.

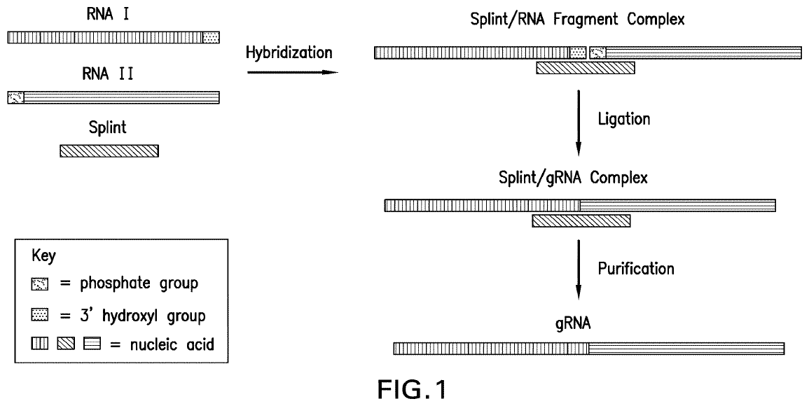

FIG. 2 is a schematic diagram showing the synthesis of a gRNA molecule by ligating three RNA fragments using two splint oligonucleotides.

FIG. 3 is a schematic diagram showing the generation of an invariable RNA construct and subsequent ligation with an additional RNA fragment, using a splint oligonucleotide, to generate a gRNA molecule.

FIG. 4 is an illustration showing locations for segmenting an exemplary gRNA molecule (SEQ ID NO: 16) into RNA fragments (RNA fragment 1, RNA fragment 2, and RNA fragment 3 having nucleotide sequences set forth in SEQ ID NOs: 56, 3, and 4 respectively).

FIG. 5 is a chromatogram showing results of HPLC analyses before and after splint-mediated ligation of RNA fragments 1-3.

FIG. 6 shows cleavage of a plasmid containing a target DNA sequence by SpCas9 and gRNA generated using splint-mediated ligation.

FIGS. 7A and 7B show examples of RNA modifications and splint mediated ligations of modified RNA fragments. FIG. 7A shows structures of unmodified ribonucleotide, 2′-O-methyl and phosphorothioate modified ribonucleotide. FIG. 7B is a chromatogram showing results of HPLC analyses of splint-mediated ligation products.

FIG. 8 is a chromatogram showing results of HPLC analyses of splint-mediated ligation products before and after purification.

FIGS. 9A-9B provide schematics for the splint-mediated ligation between three modified RNA fragments (RNA 1, RNA 2, and RNA 3) and two DNA splint oligonucleotides (DNA splint 1 and DNA splint 2) to generate a modified single-molecule gRNA (sgRNA) for use with S. pyogenes Cas9 (SpyCas9) to target the human G6PC gene. The sequences for RNA 1 (SEQ ID NO: 11), RNA 2 (SEQ ID NO: 12), and RNA 3 (SEQ ID NO: 13) and ligation sites between the RNA fragments are shown in FIG. 9B.

FIG. 10 is a chromatogram showing results of HPLC analyses of the splint-mediated ligation reaction represented in FIGS. 9A-9B for mixtures analyzed (i) before addition of ligase (“before ligation”); (ii) after addition of ligase with or without added magnesium salt (“after ligation with Mg²⁺” and “after ligation without Mg²⁺” respectively), or (iii) after addition of ligase performed without prior annealing of the RNA fragments and DNA splints (“after ligation without annealing”). Full-length sgRNA (SEQ ID NO: 20) corresponds to the peak having retention time of 30.58 min.

FIG. 11 provides an alignment of three RNA fragments and two DNA splints that form an RNA/DNA complex for a splint-mediated ligation to synthesize a sgRNA. The 5′ to 3′ nucleotide sequence is the DNA version of the sgRNA final product (SEQ ID NO: 19). The sgRNA is synthesized by hybridization of RNA 1, RNA 2 and RNA 3 with DNA splint 1 and DNA splint 2, and subsequent ligation at a ligation site between the 3′terminus of RNA 1 and the 5′terminus of RNA 2, and a ligation site between the 3′terminus of RNA 2 and the 5′terminus of RNA 3. The nucleotide sequence of DNA splint 1 and DNA splint 2 is shown in the 3′ to 5′ direction, and corresponds to SEQ ID NOs: 52 and 53 respectively.

### DETAILED DESCRIPTION

The present disclosure provides methods for synthesizing RNAs, particularly moderate length RNAs (mlRNAs), such as guide RNAs (gRNAs) by ligating RNA fragments using one or more splint oligonucleotides and a ligase. In some instances, one or more of the RNA fragments include at least a portion of a sequence that can bind to an RNA-guided endonuclease. In some embodiments, one or more of the RNA fragments comprise a spacer sequence for targeting a target sequence in a target DNA (e.g., genomic DNA molecule).

Current approaches for the synthesis of mlRNAs include, e.g., intracellular transcription of an exogenous plasmid or solid-phase synthesis using phosphoramidite chemistry. One limitation for the chemical synthesis of mlRNAs is the length of the resulting single-strand RNA. For example, if the phosphoramidite chemistry being used has a coupling efficiency of ˜0.99^{X} (where X is the number of nucleotides), the overall synthesis process would be expected to yield approximately 30-40% full-length product (FLP) when synthesizing RNAs with lengths on the order of 100 nucleotides. Complete isolation of the FLP from the remaining side products formed from incomplete coupling (truncation products) and deprotection is not currently achievable for RNA molecules with lengths on the order of 100 nucleotides using standard purification methods (e.g. chromatography). The present disclosure provides more efficient methods of synthesizing mlRNAs which improves the yield of full-length products, and decreases the number of truncation products produced. Moreover, it is demonstrated herein the methods of the disclosure provide for synthesis of both unmodified mlRNAs (e.g., a gRNA or sgRNA) and mlRNAs comprising one or more chemical modifications, such as, a backbone modification (e.g., a phosphorothioate linkage) and/or a nucleoside modification (e.g., 2′-O-methylation).

The present disclosure is also based, at least in part, on the discovery that single-molecule gRNAs (sgRNAs) for use with an RNA-guided endonuclease (e.g., Cas9) are effectively synthesized using a splint-mediated ligation approach described herein, such as, the ligation of two or three RNA fragments using one or more splint oligonucleotides, and a ligase. In some aspects, the ligation comprises two RNA fragments, one splint oligonucleotide, and a ligase, wherein the two RNA fragments hybridize the splint oligonucleotide to form a complex comprising a ligation site, and wherein the ligase results in ligation at the ligation site, thereby forming the sgRNA for use with an RNA-guided endonuclease. In some aspects, the sgRNA comprises a nucleotide sequence that is 5′ the ligation site and a nucleotide sequence that is 3′ the ligations site, wherein a first RNA fragment corresponds to the nucleotide sequence that is 5′ the ligation site, and second RNA fragment corresponds to the nucleotide sequence that is 3′ the ligation site, wherein ligation at the ligation site enables joining of the first and second RNA fragments to form the nucleotide sequence of the sgRNA.

In some aspects, the ligation comprises three RNA fragments, two splint oligonucleotides, and a ligase, wherein the three RNA fragments hybridize the two splint oligonucleotides to form a complex comprising a first and second ligation site, and wherein the ligase results in ligation at the first and second ligation site, thereby forming the sgRNA for use with an RNA-guided endonuclease. In some aspects, the sgRNA comprises a nucleotide sequence that is 5′ the first ligation site, a nucleotide sequence that is 3′ the first ligation site and 5′ the second ligation site, and a nucleotide sequence that is 3′ the second ligation site, wherein a first RNA fragment corresponds to the nucleotide sequence that is 5′ the first ligation site, a second RNA fragment corresponds to the nucleotide sequence that is 3′ the first ligation site and 5′ the second ligation site, and a third RNA fragment corresponds to the nucleotide sequence 3′ the second ligation site, wherein ligation at the first and second ligation sites enables joining of the first, second, and third RNA fragments to form the nucleotide sequence of the sgRNA.

In some aspects, the nucleotide sequence of the sgRNA comprises 5′ to 3′: a spacer sequence for targeting a target site in a nucleic acid molecule (e.g., genomic DNA molecule) and an invariable sequence that binds the RNA-guided endonuclease, the invariable sequence comprising 5′ to 3′: a stem loop formed between a CRISPR repeat sequence and a tracrRNA anti-repeat sequence, and a tracrRNA comprising at least one stem loop. In some aspects, the splint-mediated ligation approach provides at least one RNA fragment, or a combination of RNA fragments, comprising the spacer sequence; and at least one RNA fragment, or a combination of RNA fragments, comprising an invariable sequence.

In some aspects, the splint-mediated ligation approach comprises placement of a ligation site in a sgRNA, the ligation site within or proximal a stem loop in the invariable sequence of the sgRNA (e.g., within or proximal a stem loop formed between the CRISPR repeat sequence and the tracrRNA anti-repeat sequence; e.g., within or proximal a stem loop of the tracrRNA). As described herein, placement of the ligation site in a stem loop prevents formation of secondary structure in the RNA fragments (i.e., the RNA fragments joined at the ligation site) that would prevent or disfavor hybridization of the RNA fragments with a splint oligonucleotide. For example, disruption of a stem loop by a ligation site provides RNA fragments (i.e., RNA fragments joined at the ligation site) that have (i) minimal secondary structure; and/or (ii) secondary structure with free energy higher that is higher than the free energy resulting from hybridization of the RNA fragments and the splint oligonucleotide.

In some aspects, the splint-mediated ligation approach comprises placement of a first and second ligation site in the sgRNA, the first and second ligation site each within or proximal a stem loop in the invariable sequence of the sgRNA (e.g., a stem loop formed between the CRISPR repeat sequence and the tracrRNA anti-repeat sequence; e.g., a stem loop of the tracrRNA), such that placement of the first ligation site disrupts stem loop formation in an RNA fragment comprising a nucleotide sequence 5′ the first ligation site and an RNA fragment comprising a nucleotide sequence 3′ the first ligation site; and placement of the second ligation site disrupts stem loop formation in the RNA fragment comprising a nucleotide sequence 5′ the second ligation site and an RNA fragment comprising a nucleotide sequence 3′ the second ligation site, such that, formation of secondary structure in each or all RNA fragments used in the splint-mediated ligation reaction is disfavored relative to hybridization with a splint oligonucleotide.

In the following detailed description, reference is made to the accompanying drawings. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art.

By “moderate length RNA (mlRNAs)” is meant an RNA molecule with a length of about 30 to about 160 nucleotides (e.g., about 30 to about 150, about 30 to about 140, about 30 to about 130, about 30 to about 120, about 30 to about 110, about 30 to about 100, about 30 to about 90, about 30 to about 80, about 30 to about 70, about 30 to about 60, about 30 to about 50, about 30 to about 40, about 40 to about 160, about 40 to about 150, about 40 to about 140, about 40 to about 130, about 40 to about 120, about 40 to about 110, about 40 to about 100, about 40 to about 90, about 40 to about 80, about 40 to about 70, about 40 to about 60, about 40 to about 50, about 50 to about 160, about 50 to about 150, about 50 to about 140, about 50 to about 130, about 50 to about 120, about 50 to about 110, about 50 to about 100, about 50 to about 90, about 50 to about 80, about 50 to about 70, about 50 to about 60, about 60 to about 160, about 60 to about 150, about 60 to about 140, about 60 to about 130, about 60 to about 120, about 60 to about 110, about 60 to about 100, about 60 to about 90, about 60 to about 80, about 60 to about 70, about 70 to about 160, about 70 to about 150, about 70 to about 140, about 70 to about 130, about 70 to about 120, about 70 to about 110, about 70 to about 100, about 70 to about 90, about 70 to about 80, about 80 to about 160, about 80 to about 150, about 80 to about 140, about 80 to about 130, about 80 to about 120, about 80 to about 110, about 80 to about 100, about 80 to about 90, about 90 to about 160, about 90 to about 150, about 90 to about 140, about 90 to about 130, about 90 to about 120, about 90 to about 110, about 90 to about 100, about 100 to about 160, about 100 to about 150, about 100 to about 140, about 100 to about 130, about 100 to about 120, about 100 to about 110, about 110 to about 160, about 110 to about 150, about 110 to about 140, about 110 to about 130, about 110 to about 120, about 120 to about 160, about 120 to about 150, about 120 to about 140, about 120 to about 130, about 130 to about 160, about 130 to about 150, about 130 to about 140, about 140 to about 160, about 140 to about 150, or about 150 to about 160 nucleotides).

By “RNA-guided endonuclease” is meant a polypeptide capable of binding an RNA (e.g., a gRNA) to form a complex targeted to a specific DNA sequence (e.g., in a target DNA). An exemplary RNA-guided endonuclease is a Cas polypeptide (e.g., a Cas endonuclease, such as a Cas9 endonuclease). Thus, in some embodiments, an RNA-guided endonuclease as described herein is targeted to a specific DNA sequence in a target DNA by an RNA molecule to which it is bound. The RNA molecule includes a sequence that is complementary to and capable of hybridizing with a target sequence within the target DNA, thus allowing for targeting of the bound polypeptide to a specific location within the target DNA.

A “guide RNA” or “gRNA” as used herein is a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

As used herein, a “secondary structure” of a nucleic acid molecule (e.g., an RNA fragment, or a gRNA) refers to the base pairing interactions within the nucleic acid molecule.

A “target DNA” as used herein is a DNA that includes a “target site” or “target sequence.” The term “target sequence” is used herein to refer to a nucleic acid sequence present in a target DNA to which a DNA-targeting sequence or segment (also referred to herein as a “spacer”) of a gRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a gRNA and the target sequence can, for example, be based on Watson-Crick base pairing rules, which enables programmability in the DNA-targeting sequence or segment. The DNA-targeting sequence or segment of a gRNA can be designed, for instance, to hybridize with any target sequence.

The term “Cas endonuclease” or “Cas nuclease” as used herein includes, but is not limited to, for example, an RNA-guided DNA endonuclease associated with the CRISPR adaptive immunity system.

Unless otherwise indicated “nuclease” and “endonuclease” are used interchangeably herein to mean an enzyme which possesses endonucleolytic catalytic activity for polynucleotide cleavage.

“Cleavage” as used herein means the breakage of the covalent backbone of a DNA molecule. Both single-stranded cleavage and double-stranded cleavage are possible, and double-stranded cleavage can occur as a result of two distinct single-stranded cleavage events.

The term “domain” is used herein to describe segments of a protein or nucleic acid. Unless otherwise indicated, a domain need not have any specific functional property.

A “splint oligonucleotide” is an oligonucleotide that, when hybridized to other polynucleotides (e.g., RNA fragments), acts as a “splint” to position the ends of the polynucleotides next to one another so that they can be ligated together. The splint oligonucleotide can be any oligomer that can hybridize with the polynucleotides through Watson-Crick base-pairing interactions. The splint oligonucleotide can be DNA, RNA, non-natural, or artificial nucleic acids (e.g., peptide nucleic acids). The splint oligonucleotide can include a nucleotide sequence that is partially complimentary to nucleotide sequences from two or more different oligonucleotides. In general, an RNA ligase, a DNA ligase, or another variety of ligase can be used to ligate two nucleotide sequences together.

The “spacer” or “variable region” of a gRNA includes a nucleotide sequence that is complementary to a specific sequence within a target DNA (the complementary strand of the target DNA). In some aspects, the spacer confers target specificity to the gRNA combined with an RNA-guided endonuclease, enabling the RNA-guided endonuclease to cleave at the target site targeted by the spacer in the target DNA. As used herein, the term “spacer” is used interchangeably with the term “spacer sequence.”

The term “invariable region” of a gRNA refers to the nucleotide sequence of the gRNA that associates with the RNA-guided endonuclease. In some aspects, the gRNA comprises a crRNA and a transactivating crRNA (tracrRNA), wherein the crRNA and tracrRNA hybridize to each other to form a duplex. In some aspects, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some aspects, the invariable region of the gRNA refers to the portion of the crRNA that is the minimum CRISPR repeat sequence and the tracrRNA.

The terms “polynucleotide” and “nucleic acid” are used interchangeably herein and refer to a polymeric form of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases.

“Binding” as used herein refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. Kd is dependent on environmental conditions, e.g., pH and temperature, as is known by those in the art. “Affinity” refers to the strength of binding, and increased binding affinity is correlated with a lower Kd.

The terms “hybridizing” or “hybridize” refer to the pairing of substantially complementary or complementary nucleic acid sequences within two different molecules. Pairing can be achieved by any process in which a nucleic acid sequence joins with a substantially or fully complementary sequence through base pairing to form a hybridization complex. In some embodiments, “hybridizing” or “hybridize” comprises denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. For example, a splint oligonucleotide sequence will generally be not more than about 50% identical to one of the two polynucleotides (e.g., RNA fragments) to which it is designed to be complementary. However, the complementary portion of each sequence can be referred to herein as a ‘segment’, and the segments are substantially complementary if they have 80% or greater identity.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### I. Splint Oligonucleotides

The present disclosure provides methods of synthesizing RNAs, particularly mlRNAs such as gRNAs using splint-mediated ligation of two or more RNA fragments. In some embodiments, the method comprises use of a splint-mediated ligation of two, three, or more (e.g., four, five, six, seven, or eight) RNA fragments. Integral to these methods are splint oligonucleotides. These are hybridized with a first RNA fragment and a second RNA fragment to form a complex, which facilitates ligation of the first and second RNA fragments at a ligation site present between the RNA fragments. In some embodiments, the method comprises use of a splint-mediated ligation of two RNA fragments using one splint oligonucleotide. In some embodiments, the method comprises use of a splint-mediated ligation of three RNA fragments using two splint oligonucleotides. In some embodiments, the method comprises use of a splint-mediated ligation of more than three RNA fragments (e.g., four, five, six, seven, or eight RNA fragments) using an appropriate number of splint oligonucleotides needed to ligate the RNA fragments.

A splint oligonucleotide includes, for example, a first portion complementary to the first RNA fragment at the terminal region that includes a 5′ phosphate moiety. It further includes a second portion complimentary to the second RNA fragment at the terminal region comprising a 3′ hydroxyl group. The splint oligonucleotide can hybridize with the first RNA fragment and the second RNA fragment to form a complex. In the complex, the RNA fragments are positioned favorably for ligation at a ligation site present between the RNA fragments.

The splint oligonucleotide may include a sequence complementary to at least 1, 2, 3, 4, 5, 6, 7, 8, 9, 10, 11, 12, 13, 14, or 15 nucleotides, consecutively or non-consecutively, in the first RNA fragment at the terminal region that includes a 5′ phosphate moiety. The splint oligonucleotide may include a sequence complementary to between 1 and 20 nucleotides, between 21 and 40 nucleotides, between 41 and 60 nucleotides, or between 61 and 80 nucleotides, of the first RNA fragment, where the nucleotides can be consecutive or non-consecutive. The splint oligonucleotide may include a sequence complementary to at least 1, 2, 3, 4, 5, 6, 7, 8, 9, 10, 11, 12, 13, 14, or 15 nucleotides, consecutively or non-consecutively, in the second RNA fragment at the terminal region comprising a 3′ hydroxyl group. The splint oligonucleotide may include a sequence complementary to between 1 and 20 nucleotides, between 21 and 40 nucleotides, between 41 and 60 nucleotides, or between 61 and 80 nucleotides of the second RNA fragment, where the nucleotides can be consecutive or non-consecutive.

The length of the sequence in the splint oligonucleotide complementary to the first RNA fragment and the length of the sequence complementary to the second RNA fragment can be the same or can be different.

A splint oligonucleotide can be designed to preferentially promote complex formation between the RNA fragments and the splint oligonucleotide over intramolecular structures (e.g., secondary structures) present in the RNA fragments and/or the splint oligonucleotide. Minimum free energy prediction algorithms can be used for designing suitable splint oligonucleotides provided by the methods of the present disclosure. In theory, the lower the free energy, the more likely the complex between the RNA fragments and the splint oligonucleotide will form. The minimum free energy structure of a sequence is the secondary structure that is calculated to have the lowest value of free energy (and thus most likely to form in theory). By way of example, minimum free energy prediction algorithms can be used to calculate the free energy of the secondary structure(s) of an RNA fragment, which is represented by ΔGᵢₙₜᵣₐ, and the free energy of the intermolecular hybridization between the RNA fragment and the splint oligonucleotide, which is represented by ΔGᵢₙₜₑᵣ. In some instances, the Nearest-Neighbor approximations are used. The melting temperature (Tm) of the secondary structure(s) of an RNA fragment is represented by Tₘ₋ᵢₙₜᵣₐ. The melting temperature of the RNA fragment and splint oligonucleotide hybrid is represented by Tₘ₋ᵢₙₜₑᵣ. The length of the splint oligonucleotide can be designed to ensure that ΔGᵢₙₜᵣₐ is greater than ΔGᵢₙₜₑᵣ, and/or Tₘ₋ᵢₙₜₑᵣ is greater than Tₘ₋ᵢₙₜᵣₐ. An exemplary free energy prediction algorithm can be accessed from available from URL:://unafold.rna.albany.edu/?q=mfold.

In instances where the first RNA fragment, the second RNA fragment, or both, comprises at least one secondary structure, hybridizing the first RNA fragment, the second RNA fragment, and the splint oligonucleotide results in a lower free energy than the free energy associated with one or more of the at least one secondary structure. Hybridizing the first RNA fragment, the second RNA fragment, and the splint oligonucleotide can also result in, for example, a lower free energy than that of the secondary structure with the lowest free energy (or the minimum free energy) of the first RNA fragment, the second RNA fragment, or both.

One or more splint oligonucleotides may be used to hybridize with the RNA fragments to mediate ligation of the RNA fragments. The number of splint oligonucleotide(s) used for mediating ligation can be fewer than the number of RNA fragments to be ligated. For example, the number of the splint oligonucleotide(s) used for mediating ligation can be one fewer than the number of RNA fragments to be ligated, i.e., if the number of RNA fragments to be ligated is n, the number of the splint oligonucleotide(s) can be n−1.

The length of the splint oligonucleotide can be 20 to 100 nucleotides (e.g., 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, or 90 to 95 nucleotides).

A splint oligonucleotide can be attached to a support. The splint oligonucleotide can be attached to the support using a variety of techniques. For example, the splint oligonucleotide can be directly attached to a support, or immobilized to the support by chemical immobilization. For example, a chemical immobilization can take place between functional groups on the support and corresponding functional elements in the splint oligonucleotide. Such corresponding functional elements in the splint oligonucleotide can either be an inherent chemical group of the splint oligonucleotide, e.g. a hydroxyl group or be additionally introduced. An example of such a functional group is an amine group. Typically, the splint oligonucleotide to be immobilized includes a functional amine group or is chemically modified to include a functional amine group. Means and methods for such a chemical modification are known in the art.

The localization of the functional group within the splint oligonucleotide to be immobilized can be used to control and shape the binding behavior and/or orientation of the splint oligonucleotide, e.g., the functional group can be placed at the 5′ or 3′ end of the splint oligonucleotide or within the sequence of the splint oligonucleotide. A typical support for a splint oligonucleotide to be immobilized includes moieties which are capable of binding to such splint oligonucleotide, e.g., to amine-functionalized nucleic acids. Non-limiting examples of such supports include: carboxy, aldehyde, and epoxy supports.

Supports on which a splint oligonucleotide can be immobilized can be chemically activated, e.g. by the activation of functional groups, available on the support. The term “activated substrate” relates to a material in which interacting or reactive chemical functional groups were established or enabled by chemical modification procedures. For example, a support including carboxyl groups can be activated before use. Furthermore, certain supports contain functional groups that can react with specific moieties already present in the splint oligonucleotide.

A covalent linkage used to couple a splint oligonucleotide to a support can be viewed as both a direct and indirect linkage, in that although the splint oligonucleotide is attached by a “direct” covalent bond, there can be a chemical moiety or linker separating the “first” nucleotide of the splint oligonucleotide from the support, i.e., an indirect linkage. In some instances, splint oligonucleotides that are immobilized to the support by a covalent bond and/or chemical linker are generally seen to be immobilized or attached directly to the support. A splint oligonucleotide may not bind directly to the support, but interacts indirectly, for example by binding to a molecule which itself binds directly or indirectly to the support. The splint oligonucleotide can also be indirectly attached to a support (e.g., via a solution including a polymer).

In instances where the splint oligonucleotide is immobilized on the support indirectly, e.g., via hybridization to a surface oligonucleotide capable of binding the splint oligonucleotide, the splint oligonucleotide can further include an upstream sequence (5′ to the sequence that hybridizes to the two or more RNA fragments as described herein) that is capable of hybridizing to the 5′ end of the surface oligonucleotide.

The splint oligonucleotide can be attached to the support via its 5′ end or its 3′ end. The splint oligonucleotide attached to the support can be in situ synthesized on the support.

### II. Methods of Synthesizing RNAs

The presently disclosed methods of synthesizing mlRNAs generally include providing a first RNA fragment, a second RNA fragment, and a splint oligonucleotide. The first RNA fragment, the second RNA fragment, and the splint oligonucleotide are hybridized together to form a complex. Forming such a complex positions the first and second RNA fragments in close proximity to facilitate ligation. A ligase is then used to ligate the first and second RNA fragments across the ligation site, thereby synthesizing an mlRNA.

In some embodiments, the methods comprise providing a first RNA fragment, a second RNA fragment, a third RNA fragment, a first splint oligonucleotide, and a second oligonucleotide. The first RNA fragment, the second RNA fragment, and the first splint oligonucleotide are hybridized together; and the second RNA fragment, the third RNA fragment, and the second splint oligonucleotides are hybridized together, thereby forming a complex comprising the first, second, and third RNA fragments, and the first and second splint oligonucleotides. Formation of the complex positions (i) a 3′ hydroxyl group of the first RNA fragment and a 5′ phosphate moiety of the second RNA fragment in close proximity to provide a first ligation site; and (ii) a 3′ hydroxyl group of the second RNA fragment and a 5′ phosphate moiety of the third RNA fragment in close proximity to provide a second ligation site. The method further provides a ligase to ligate the first and second RNA fragments at the first ligation site, and to ligate the second and third RNA fragments at the second ligation site, thereby synthesizing an mlRNA.

Hybridizing the first RNA fragment, the second RNA fragment, and the splint oligonucleotide can be performed in a solution. In some embodiments, the hybridizing further comprises a third RNA fragment and a second splint oligonucleotide, which is performed in the solution. When hybridizing in solution, the concentration of the first RNA fragment can be, e.g., about equal to a concentration of the second RNA fragment. In some embodiments, wherein the hybridization further comprises a third RNA fragment, the concentration of the first RNA fragment and the second RNA fragment are each about equal to the concentration of the third RNA fragment. Depending upon the methods, fragments, and splint oligonucleotide(s) employed, the concentration of the splint oligonucleotide in the solution may be about equal to, more than, or less than, a concentration of the first RNA fragment in the solution, or a concentration of the second RNA fragment in the solution. For example, the concentration of the splint oligonucleotide, the first RNA fragment, and the second RNA fragment can be about equal. In some embodiments, the method comprises a first, second, and third RNA fragment, and a first and second splint oligonucleotide, wherein the concentration of the first splint oligonucleotide, the concentration of the second splint oligonucleotide, the concentration of the first RNA fragment, the concentration of the second RNA fragment, and the concentration of the third RNA fragment in the solution are each about equal.

In some instances, for hybridizing, the RNA fragments and/or the splint oligonucleotide are denatured, i.e., the intramolecular structures of the RNA fragments and/or the splint oligonucleotide are disrupted to allow for annealing between the RNA fragments and the splint oligonucleotide. Denaturing may be achieved, for example, by heating a solution containing the RNA fragments and the splint oligonucleotide to at least about 37° C. (e.g., at least about 37° C., 38° C., 39° C., 40° C., 42° C., 44° C., 46° C., 48° C., 50° C., 52° C., 54° C., 56° C., 58° C., 60° C., 65° C., 70° C., 75° C., 80° C., 85° C., 90° C., 95° C., or at least about 100° C.). In some instances, hybridizing comprises heating the solution to a temperature of about 80° C. to about 100° C., e.g., about 82° C. to about 98° C., about 84° C. to about 96° C., about 86° C. to about 94° C., or about 88° C. to about 92° C. (e.g., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., or about 99° C.). In other instances, hybridizing does not include heating the solution.

In some instances, hybridizing includes cooling the solution to a temperature of about 20° C. to about 45° C., e.g., about 22° C. to about 43° C., about 25° C. to about 40° C., or about 27° C. to about 38° C. (e.g., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., or about 44° C.) after heating. For example, in some instances, hybridizing includes cooling the solution to about 37° C. after heating. Hybridizing can include cooling the solution to a temperature at which a ligase used in the presently described methods retains ligase activity sufficient to ligate the first and second RNA fragments, and/or to a temperature below the melting temperature of the complex formed by the RNA fragments and the splint oligonucleotide upon hybridization. In instances where hybridizing does not comprise heating the solution, hybridizing can be carried out at a temperature that is lower than the melting temperature of the complex formed by the RNA fragments and the splint oligonucleotide upon hybridization. Depending on the specific method being performed, cooling the solution after heating can include reducing the temperature of the solution at a constant rate or at an uncontrolled rate.

The methods described in the present disclosure include ligating the first and second RNA fragments using a ligase at a ligation site. Ligating can include ligating the 5′ phosphate group at the terminal region of the first RNA fragment with the 3′ hydroxyl group at the terminal region of the second RNA fragment. Catalyzed by the ligase, the 5′ phosphate group and the 3′ hydroxyl group can react to form a phosphodiester bond. A ligation site can be the site at which the phosphodiester bond between the 5′ phosphate group and the 3′ hydroxyl group is formed.

In some embodiments, the methods comprise ligating a first RNA fragment and a second RNA fragment using a ligase at a first ligation site, and ligating the second RNA fragment and a third RNA fragment using a ligase at a second ligation site. In some embodiments, the ligating comprises ligation of a 3′ hydroxyl group at the terminus of the first RNA fragment with a 5′ phosphate at the terminus of the second RNA fragment; and ligation of a 3′ hydroxyl group at the terminus of the second RNA fragment and a 5′ phosphate at the terminus of the third RNA fragment, each ligation resulting in formation of a phosphodiester bond.

In general, ligating the first and second RNA fragments can be carried out at a temperature of about 15° C. to about 45° C., e.g., about 17° C. to about 43° C., about 20° C. to about 40° C., or about 22° C. to about 38° C. (e.g., about 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or about 45° C.). For example, ligating the first and second RNA fragments can be carried out at 37° C. Ligating the first and second RNA fragments can be carried out for various time periods depending on the method being performed, e.g., for about 0.1 to about 48 hours, e.g., about 0.3 to about 45 hours, about 0.5 to about 40 hours, about 0.7 to about 35 hours, about 1 to about 30 hours, about 1.5 to about 25 hours (e.g., about 1, 2, 4, 6, 8, 10, 12, 14, 16, 18, 20, 25, 30, 35, 40, or about 45 hours). In some embodiments, the temperature and/or reaction time used for the splint-mediated ligation reaction is independent of the number of RNA fragments used in the reaction, for example, a reaction temperature and/or reaction time suitable for a splint-mediated ligation reaction comprising two RNA fragments is suitable for a splint-mediated ligation reaction comprising three or more RNA fragments.

In some instances, it is useful to quench the ligation reaction following synthesis of the gRNA. For example, a ligation reaction can be quenched using a protease or a chelating agent. Non-limiting examples of proteases include proteinase K. Non-limiting examples of chelating agents include EDTA and EGTA, or a combination of both.

In some instances, ligating the first and second RNA fragments further comprises using one or more crowding agents. Non-limiting examples of crowding agents include: polyethylene glycol (PEG), Ficoll®, ethylene glycol, and dextran, or any combination thereof. In some embodiments, use of one or more crowding agents is suitable in a splint-mediated ligation reaction comprising two, three, or more RNA fragments.

A variety of ligases may be used in the presently described methods. For example, the ligase can be a T4 DNA ligase, T4 RNA ligase I, T4 RNA ligase II, RtcB ligase, T3 DNA ligase, T7 DNA ligase, Taq DNA ligase, PBCV-1 DNA ligase, thermostable DNA ligase (e.g., 5′AppDNA/RNA ligase), or an ATP dependent DNA ligase. Combinations of any two or more such ligases may be used in some instances.

A T4 RNA ligase II is particularly useful in the presently described methods. In some instances, the T4 RNA ligase II can be modified. For example, the T4 RNA ligase II may be truncated and/or comprise a mutation. For example, the T4 RNA ligase II may comprise a K227Q mutation and/or a R55K mutation. In some instances, the T4 RNA ligase II can be both truncated and have a K227Q and/or a R55K mutation. Also useful in the presently described methods is a PBCV-1 DNA ligase (i.e., Chlorella virus DNA Ligase; SplintR® ligase). In some instances, the ligase can be a DNA ligase (e.g., a 9° N® DNA ligase).

In some methods described herein, three or more (e.g., three, four or five) RNA fragments can be ligated to synthesize an mlRNA. Ligation of the three or more RNA fragments can be carried out in the same step, or in separate steps (such as in a step-wise fashion).

Methods described herein can further include purifying the ml RNA after synthesis. The purification can resolve the full-length RNA product from unreacted RNA fragments and/or splint oligonucleotides. Purification can include, for example, enzymatically degrading the unreacted RNA fragments, e.g., using an exonuclease, such as one specific for 5′-monophosphate-containing RNA. An exemplary exonuclease is XRN-1.

Purification of the full-length RNA product from the unreacted RNA fragments and/or splint oligonucleotides can also be carried out using ultra-filtration or with chromatographic methods. Non-limiting examples of chromatographic methods include: reversed-phase HPLC, ion-exchange chromatography (e.g., strong anion exchange HPLC or weak anion exchange HPLC), size exclusion chromatography, hydrophobic interaction chromatography, affinity chromatography, liquid chromatography-mass spectrometry (LCMS), capillary electrophoresis (CE), capillary gel electrophoresis (CGE), and polyacrylamide gel purification.

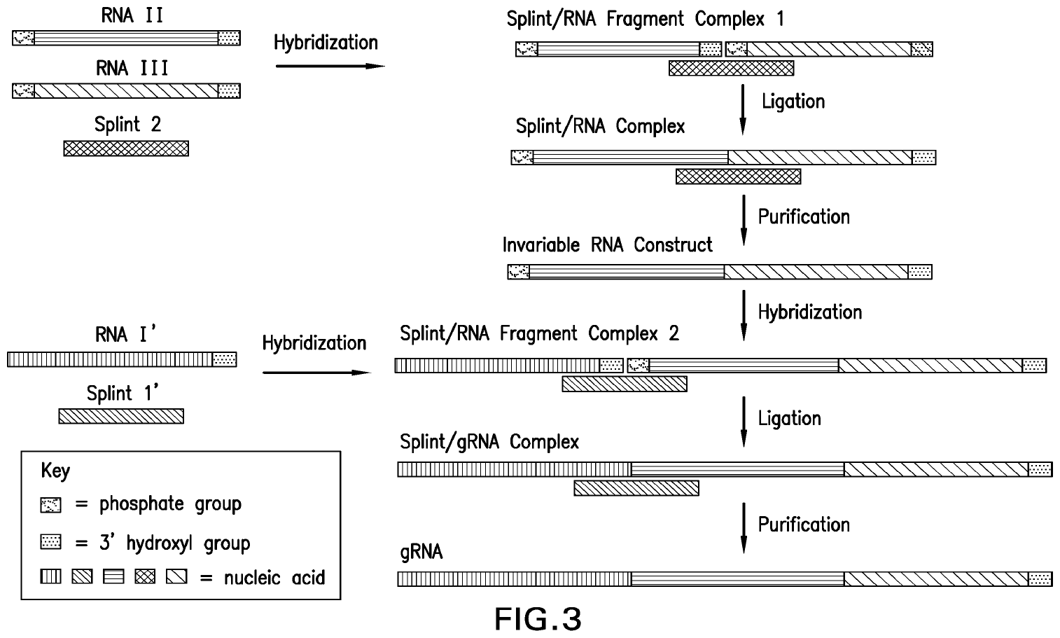

Also provided herein are methods of synthesizing an RNA molecule that includes all or a portion of a tracrRNA sequence, using any of the methods described herein. The RNA molecule can optionally be purified and used for generating a full-length gRNA (e.g., sgRNA). To generate a full-length sgRNA, an additional RNA fragment that includes a spacer and a minimum CRISPR repeat sequence (e.g., a crRNA) is ligated with the previously synthesized RNA molecule, using a splint oligonucleotide. These methods can, for example, produce full-length gRNAs specific for any target DNA sequence by ligating an RNA fragment containing the corresponding spacer sequence onto the previously synthesized RNA molecule comprising all or a portion of the tracrRNA sequence. FIG. 3 is a representative schematic showing a three-fragment system where two of the RNA fragments (fragments II and III) are ligated first, using a splint oligonucleotide, to form an invariable RNA construct that includes a portion of a tracrRNA sequence. This RNA product can, for example, be purified and stored for later use. An RNA fragment I′ that includes a sequence complementary to a specific target DNA can then be combined with the previously synthesized RNA construct to form a full-length gRNA.

### III. RNA Fragments

A method of synthesizing mlRNAs as described in the present disclosure includes providing a first RNA fragment including a terminal region that includes a 5′ phosphate moiety, and a second RNA fragment including a terminal region that includes a 3′ hydroxyl group, where the RNA is synthesized by ligating the first and second RNA fragments. When synthesizing a gRNA, the first RNA fragment, the second RNA fragment, or both, can include at least a portion of a sequence that can bind to an RNA-guided endonuclease. An exemplary mlRNA as synthesized by the methods described can include, from 5′ to 3′, the second RNA fragment followed by the first RNA fragment. The second RNA fragment may not include a 5′ phosphate moiety. The 5′ phosphate moiety can be, e.g., a 5′-phosphate or a 5′-phosphorothioate. The first fragment, the second RNA fragment, or both, can include a sequence or a portion of a sequence that is complementary to a sequence in a target DNA. In some instances, the second RNA fragment comprises a sequence that is complementary to a sequence in a target DNA.

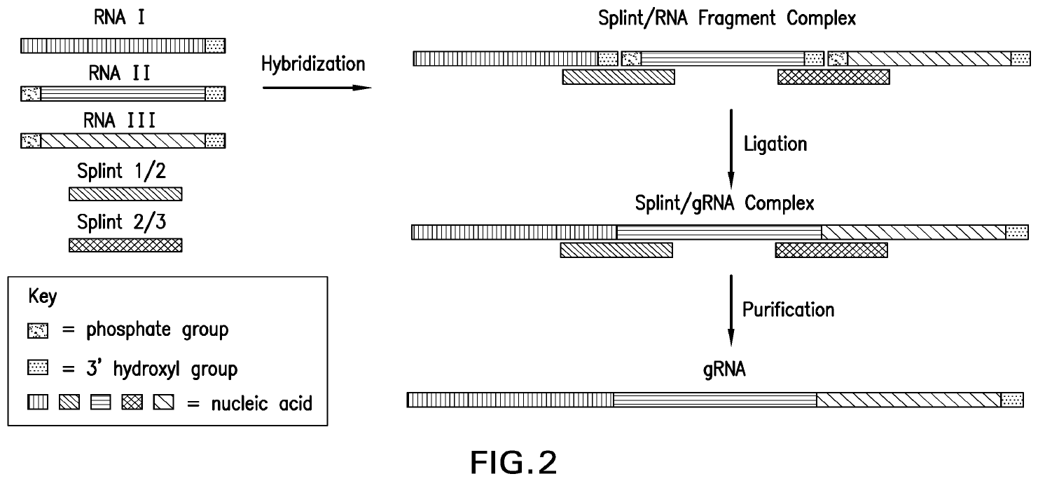

The mlRNAs can be synthesized by ligating three or more (e.g., three, four, five, or six) RNA fragments. FIG. 2 is a schematic diagram showing the ligation of three RNA fragments using two splint oligonucleotides. In some instances, the RNA is synthesized by ligating fewer than six RNA fragments. By way of illustration, for an RNA synthesized by ligating RNA fragments A, B and C (listed in a 5′ to 3′ order), prior to ligation, RNA fragment A can include a 3′ hydroxyl group and may not include a 5′ phosphate moiety, RNA fragment B can include both a 3′ hydroxyl group and a 5′ phosphate moiety, and RNA fragment C can include a 5′ phosphate moiety and may or may not include a 3′ hydroxyl group. Ligating RNA fragments A, B, and C can include formation of a phosphodiester bond between the 3′ hydroxyl group of A and the 5′ phosphate moiety of B, and a phosphodiester bond between the 3′ hydroxyl group of B and the 5′ phosphate moiety of C.

The length of any of the RNA fragments can be 10 to 90 nucleotides (e.g., 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 15, 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, 15 to 25, 15 to 20, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 90, 35 to 85, 35 to 80, 35 to 75, 35 to 70, 35 to 65, 35 to 60, 35 to 55, 35 to 50, 35 to 45, 35 to 40, 40 to 90, 40 to 85, 40 to 80, 40 to 75, 40 to 70, 40 to 65, 40 to 60, 40 to 55, 40 to 50, 40 to 45, 45 to 90, 45 to 85, 45 to 80, 45 to 75, 45 to 70, 45 to 65, 45 to 60, 45 to 55, 45 to 50, 50 to 90, 50 to 85, 50 to 80, 50 to 75, 50 to 70, 50 to 65, 50 to 60, 50 to 55, 55 to 90, 55 to 85, 55 to 80, 55 to 75, 55 to 70, 55 to 65, 55 to 60, 60 to 90, 60 to 85, 60 to 80, 60 to 75, 60 to 70, 60 to 65, 65 to 90, 65 to 85, 65 to 80, 65 to 75, 65 to 70, 70 to 90, 70 to 85, 70 to 80, 70 to 75, 75 to 90, 75 to 85, 75 to 80, 80 to 90, 80 to 85, or 85 to 90 nucleotides). The length of the first and second RNA fragments can be, for example, 10 to 90 nucleotides, each. The length of the second RNA fragment can be about 40 nucleotides or less (e.g. about 35, 30, 25, 20, 15, or about 10 nucleotides). The length of the second RNA fragment can be about 20 nucleotides in some instances, while the length of the first RNA fragment can be about 80 nucleotides.

An RNA fragment can include one or more secondary structures. The secondary structure of an RNA molecule (e.g., an RNA fragment or an mlRNA) can include stems and loops, or combination thereof. Non-limiting examples of secondary structures of an RNA molecule include stem loops, hairpin, hairpin loops, tetraloops, internal loops, bulges, pseudoknots, and cloverleaf. In some instances, an RNA fragment does not include any secondary structures (e.g., stem loops). The RNA synthesized by the methods provided herein can include one or more secondary structures, such as but not limited to, one or more stem loop structures, formed upon ligation of the RNA fragments. In some instances, the ligation site present between the RNA fragments correspond to a site in a secondary structure (e.g., a stem loop structure) in the synthesized RNA. The ligation site may correspond to a site in a portion of the secondary structure, including but not limited to, a tetraloop portion or a helix portion of a stem loop structure.

The methods of the present disclosure can include predicting the secondary structure(s) of an RNA fragment and/or the free energy associated with the secondary structure(s), based on the sequence of the RNA fragment. Methods of predicting RNA secondary structures are known in the art, including those described in Zuker and Stiegler (1981) Nucleic Acids Research, 9(1): 133-148, Reuter and Mathews (2010) BMC Bioinformatics 11:129; and Xia et al. (1998) Biochemistry, 37:14719-14735. RNA secondary structure can be predicted from the sequence of the RNA by free energy minimization, such as those described in Mathews and Turner (2006) Current Opinion in Structural Biology, 16:270-278. Non-limiting examples of software for RNA secondary structure prediction can be found at: available from URL: en.wikipedia.org/wiki/List_of_RNA_structure_prediction_software.

### Modifications

An RNA fragment can include one or more modifications. For example, an RNA fragment may include at least one modification in the RNA backbone. Non-limiting examples of backbone modifications include: 2′ methoxy (2′ OMe), 2′ fluorine (2′fluoro), 2′-O-methoxy-ethyl (MOE), locked nucleic acids (LNA), unlocked nucleic acids (UNA), bridged nucleic acids, 2′deoxynucleic acids (DNA), and peptide nucleic acids (PNA). Alternatively or additionally, an RNA fragment can include at least one base modification. Non-limiting examples of base modifications include: 2-aminopurine, inosine, thymine, 2,6-diaminopurine, 2-pyrimidinone, and 5-methyl cytosine. In some instances, an RNA fragment comprises at least one phosphorothioate linkage.

Modifications in the RNA fragments can be used to, e.g., enhance stability, reduce the likelihood or degree of innate immune response, and/or enhance other attributes; and new types of modifications are regularly being developed. By way of illustration of various types of modifications, modifications can include one or more nucleotides modified at the 2′ position of the sugar, such as but not limited to, a 2′-O-alkyl, 2′-O-alkyl-O-alkyl, or 2′-fluoro-modified nucleotide. DNA (2′deoxy-) nucleotide substitutions are also contemplated. Non-limiting examples of RNA modifications also include 2′-fluoro, 2′-amino, 2′ O-methyl modifications on the ribose of pyrimidines, and basic residues or an inverted base at the 3′ end of the RNA. Such modifications can be incorporated into oligonucleotides, and these oligonucleotides have been shown to have a higher Tₘ (e.g., higher target binding affinity) than 2′-deoxy oligonucleotides against a given target. In some embodiments, the modification of an RNA fragment disclosed herein comprises a 2′O-methyl modification of one or more nucleosides in the RNA fragment.

An RNA fragment according to any of the embodiments described herein can include, for example, a modification that increases resistance to nuclease digestion as compared to the native nucleic acid. In some instances, the modified nucleic acid comprises a modified backbone selected from, for example, phosphorothioates, phosphotriesters, methyl phosphonates, short chain alkyl or cycloalkyl intersugar linkages, and short chain heteroatomic or heterocyclic intersugar linkages. The nucleic acid can have a phosphorothioate backbone or a heteroatom backbone, e.g., CH₂—NH—O—CH₂, CH, —N(CH₃)—O—CH₂ (known as a methylene(methylimino) or MMI backbone), CH₂—O—N (CH₃)—CH₂, CH₂—N(CH₃)—N(CH₃)—CH₂ and O—N(CH₃)—CH₂—CH₂ backbones; amide backbones (see De Mesmaeker et al. (1995) Acc. Chem. Res., 28(9):366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al. (1991) Science, 254(5037):1497-1500). Phosphorus-containing linkages include, but are not limited to, phosphorothioates, chiral phosphorothioates, phosphorodithioates, phosphotriesters, aminoalkylphosphotriesters, methyl and other alkyl phosphonates including 3′alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see, e.g., U.S. Pat. Nos. 3,687,808; 4,469,863; 4,476,301; 5,023,243; 5,177, 196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050. In some embodiments, the modification of an RNA fragment disclosed herein comprises one or more phosphorotioate linkages in the backbone of the RNA fragment.

Morpholino-based oligomeric compounds are described in Braasch et al. (2002) Biochem., 4/(14):4503-4510; Genesis, Volume 30, Issue 3, (2001) Wiley Online Library; Heasman (2002) Dev. Biol., 243(2):209-214; Nasevicius et al. (2000) Nat. Genet., 26(2):216-220; Lacerra et al. (2000) Proc. Natl. Acad. Sci. USA, 97(17):9591-9591; and U.S. Pat. No. 5,034,506. Cyclohexenyl nucleic acid oligonucleotide mimetics are described in Wang et al. (2000) J. Am. Chem. Soc., 122(36):8595-8602.

An RNA fragment according to any of the embodiments described herein can include a backbone that does not include a phosphorus atom, e.g., backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see, e.g., U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

An RNA fragment according to any of the embodiments described herein can include one or more substituted sugar moieties including, e.g., one of the following at the 2′ position: OH, SH, SCH₃, F, OCN, OCH₃, OCH₃O(CH₂)ₙCH₃, O(CH₂), NH₂, or O(CH₂), CH₃, where n is from 1 to 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. For example, a modification can include 2′-methoxyethoxy (2′-O—CH₂CH₂OCH₃, also known as 2′-O-(2-methoxyethyl)) (Martin et al. (1995) Helv. Chico. Acta, 78(2):486-504). Other modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group. In some instances, both a sugar and an internucleoside linkage, e.g., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teachings of PNA compounds can be found in Nielsen et al. (1991) Science, 254(5037):1497-1500. An RNA fragment as described herein can include 2′-O-thionocarbamates MP (2′-O-methyl-3′-phosphonoacetate) and MSP (O-methyl-3′-thiophosphonoacetate) (Ryan et al., (2017) Nuc. Acids Res. 46 (2):792-803).

An RNA fragment as described herein can include one or more modifications selected from the group consisting of: pseudouridine, N¹-methylpseudouridine, and 5-methoxyuridine. For example, one or more N¹-methylpseudouridines can be incorporated into the RNA fragment to provide enhanced RNA stability and reduced immunogenicity in animal cells, such as mammalian cells (e.g., cells of human and mice). N¹-methylpseudouridine modifications can also be incorporated in combination with one or more 5-methylcytidines.

There are numerous commercial suppliers of modified RNAs, including, for example, Trilink Biotech, Axolabs, Bio-Synthesis Inc., and Dharmacon. As described by Trilink, for example, 5-Methyl-CTP can be used to impart desirable characteristics such as increased nuclease stability or reduced interaction of innate immune receptors with in vitro transcribed RNA. 5′-Methylcytidine-5′-triphosphate (5-methyl-CTP), N6-methyl-ATP, as well as pseudo-UTP and 2-thio-UTP, have also been shown to reduce innate immune stimulation in culture and in vivo as illustrated in Kormann et al. (2011) Nat. Biotechnol., 29:154-157 and Warren et al. (2010) Cell Stem Cell, 7(5):618-630.

An RNA fragment can incorporate modifications designed to bypass innate antiviral responses. See, e.g., Warren et al. (2010) Cell Stem Cell, 7(5):618-630. For example, the RNA can be an enzymatically synthesized RNA incorporating 5-methyl-CTP, pseudo-UTP, and/or an Anti-Reverse Cap Analog (ARCA); see, e.g. Warren et al. (2010) Cell Stem Cell, 7(5):618-630.

A large variety of modifications have been developed and applied to enhance RNA stability, reduce innate immune responses, and/or achieve other benefits; see, e.g., the reviews by Whitehead et al. (2011) Ann. Rev. Chem. Biomolec. Eng., 2:77-96; Gaglione et al. (2010) Mini Rev. Med. Chem., 10(7):578-595; Chernolovskaya et al. (2010) Curr. Opin. Mol. Ther., 12(2):158-167; Deleavey et al. (2009) Curr. Protoc. Nucleic Acid Chem., 39(1):16.3.1-16.3.22; Behlke (2008) Oligonucleotides, 18(4):305-319; Fucini et al. (2012) Nucleic Acid Ther., 22(3):205-210; Bremsen et al. (2012) Front. Genet., 3:154.

### Mimetics

An RNA fragment can be a nucleic acid mimetic. The term “mimetic” as it is applied to polynucleotides is intended to include polynucleotides wherein only the furanose ring or both the furanose ring and the internucleotide linkage are replaced with non-furanose groups. Replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone. Representative US patents that describe the preparation of PNA compounds include, but are not limited to: U.S. Pat. Nos. 5,539,082; 5,714,331; and 5,719,262. In some instances, an RNA fragment as described herein is a PNA.

An RNA fragment can be a polynucleotide mimetic based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. Morpholino-based polynucleotides are nonionic mimics of oligonucleotides, which are less likely to form undesired interactions with cellular proteins (Braasch et al. (2002) Biochemistry, 41(14): 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

An RNA fragment can be a polynucleotide mimetic referred to as cyclohexenyl nucleic acid (GeNA), where the furanose ring normally present in a DNA/RNA molecule is replaced with a cydohexenyl ring. GeNA DMT protected phosphoramidite monomers have been prepared and used for oligomeric compound synthesis following classical phosphoramidite chemistry. Fully modified GeNA oligomeric compounds and oligonucleotides having specific positions modified with GeNA have been prepared and studied (see Wang et al. (2000) J. Am. Chem. Soc., 122(36):8595-8602). The study of incorporating GeNA structures into natural nucleic acid structures was shown by NMR and circular dichroism to proceed with easy conformational adaptation.

An RNA fragment can be a Locked Nucleic Acid (LNA), in which the 2′-hydroxyl group is linked to the 4′ carbon atom of the sugar ring, forming a 2′-C,4′-C-oxymethylene linkage, thereby forming a bicyclic sugar moiety. The linkage can be a methylene (—CH2-)ₙ group bridging the 2′ oxygen atom and the 4′ carbon atom wherein n is 1 or 2 (Singh et al. (1998) Chem. Commun., 4:455-456). LNA and LNA analogs display very high duplex thermal stabilities with complementary DNA and RNA (Tₘ=+3 to +10° C.), stability towards 3′-exonucleolytic degradation, and good solubility properties. Potent and nontoxic antisense oligonucleotides containing LNAs have been described (Wahlestedt et al. (2000) Proc. Natl. Acad. Sci. U.S.A, 97(10):5633-5638). The synthesis and preparation of the LNA monomers adenine, cytosine, guanine, 5-methyl-cytosine, thymine and uracil, along with their oligomerization, and nucleic acid recognition properties have been described (Koshkin et al. (1998) Tetrahedron, 54(14):3607-3630). LNAs and preparation thereof are also described in WO 98/39352 and WO 99/14226.

### Modified Sugar Moieties

An RNA fragment can include one or more substituted sugar moieties including, for example, a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH₂)ₙO)ₘCH₃, O(CH₂)ₙOCH₃, O(CH₂)ₙNH₂, O(CH₂)CH₃, O(CH₂)ₙONH₂, and O(CH₂)ₙON((CH₂)ₙCH₃)₂, where n and m are from 1 to about 10. Other RNA fragments include a suitable sugar substituent group selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, a group for improving the pharmacodynamic properties of an oligonucleotide, and other substituents having similar properties. A suitable modification includes 2′-methoxyethoxy 2′-O—CH₂—CH₂OCH₃, also known as -2′-O-(2-methoxyethyl) or 2′-MOE) (Martin et al. (1995) Helv. Chim. Acta, 78(2):486-504) e.g., an alkoxyalkoxy group. A further suitable modification includes 2′-dimethylaminooxyethoxy, e.g., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), e.g., 2′-O—CH₂—O—CH₂—N(CH₃)₂.

Other suitable sugar substituent groups include methoxy (—O—CH₃), aminopropoxy (—O—CH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

An RNA fragment according to any of the embodiments described herein can include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine (see, Kornberg et al. (1980) DNA Replication (2^{nd} ed.) (pp. 75-77). San Francisco, Calif.: W. H. Freeman & Co.; Gebeyehu et al. (1987) Nucl. Acids Res., 15(11):4513-4534). A “universal” base known in the art, e.g. inosine, can also be included. 5-Me-C substitutions have been shown to increase nucleic acid duplex stability by 0.6-1.2° C. (Sanghvi (1993). Antisense Research and Applications, (pp. 276-278). Crooke, S. T. and Lebleu, B., (Eds.), Boca Raton, Fla.: CRC Press) and are embodiments of base substitutions.

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other a-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases include those disclosed in U.S. Pat. No. 3,687,808, those disclosed in Kroschwitz (Ed.) (1990). The Concise Encyclopedia of Polymer Science and Engineering, (pp. 858-859). Hoboken, N.J.: John Wiley & Sons, those disclosed by Englisch et al. (1991) Angewandte Chemie International Edition, 30(6):613-722, and those disclosed by Sanghvi (1993) Chapter 15, Antisense Research and Applications, (pp. 289-302), Crooke, S. T. and Lebleu, B. (Eds), Boca Raton, Fla.: CRC Press. Certain of these nucleobases are particularly useful for increasing the binding affinity of the oligomeric compounds of the disclosure. These include 5-substituted pyrimidines, 6-azapyrimidines and N-2, N-6 and —O-6 substituted purines, including 2-aminopropyladenine, 5-propynyluracil and 5-propynylcytosine. 5-methylcytosine substitutions have been shown to increase nucleic acid duplex stability by 0.6-1.2 oc (Sanghvi (1993) Antisense Research and Applications, (pp. 276-278). Crooke and Lebleu, (Eds.), Boca Raton, Fla.: CRC Press) and are embodiments of base substitutions, even more particularly when combined with 2′-0-methoxyethyl sugar modifications. Modified nucleobases are described, e.g., in U.S. Pat. No. 3,687,808, as well as U.S. Pat. Nos. 4,845,205; 5,130,302; 5,134,066; 5,175, 273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and US Publication No. 2003/0158403.

An RNA fragment according to any of the embodiments described herein comprising nucleobase modifications or substitutions may not have all positions uniformly modified. For example, an RNA fragment may have a modification incorporated in a single nucleoside.

### IV. Synthesis of RNA Fragments and Splint Oligonucleotides

The RNA fragments and splint oligonucleotides provided by the present disclosure may be synthesized by any method suitable for oligonucleotide synthesis described herein or known in the art. Non-limiting examples include enzymatic synthesis and chemical synthesis (e.g., phosphoramidite chemistry).

Methods of synthesizing RNA from a DNA template are known in the art. For example, the RNA fragments and splint oligonucleotides can be synthesized in vitro using an RNA polymerase enzyme (e.g., T7 polymerase, T3 polymerase, SP6 polymerase, etc.). Solid-phase synthesis using phosphoramidite chemistry involves assembling monomers of protected 2′-deoxynucleosides (dA, dC, dG, and T), ribonucleoside (A, C, G, and US), or chemically modified nucleosides, e.g., LNA or BNA. The monomers are sequentially coupled to the growing oligonucleotide chain in the order required by the sequence of the product. Upon the completion of the chain assembly, the product is released from the solid phase to solution, deprotected, and collected.

The RNA fragments and splint oligonucleotides can be synthesized in a 5′ to 3′ direction or a 3′ to 5′ direction. In some instances, the second RNA fragment is synthesized in a 5′ to 3′ direction. The synthesized RNA fragments and splint oligonucleotides may be purified prior to ligation in accordance with known methods in the art, such as, but not limited to: high-performance liquid chromatography (HPLC), reversed-phase HPLC, ion-exchange chromatography, size exclusion chromatography, hydrophobic interaction chromatography, affinity chromatography, and polyacrylamide gel purification.

### V. Moderate Length RNAs

An exemplary mlRNA synthesized using any of the methods described herein is a guide RNA (gRNA) (e.g., any of the gRNAs described herein). A gRNA synthesized by the methods of the present disclosure can be a single-molecule gRNA (sgRNA) or double-molecule gRNA. A gRNA provides target specificity by virtue of its association with the RNA-guided endonuclease, and thus directs the activity of the RNA-guided endonuclease. RNAs of the present disclosure can be synthesized from two or more RNA molecules (termed RNA fragments) using one or more splints. An exemplary double-molecule gRNA comprises a crRNA and a transactivating crRNA (tracrRNA), and the crRNA and tracrRNA hybridize to each other to form a duplex. A double-molecule gRNA can also be a duplex of two crRNAs. The gRNA duplex can bind a RNA-guided endonuclease such that the gRNA and the RNA-guided endonuclease form a complex. A crRNA comprises both a spacer sequence capable of hybridizing to a target nucleic acid sequence of interest and a crRNA repeat sequence. TracrRNAs can be in any form (e.g., full-length tracrRNAs or active partial tracrRNAs) and of varying lengths. For example, a tracrRNA may comprise or consist of all or a portion of a wild-type tracrRNA sequence (e.g., about or at least 20, 26, 32, 45, 48, 54, 63, 67, 85 or more nucleotides of a wild-type tracrRNA sequence). Examples of wild-type tracrRNA sequences from S. pyogenes include the 171-nucleotide, 89-nucleotide, 75-nucleotide, and 65-nucleotide versions. See, for example, Deltcheva et al. (2011) Nature 471:602-607 and WO 2014/093661. As an example, the crRNA can have, in the 5′ to 3′ direction, an optional spacer extension sequence, a spacer sequence, and a minimum CRISPR repeat sequence. The tracrRNA can have a minimum tracrRNA sequence (complementary to the minimum CRISPR repeat sequence), a 3′ tracrRNA sequence, and an optional tracrRNA extension sequence. The optional tracrRNA extension may have elements that contribute additional functionality (e.g., stability) to the gRNA, and can have one or more hairpin structures. The crRNA and the tracrRNA hybridize through the minimum CRISPR repeat sequence and the minimum tracrRNA sequence to form a gRNA.

An exemplary sgRNA comprises a nucleotide sequence that is complementary to a sequence in a target DNA, and a nucleotide sequence that can bind to an RNA-guided endonuclease. As an example, an sgRNA can have, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence, and an optional tracrRNA extension sequence. In some instances, an sgRNA can have, in the 5′ to 3′ direction, a minimum CRISPR repeat sequence and a spacer sequence. The single-molecule guide linker links the minimum CRISPR repeat and the minimum tracrRNA sequence to form a hairpin structure. In some embodiments, the single-molecule guide linker is a tetraloop. Exemplary gRNAs are described, for example, in WO 2018/002719.

In general, a CRISPR repeat sequence includes any sequence that has sufficient complementarity with a tracr sequence to promote one or more of: (1) excision of a DNA targeting segment flanked by CRISPR repeat sequences in a cell containing the corresponding tracr sequence; and (2) formation of a CRISPR complex at a target sequence, wherein the CRISPR complex includes the CRISPR repeat sequence hybridized to the tracr sequence. In general, degree of complementarity is with reference to the optimal alignment of the CRISPR repeat sequence and tracr sequence, along the length of the shorter of the two sequences. Optimal alignment may be determined by any suitable alignment algorithm and may further account for secondary structures, such as self-complementarity within either the tracr sequence or CRISPR repeat sequence. In some instances, the degree of complementarity between the tracr sequence and CRISPR repeat sequence along the 30 nucleotides length of the shorter of the two when optimally aligned is about or more than 25%, 30%, 40%, 50%, 60%, 70%, 80%, 90%, 95%, 97.5%, 99%, or higher. The tracr sequence can be, e.g., about or more than 5, 6, 7, 8, 9, 10, 11, 12, 13, 14, 15, 16, 17, 18, 19, 20, 25, 30, 40, 50, or more nucleotides in length.

The spacer of a gRNA includes a nucleotide sequence that is complementary to a sequence in a target DNA. In other words, the spacer of a gRNA interacts with a target DNA in a sequence-specific manner via hybridization (e.g., base pairing). As such, the nucleotide sequence of the spacer may vary and determines the location within the target DNA that the gRNA and the target DNA will interact. The spacer of a gRNA can be selected to hybridize to any desired sequence within a target DNA.

The spacer can have a length of, e.g., from 10 nucleotides to 30 nucleotides (such as a length of any of 10, 11, 12, 13, 14, 15, 16, 17, 18, 19, 20, 21, 22, 23, 24, 25, 26, 27, 28, 29, or 30 nucleotides). For example, the spacer can have a length of from 13 nucleotides to 25 nucleotides, from 15 nucleotides to 23 nucleotides, from 18 nucleotides to 22 nucleotides, or from 20 nucleotides to 22 nucleotides.

The percent sequence complementarity between the spacer of a gRNA and a target sequence of a target DNA can be, e.g., at least about 60% (such as at least about any of 65%, 70%, 75%, 80%, 85%, 90%, 95%, 97%, 98%, 99%, or 100%).

The length of the gRNA synthesized by the methods described herein can be, e.g., from 30 to 160 nucleotides, e.g., from 40 to 150, from 50 to 140, from 60 to 130, from 70 to 120, from 80 to 110, or from 90 to 100 nucleotides, (such as a length of any of 35, 40, 45, 50, 55, 60, 65, 70, 75, 80, 85, 90, 95, 100, 105, 110, 115, 120, 125, 130, 135, 140, 145, or 150 nucleotides). The gRNA synthesized by the methods described herein can include a spacer. In some embodiments, the gRNA synthesized by the methods described herein includes a sequence that is complementary to a sequence in a target DNA, including but not limited to, a target mammalian DNA. For example, the target DNA can be human DNA.

Modifications in the miRNAs

mlRNAs as described herein can include one or more modifications useful for e.g., tracking, increasing stability, targeting the RNA to a particular subcellular location, or reducing immunogenicity. Modifications of gRNAs can be used to enhance the formation or stability of a DNA-editing complex comprising a gRNA and an RNA-guided endonuclease (e.g., a Cas endonuclease, such as a Cas9 endonuclease). Modifications of gRNAs can also or alternatively be used to enhance the initiation, stability, or kinetics of interactions between a DNA-editing complex and a target sequence in a target DNA, which can be used, for example, to enhance on-target activity. Modifications of gRNAs can also or alternatively be used to enhance specificity, e.g., the relative rates of DNA editing at an on-target site as compared to effects at other (off-target) sites.

Modifications can also or alternatively be used to increase the stability of an RNA, e.g., by increasing its resistance to degradation by ribonucleases (RNases) present in a cell, thereby causing its half-life in the cell to be increased.

mlRNAs according to any of the embodiments described herein can include a segment at either the 5′ or 3′ end that provides for any of the features described above. For example, a suitable segment can include a riboswitch sequence (e.g., to allow for regulated stability and/or regulated accessibility by proteins and protein complexes); a stability control sequence; a sequence that forms a dsRNA duplex (e.g., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides for responses to light or radiation (e.g., UV, vis, IR optogenetic elements); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); a modification or sequence that provides for increased, decreased, and/or controllable stability; and combinations thereof.

mlRNAs according to any of the embodiments described herein can include a modification that decreases the likelihood or degree to which the RNA, when introduced into a cell, elicits an innate immune response. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

mlRNAs according to any of the embodiments described herein can also include one or more modifications selected from modifications that enhance the stability of the RNA (such as by decreasing its degradation by RNases, e.g., in the context of a cell) and modifications that decrease the likelihood or degree to which the RNA, when introduced into a cell, elicits an innate immune response. Combinations of modifications, such as the foregoing and others, can likewise be used.

Stability Control Sequence mlRNAs according to any of the embodiments described herein can include a stability control sequence that influences the stability of the RNA. A non-limiting example of a suitable stability control sequence is a transcriptional terminator segment (e.g., a transcription termination sequence). A transcriptional terminator segment of an RNA can have a total length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the transcriptional terminator segment can have a length of from about 15 nucleotides (nt) to about 80 nt, from about 15 nt to about 50 nt, from about 15 nt to about 40 nt, from about 15 nt to about 30 nt or from about 15 nt to about 25 nt.

The transcription termination sequence can be one that is functional in a eukaryotic cell, and/or a prokaryotic cell.

Nucleotide sequences that can be included in a stability control sequence (e.g., transcriptional termination segment, or in any segment of the RNA to provide for increased stability) include, for example, a Rho-independent trp termination site.

Conjugates mlRNAs according to any of the embodiments described herein can include a modification involving chemically linking to the gRNA one or more moieties or conjugates which enhance the activity, cellular distribution, or cellular uptake of the RNA. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a nucleic acid.

mlRNAs according to any of the embodiments described herein can include a chemically linked conjugate moieties including, but not limited to, lipid moieties such as a cholesterol moiety (Letsinger et al. (1989) Proc. Natl. Acad. Sci. U.S.A., 86(17):6553-6556); cholic acid (Manoharan et al. (1994) Bioorg. Med. Chem. Let., 4(8):1053-1060); a thioether, e.g., hexyl-S-tritylthiol (Manoharan et al. (1992). Ann. N. Y. Acad. Sci., 660(1):306-309; and Manoharan et al. (1993) Bioorg. Med. Chem. Let., 3(12):2765-2770); a thiocholesterol (Oberhauser et al. (1992) Nucl. Acids Res., 20(3):533-538); an aliphatic chain, e.g., dodecandiol or undecyl residues (Saison-Behmoaras et al. (1991) EMBO J., 10(5):1111-1118; (Kabanov et al. (1990) FEBS Lett., 259(2):327-330 and Svinarchuk et al. (1993) Biochimie, 75(1-2):49-54), a phospholipid, e.g. di-hexadecyl-rac-glycerol or triethylammonium 1,2-di-O-hexadecyl-rac-glycero-3-H-phosphonate (Manoharan et al. (1995) Tetrahedron Lett., 36(20:3651-3654; Shea et al. (1990) Nucl. Acids Res., 18(13):3777-3783)), a polyamine or a polyethylene glycol chain (Manohoran et al. (1995) Nucleos. Nucleot. Nucl., 14(3-5): 969-973); adamantane acetic acid (Manoharan et al. (1995) Tetrahedron Lett., 36(20:3651-3654); a palmityl moiety (Mishra et al. (1995) Biochim. Biophys. Acta, 1264(2):229-237); or an octadecylamine or hexylamino-carbonyl-t oxycholesterol moiety (Crooke et al. (1996) J. Pharmacol. Exp. Ther., 277(2):923-937).

mlRNAs according to any of the embodiments described herein can include a chemically linked conjugate including a “Protein Transduction Domain” or PTD (also known as a cell penetrating peptide, or CPP), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. A PTD can be covalently linked to a gRNA. Exemplary PTDs include, but are not limited to, a minimal undecapeptide protein transduction domain (corresponding to residues 47-57 of HIV-1 TAT comprising YGRKKRRQRRR (SEQ ID NO: 1); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther., 9(6):489-496); a Drosophila antennapedia protein transduction domain (Noguchi et al. (2003) Diabetes, 52(7):1732-1737); a truncated human calcitonin peptide (Tréhin et al. (2004) Pharm. Research, 21(7):1248-1256); polylysine (Wender et al. (2000) Proc. Natl. Acad. Sci. USA, 97(24):13003-13008). The PTD can be an activatable CPP (ACPP) (Aguilera et al. (2009) Integr. Biol. (Camb), 1(5-6):371-381). ACPPs include a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane. The PTD can be chemically modified to increase the bioavailability of the PTD. Exemplary modifications are disclosed in Mae et al. (2009) Expert Opin. Drug Deliv., 6(11): 1195-1205.

mlRNAs according to any of the embodiments described herein can also include an applied conjugate that can enhance its delivery and/or uptake by cells, including, for example, cholesterol, tocopherol and folic acid, lipids, peptides, polymers, linkers, and aptamers; see, e.g., the review by Winkler (2013) Ther. Deliv., 4(7):791-809, and references cited therein.

### VI. RNA-Guided Endonucleases

The presently disclosed methods of synthesizing a gRNA generally include providing a first RNA fragment, a second RNA fragment, where the first RNA fragment, the second RNA fragment, or both, comprises at least a portion of a sequence that can bind to an RNA-guided endonuclease.

The RNA-guided endonuclease can be naturally-occurring or non-naturally occurring. Examples of such endonucleases include a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, and functional derivatives thereof. In some instances, the RNA-guided endonuclease is a Cas9 endonuclease. The Cas9 endonuclease can be from, e.g., Streptococcus pyogenes (SpyCas9), Staphylococcus lugdunensis (SluCas9), or from Staphylococcus aureus (SaCas9). In some instances, the RNA-guided endonuclease is a variant of Cas9, and the variant of Cas9 is selected from the group consisting of: a small Cas9, a dead Cas9 (dCas9), and a Cas9 nickase.

The RNA-guided endonuclease can be a small RNA-guided endonuclease. The small RNA-guided endonucleases can be engineered from portions of RNA-guided endonucleases derived from any of the RNA-guided endonucleases described herein and known in the art. The small RNA-guided endonucleases can be, e.g., small Cas endonucleases (e.g., PCT/US2018/065863; PCT/US2019/023044). In some cases, a small RNA-guided nuclease is, e.g., smaller than about 1,100 amino acids in length.

The RNA-guided endonuclease can be a mutant RNA-guided endonuclease. For example, the RNA-guided endonuclease can be a mutant of a naturally occurring RNA-guided endonuclease. The mutant RNA-guided endonuclease can also be a mutant RNA-guided endonuclease with altered activity compared to a naturally occurring RNA-guided endonuclease, such as altered endonuclease activity (e.g., altered or abrogated DNA endonuclease activity without substantially diminished binding affinity to DNA). Such modification can allow for the sequence-specific DNA targeting of the mutant RNA-guided endonuclease for the purpose of transcriptional modulation (e.g., activation or repression); epigenetic modification or chromatin modification by methylation, demethylation, acetylation or deacetylation, or any other modifications of DNA binding and/or DNA-modifying proteins known in the art. In some instances, the mutant RNA-guided endonuclease has no DNA endonuclease activity.

The RNA-guided endonuclease can be a nickase that cleaves the complementary strand of the target DNA but has reduced ability to cleave the non-complementary strand of the target DNA, or that cleaves the non-complementary strand of the target DNA but has reduced ability to cleave the complementary strand of the target DNA. In some instances, the RNA-guided endonuclease has a reduced ability to cleave both the complementary and the non-complementary strands of the target DNA.

VII. Methods of Synthesizing sgRNAs

In some embodiments, the disclosure provides methods for synthesizing a sgRNA for use with an RNA-guided endonuclease. In some embodiments, the RNA-guided endonuclease is a Cas endonuclease. In some embodiments, the RNA-guided endonuclease is a Cas9 endonuclease. In some embodiments, the Cas9 endonuclease is a SpyCas9, SaCas9, or SluCas9 endonuclease. In some embodiments, the RNA-endonuclease is a Cas9 variant. In some embodiments, the RNA-guided endonuclease is a small RNA-guided endonuclease. In some embodiments, the RNA-guided endonuclease is a small Cas endonuclease.

In some embodiments, the sgRNA comprise 5′ to 3′: a crRNA and a tracrRNA, wherein the crRNA and tracrRNA hybridize to form a duplex. In some embodiments, the crRNA comprises a spacer sequence capable of targeting a target sequence in a target nucleic acid (e.g., genomic DNA molecule) and a crRNA repeat sequence. In some embodiments, the tracrRNA comprises a tracrRNA anti-repeat sequence and a 3′ tracrRNA sequence. In some embodiments, the 3′ end of the crRNA repeat sequence is linked to the 5′ end of the tracrRNA anti-repeat sequence, e.g., by a tetraloop, wherein the crRNA repeat sequence and the tracrRNA anti-repeat sequence hybridize to form the sgRNA. In some embodiments, the sgRNA comprises 5′ to 3′: a spacer sequence, a crRNA repeat sequence, a tetraloop, a tracrRNA anti-repeat sequence, and a 3′ tracrRNA sequence. In some embodiments, the sgRNA further comprise a 5′ spacer extension sequence. In some embodiments, the sgRNA further comprise a 3′ tracrRNA extension sequence. In some embodiments, the 3′ tracrRNA comprises one or more stem loops. In some embodiments, the 3′ tracRNA comprises one, two, three, or more stem loops. In some embodiments, the 3′ tracrRNA consists of one, two, or three stem loops.

In some embodiments, the method comprises synthesizing the sgRNA using a splint-mediated ligation approach comprising two RNA fragments and one splint oligonucleotide. In some embodiments, the method comprises providing a complex formed between a first RNA fragment, a second RNA, and a splint oligonucleotide; and a ligase, wherein (a) the first RNA fragment comprises a terminal region comprising a 3′ hydroxyl group; (b) the second RNA fragment comprises a terminal region comprising a 5′ phosphate moiety; and (c) the splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and wherein the complex is formed by hybridization of (a) and (c)(i) and hybridization of (b) and (c)(ii), wherein the complex comprises a ligation site present between the 3′ hydroxyl group of the first RNA fragment and the 5′ phosphate group of the second RNA fragment, wherein the ligase results in a ligation at the ligation site to form a phosphodiester bond between the 3′hydroxyl group of the first RNA fragment and the 5′ phosphate group of the second RNA fragment, the ligation forming a sgRNA comprising from 5′ to 3′: a spacer sequence and an invariable sequence, the invariable sequence comprising a duplex formed between a crRNA repeat sequence and a tracrRNA anti-repeat sequence, and a 3′ tracrRNA sequence comprising at least one stem-loop, thereby synthesizing the sgRNA. In some embodiments, the ligation site corresponds to a site in the duplex formed between a crRNA repeat sequence and a tracrRNA anti-repeat sequence. In some embodiments, the ligation site is in the crRNA repeat sequence, in a tetraloop joining the crRNA repeat sequence and the tracRNA anti-repeat sequence, or in the tracrRNA anti-repeat sequence. In some embodiments, the ligation site is within a stem-loop of the 3′ tracrRNA sequence. In some embodiments, the first RNA fragment comprises the nucleotide sequence of the sgRNA that is 5′ the ligation site; and the second RNA fragment comprises the nucleotide sequence of the sgRNA that is 3′ the ligation site.

In some embodiments, the method comprises synthesizing a sgRNA using a splint-mediated ligation approach comprising three RNA fragments and two splint oligonucleotides. In some embodiments, the method comprises providing a complex formed between a first RNA fragment, a second RNA fragment, a third RNA fragment, first splint oligonucleotide, and a second splint oligonucleotide; and a ligase, wherein (a) the first RNA fragment comprises (i) a terminal region comprising a 3′ hydroxyl group; (b) the second RNA fragment comprises (i) a first terminal region comprising a 5′ phosphate moiety, and (ii) a second terminal region comprising a 3′ hydroxyl group; (c) the third RNA fragment comprises (i) a terminal region comprising a 5′ phosphate moiety; (d) the first splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and (e) the second splint oligonucleotide comprises (i) a first portion complementary to the second terminal region comprising the 3′ hydroxyl group of the second RNA fragment, and (ii) a second portion complementary to the terminal region comprising the 5′ phosphate moiety of the third RNA fragment, wherein the complex is formed by hybridization of (a)(i) and (d)(i), (b)(i) and (d)(ii), (b)(ii) and (e)(i), and (c)(i) and (e)(ii), wherein the complex has a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment, wherein the ligase results in a ligation at the first ligation site, forming a phosphodiester bond between the 3′ hydroxyl group of the first RNA fragment and the 5′ phosphate group of the second RNA fragment, and a ligation at the second ligation site, forming a phosphodiester bond between the 3′ hydroxyl group of the second RNA fragment and the 5′ phosphate group of the third RNA fragment, the ligation forming a sgRNA comprising from 5′ to 3′: a spacer sequence and an invariable sequence, the invariable sequence comprising a duplex formed between a crRNA repeat sequence and a tracrRNA anti-repeat sequence, and a 3′ tracrRNA sequence comprising at least one stem-loop, thereby synthesizing the sgRNA.

In some embodiments, the first ligation site corresponds to a site in the duplex formed between a crRNA repeat sequence and a tracrRNA anti-repeat sequence. In some embodiments, the first ligation site is in the crRNA repeat sequence, in a tetraloop joining the crRNA repeat sequence and the tracRNA anti-repeat sequence, or in the tracrRNA anti-repeat sequence.

In some embodiments, the 3′ tracrRNA sequence comprises a first, second, and third stem loop. In some embodiments, the second ligation site corresponds to a site in the first stem loop, the second stem loop, or the third stem loop. In some embodiments, the second ligation site corresponds to a site in the second stem loop. In some embodiments, the second ligation site corresponds to a site in the 5′ stem of the second stem loop, a site in the tetraloop of the second stem loop, or a site in the 3′ stem of the second stem loop. In some embodiments, the second ligation site is (i) immediately adjacent to the base of the 5′stem of the second stem loop; (ii) proximal to the base of the 5′stem of the second stem loop (e.g., ±1 nt, ±2 nt, or ±3 nt from the base of the 5′ stem); (iii) immediately adjacent to the base of the 3′stem of the second stem loop; or (iv) proximal to the base of the 3′stem of the second stem loop (e.g., ±1 nt, ±2 nt, or ±3 nt from the base of the 3′ stem).

In some embodiments, the first RNA fragment comprises the nucleotide sequence of the sgRNA that is 5′ the first ligation site; the second RNA fragment comprises the nucleotide sequence of the sgRNA that is 3′ the first ligation site and 5′ the second ligation site; and the third RNA fragment comprises the nucleotide sequence of the sgRNA that is 3′ the second ligation site.

In some embodiments, the first RNA fragment comprises 5′ to 3′: the spacer sequence, and a portion of the crRNA repeat sequence of the sgRNA. In some embodiments, the first RNA fragment comprises 5′ to 3′: the spacer sequence, and the crRNA repeat sequence of the sgRNA. In some embodiments, the first RNA fragment comprises 5′ to 3′: the spacer sequence, the crRNA repeat sequence, and a portion of the tetraloop of the sgRNA. In some embodiments, the first RNA fragment comprises 5′ to 3′: the spacer sequence, the crRNA repeat sequence, and the tetraloop of the sgRNA. In some embodiments, the first RNA fragment comprises 5′ to 3′: the spacer sequence, the crRNA repeat sequence, the tetraloop, and a portion of the tracRNA repeat sequence of the sgRNA. In some embodiments, the first RNA fragment comprises 5′ to 3′: the spacer sequence, the crRNA repeat sequence, the tetraloop, and the tracRNA repeat sequence of the sgRNA. In some embodiments, the terminal region of (a)(i), which is complementary to (d)(i) of the first splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, or 34 nucleotides in length, wherein the nucleotide sequence is positioned at the 3′end of the first RNA fragment. In some embodiments, the terminal region of (a)(i) extends from the 3′ terminus of the first RNA fragment to the 3′ terminus of the spacer sequence (e.g., wherein the 5′ terminus of the spacer sequence is aligned with the 5′ terminus of the first RNA fragment). In some embodiments, the terminal region of (a)(i) extends from the 3′ terminus of the first RNA fragment to include the 1, 2, 3, 4, 5, 6, 7, 8, 9, 10, 11, 12, 13, 14, 15, 16, 17, 18, 19, or 20 nucleotides that are present at the 3′ end of the spacer sequence. In some embodiments, the first portion (d)(i) of the first splint oligonucleotide is perfectly complementary to the terminal region of (a)(i). In some embodiments, the first portion (d)(1) of the first splint oligonucleotide has 1, 2, or 3 mismatches relative to the terminal region of (a)(i).

In some embodiments, the second RNA fragment comprises 5′ to 3′: the crRNA repeat sequence, the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: a portion of the crRNA repeat sequence, the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: a portion of the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: the tracrRNA repeat sequence, and a portion of the 3′tracrRNA (i.e., a portion that is 5′ to the second ligation site) of the sgRNA. In some embodiments, the second RNA fragment comprises 5′ to 3′: a portion of the tracrRNA repeat sequence, and a portion of the 3′tracrRNA (i.e., a portion that is 5′ to the second ligation site) of the sgRNA. In some embodiments, the terminal region (b)(i), which is complementary to the second portion (d)(ii) of the first splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, or 34 nucleotides in length and is located at the 5′end of the second RNA fragment. In some embodiments, the second portion (d)(ii) of the first splint oligonucleotide is perfectly complementary to the terminal region of (b)(i), or has 1, 2, or 3 mismatches relative to the terminal region of (d)(ii). In some embodiments, the terminal region (b)(ii) of the second RNA fragment, which is complementary to the first portion (e)(i) of the second splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, or 34 nucleotides in length and is located at the 3′end of the second RNA fragment. In some embodiments, the first portion (e)(i) of the second splint oligonucleotide is perfectly complementary to the terminal region of (b)(ii), or has 1, 2, or 3 mismatches relative to the terminal region of (b)(ii).

In some embodiments, the third RNA fragment comprises a portion of the 3′ tracrRNA sequence (i.e., that is 3′ to the second ligation site) of the sgRNA. In some embodiments, the terminal region (c)(i) of the third RNA fragment, which is complementary to the second portion (e)(ii) of the second splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length and is located at the 5′end of the third RNA fragment. In some embodiments, the second portion (e)(ii) of the second splint oligonucleotide is perfectly complementary to the terminal region of (c)(i), or has 1, 2, or 3 mismatches relative to the terminal region of (c)(i).

In some embodiments, the invariable sequence of the sgRNA comprises the nucleotide sequence of SEQ ID NO: 17, or a nucleotide sequence having up to 1, 2, 3, 4, 5, 6, 7, 8, 9, or 10 nucleotide deletions, insertions, or substitutions relative to SEQ ID NO: 17. In some embodiments, the sgRNA is for use with a SpyCas9 endonuclease, wherein the invariable sequence of the sgRNA comprises the nucleotide sequence of SEQ ID NO: 17, or a nucleotide sequence having up to 1, 2, 3, 4, 5, 6, 7, 8, 9, or 10 nucleotide deletions, insertions, or substitutions relative to SEQ ID NO: 17.

In some embodiments, the first RNA fragment, the second RNA fragment, and the third RNA fragment respectively are selected from the nucleotide sequences comprising:

(a)(i) N₁₅₋₃₀GUUUUAGAGCUAG (SEQ ID NO: 56), wherein N₁₅₋₃₀ corresponds to a spacer sequence targeting a target site in a target nucleic acid (e.g., genomic DNA molecule); (ii) SEQ ID NO: 3; and (iii) SEQ ID NO: 4;

(b)(i) N₁₅₋₃₀GUUUUAGAGCUAGA (SEQ ID NO: 57), wherein N₁₅₋₃₀ corresponds to a spacer sequence targeting a target site in a target nucleic acid (e.g., genomic DNA molecule); (ii) SEQ ID NO: 40; and (iii) SEQ ID NO: 42;

(c)(i) N₁₅₋₃₀GUUUUAGAGCUAG (SEQ ID NO: 56), wherein N₁₅₋₃₀ corresponds to a spacer sequence targeting a target site in a target nucleic acid (e.g., genomic DNA molecule); (ii) SEQ ID NO: 58; and (iii) SEQ ID NO: 42; or

(d)(i) N₁₅₋₃₀GUUUUAGAGCUAGA (SEQ ID NO: 57), wherein N₁₅₋₃₀ corresponds to a spacer sequence targeting a target site in a target nucleic acid (e.g., genomic DNA molecule); (ii) SEQ ID NO: 59; and (iii) SEQ ID NO: 4.

In some embodiments, the first splint oligonucleotide comprises the sequence set forth in SEQ ID NO: 60; SEQ ID NO: 44; or SEQ ID NO: 61. In some embodiments, the first portion (d)(i) of the first splint oligonucleotide comprises a sequence that is complementary to the terminal region (a)(i) of the first RNA fragment, wherein the terminal region extends from the 3′ terminus of the first RNA fragment to include the 1, 2, 3, 4, 5, 6, 7, 8, 9, 10, 11, 12, 13, 14, 15, 16, 17, 18, 19, or 20 nucleotides from the 3′terminus of the spacer sequence of the sgRNA, wherein the 5′terminus of the spacer sequence aligns with the 5′terminus of the first RNA fragment. In some embodiments, the first portion (d)(i) of the first splint oligonucleotide comprises a sequence that is complementary to the terminal region (a)(i) of the first RNA fragment, wherein the terminal region is immediately adjacent to or 1, 2, or 3 nt downstream the 3′terminus of the spacer sequence of the sgRNA, wherein the 5′terminus of the spacer sequence aligns with the 5′terminus of the first RNA fragment. In some embodiments, the second splint oligonucleotide comprises the nucleotide sequence set forth in SEQ ID NO: 6; SEQ ID NO: 45; or SEQ ID NO: 53.

In some embodiments, the first RNA fragment, the second RNA fragment, and the third RNA fragment are each independently about 10 to about 90 nucleotides, about 10 to about 60 nucleotides, about 10 to about 50 nucleotides, about 10 to about 40 nucleotides, about 20 to about 40 nucleotides, about 30 to about 40 nucleotides in length.

In some embodiments, the first splint oligonucleotide is a DNA oligonucleotide. In some embodiments, the first splint oligonucleotide is an RNA oligonucleotide. In some embodiments, the second splint oligonucleotide is a DNA oligonucleotide. In some embodiments, the second splint oligonucleotide is an RNA oligonucleotide. In some embodiments, the first splint oligonucleotide and the second splint oligonucleotide are each independently about 20 to about 100 nucleotides, about 20 to about 90 nucleotides, about 20 to about 80 nucleotides, about 20 to about 70 nucleotides, about 20 to about 60 nucleotides, about 30 to about 60 nucleotides, or about 30 to about 50 nucleotides in length.

In some embodiments, the first, second, and/or third RNA fragment(s) are synthesized according to a method described herein, e.g., in vitro using an RNA polymerase enzyme or using solid-phase synthesis using phosphoramidite chemistry. In some embodiments, the RNA fragment(s) are synthesized using phosphoramidite chemistry, wherein (i) synthesis of the first RNA fragment, synthesis of the second RNA fragment, and/or synthesis of the third RNA fragment each proceeds in a 5′ to 3′ or in a 3′ to 5′ direction; or (ii) synthesis of the first RNA fragment proceeds in a 5′ to 3′ or in a 3′ to 5′ direction and synthesis of the second RNA fragment and/or synthesis of the third RNA fragment each proceed in a 3′ to 5′ direction. In some embodiments, the RNA fragment(s) are purified following synthesis.

In some embodiments, the first and/or second splint oligonucleotide(s) are synthesized according to a method described herein, e.g., in vitro using an RNA polymerase enzyme or using solid-phase synthesis using phosphoramidite chemistry. In some embodiments, the splint oligonucleotide(s) are purified following synthesis.

In some embodiments, the first, second, and/or third RNA fragment(s) comprise one or more modifications of the RNA backbone described herein, e.g., a backbone linkage or nucleoside modification. In some embodiments, the modification is a phosphorothioate linkage. In some embodiments, the modification is a 2′-O-methylation of a nucleoside.

In some embodiments, the hybridization is performed according to a method described herein. In some embodiments, the hybridization is performed in a solution. In some embodiments, the hybridization is performed with or without an annealing step. In some embodiments, the annealing comprises (i) heating the solution to about 80° C. to about 95° C. for a period of time less than about 10 minutes (e.g., 1, 2, 3, 4, or 5 minutes); and (ii) cooling the solution at a rate of about 0.1° C. to about 2° C. per second to a temperature used for the ligation (e.g. about 30° C. to about 40° C.).

In some embodiments, the ligation reaction is performed according to a method described herein. In some embodiments, the ligation is carried out at about 15° C. to about 45° C., or about 30° C., 31° C., 32° C., 33° C., 34° C., 35° C., 36° C., 37° C., 38° C., 39° C., or 40° C. In some embodiments, the ligation is carried out for about 0.1 to about 48 hours, or about 10, 11, 12, 13, 14, 15, 16, 17, 18, 19, or 20 hours. In some embodiments, the ligation is performed using a protease or a chelating agent. In some embodiments, the ligation is performed using a crowding agent. In some embodiments, the ligation proceeds to at least about 10%, 20%, 30%, 40%, 50%, 60%, 70%, 80%, or 90% completion. In some embodiments, the sgRNA is purified following synthesis, e.g., using a chromatographic method.

### EXAMPLES

The practice of the present invention will employ, unless otherwise indicated, conventional techniques of molecular biology, microbiology, cell biology, biochemistry, nucleic acid chemistry, and immunology, which are known to those skilled in the art. Non-limiting examples include Sambrook & Russell (2012) Molecular Cloning: A Laboratory Manual (4th ed.); Ausubel (1987) Current Protocols in Molecular Biology, New York, N.Y.: Wiley (including supplements through 2014); Bollag et al. (1996) Protein Methods. New York, N.Y.: Wiley-Liss; Huang et al. (2005) Nonviral Vectors for Gene Therapy. San Diego: Academic Press; Kaplittet al. (1995) Viral Vectors: Gene Therapy and Neuroscience Applications. San Diego, Calif.: Academic Press; Lefkovits (1997) The Immunology Methods Manual: The Comprehensive Sourcebook of Techniques. San Diego, Calif.: Academic Press; Doyle et al. (1998) Cell and Tissue Culture: Laboratory Procedures in Biotechnology. New York, N.Y.: Wiley; Mullis, Ferre & Gibbs (1994) PCR: The Polymerase Chain Reaction. Boston: Birkhauser Publisher; Greenfield (2014) Antibodies: A Laboratory Manual (2nd ed.). New York, N.Y.: Cold Spring Harbor Laboratory Press; Beaucage et al. (2000) Current Protocols in Nucleic Acid Chemistry. New York, N.Y.: Wiley, (including supplements through 2014); and Makrides (2003) Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1: Splint-Mediated Ligation of an Exemplary gRNA

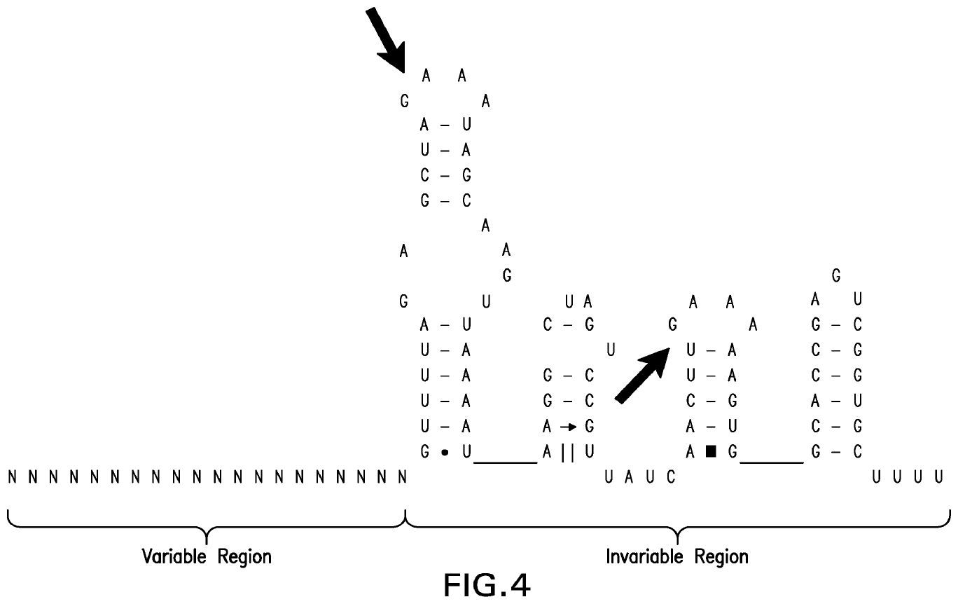

To demonstrate that RNA fragments synthesized chemically can be used to create full-length gRNA products using splint-mediated ligation, an exemplary gRNA molecule that targets a single locus in the mouse genome was synthesized using this method. The gRNA was split into three RNA fragments each having a length of under 40 nucleotides (FIG. 4). The sequences of the three RNA fragments and the two DNA splint oligonucleotides are shown in Table 1. The locations to segment the gRNA were selected to remove internal hairpins that would interfere with hybridization with a DNA splint oligonucleotide. Because these locations for segmenting the gRNA were in the invariable region of the gRNA (i.e., downstream of the spacer or the variable region), these locations can be used for any gRNA used by Cas enzymes derived from Streptococcus pyogenes (SpyCas or SpCas), such as SpyFi, and only the RNA at the terminal 5′ position of the gRNA construct (e.g., RNA1 in Table 1), and its corresponding splint (e.g., Splint1->2 in Table 1), need to be uniquely synthesized for different genomic targets.

TABLE 1
Exemplary sequences of RNA fragments and DNA
splint oligonucleotides

| | # of | |
|---|---|---|
| | nucleo- | |
| Oligos | tides | Sequence |
| RNA 1 | 33 | GAGAACGCACCACUUUACGAGUUUUAGAGCUAG |
| | | (SEQ ID NO: 2) |
| RNA 2 | 39 | pAAAUAGCAAGUUAAAAUAAGGCUAGUCCGUUAU |
| | | CAACUU(SEQ ID NO: 3) |
| RNA 3 | 28 | pGAAAAAGUGGCACCGAGUCGGUGCUUUU (SEQ |
| | | ID NO: 4) |
| Splint | 36 | CCTTATTTTAACTTGCTATTTCTAGCTCTAAAAC |
| 1→2 | | TC (SEQ ID NO: 5) |
| Splint | 48 | AAAAGCACCGACTCGGTGCCACTTTTTCAAGTTG |
| 2→3 | | ATAACGGACTAGCC (SEQ ID NO: 6) |

Key p = phosphate

Selection Criteria for gRNA Segmentation

Under biological conditions, RNAs associated with the Cas protein family have internal structures that can interfere with their hybridization to complementary splint oligonucleotides. For example, when assembled with the Cas protein, gRNA associated with spCas9 has four stem loops (hairpins), and gRNA associated with saCas9 has three stem loops. These stem loops, particularly two tetraloops, are assumed to exist when gRNA is unassociated with the Cas protein. To minimize intramolecular association of the RNA fragments which would preclude hybridization with the splints, locations for segmentation of gRNA were selected within these stem loop motifs to disrupt these energetically favorable secondary structures. The stability of the stem loop depends on the stability of the stem/helix (i.e., the number of nucleotides forming the helix) and loop (i.e., the type of bases and number of nucleotides forming the loop) regions. Division within the loop region removes the stem loop and is thus generally preferable. Division within the helix is also compatible with this approach and free energy calculations were used to ensure appropriate binding of the RNA fragments to the splint.

### Selection of DNA Splint Oligonucleotides

The length of the DNA splint oligonucleotides were selected to promote duplex formation between the splints and RNA fragments over formation of intramolecular structures within the RNA fragments. This was accomplished in silico by comparing the energetics of internal structures to the energetics of duplexes formed between the splint and pre-ligated RNA fragments. Minimum free energy prediction algorithms (mFold) were used to calculate the free energy of the RNA secondary structure(s) (ΔGᵢₙₜᵣₐ) of an individual fragment and the intermolecular hybridization between the fragment and the splint (ΔGᵢₙₜₑᵣ). The length of the splint was extended until the criteria listed below were met to ensure favorable binding of the fragment to the splint during ligation.

The free energy of the intramolecular structure was set to be greater than the free energy of the intermolecular structure, which is inversely proportionate to their melting temperatures (Tₘ).

ΔGᵢₙₜᵣₐ>ΔGᵢₙₜₑᵣ˜Tₘ₋ᵢₙₜᵣₐ<Tₘ₋ᵢₙₜₑᵣ

In addition, the temperature at which the ligation reaction was performed (Tᵣₓₙ) was set to be lower than the Tₘ of the RNA/DNA splint complex.

Tᵣₓₙ<Tₘ₋ᵢₙₜₑᵣ

DNA splint oligonucleotides were used because i) T4 RNA Ligase II can use DNA/RNA heteroduplexes for ligation, and ii) DNA is easier and less expensive to make than RNA. Splints can, however, also be synthesized from RNA, non-natural nucleic acids, artificial nucleic acids (e.g., peptide nucleic acids), or any nucleic acid mimetic. The program used to determine free energy and stability can be accessed from URL unafold.rna.albany.edu/?q=mfold

### Chemical Synthesis and Purification of RNA Fragments

RNA fragments and DNA splint oligonucleotides were synthesized using standard phosphoramidite chemistry with extension in the 3′ to 5′ direction. Since one of the substrates for T4 RNA ligase is a 5′ phosphorylated RNA oligomer, all RNA fragments except the fragment that will become the terminal 5′ end of the gRNA (e.g., RNA 1 in Table 1) were synthesized with a terminal 5′ phosphate. All RNA fragments were purified by reversed-phase HPLC, ion-exchange chromatography, or PAGE, before use. Since phosphorylation is the final coupling step during the synthesis of these RNAs, truncation products from their synthesis will not be incorporated during enzymatic ligation. The only truncation products that can be incorporated will come from the 5′ terminal fragment. For this reason, the 5′ terminal fragment was purified before ligation and it is advantageous to design this fragment to be less than 40 nucleotides. In some cases, the 5′ terminal fragment is synthesized in the 5′ to 3′ direction, while the other fragments are synthesized in the 3′ to 5′ direction, which would prevent truncation products from the synthesis of the 5′ terminal fragment from being included in the final ligation product.

### Modified Oligonucleotides

RNA fragments with non-natural (modified) ribophosphate backbones can also be used to synthesize gRNA using splint-mediated ligation as described herein. As an example, ligation of RNA fragments with a methoxy substitution of the 2′ hydroxyl group at the 3′ terminal ligation site or one to two nucleotides away from the 3′ terminal ligation site was achieved. These RNA fragments also contained phosphorothioate linkages that were 20 or more nucleotides from the ligation site. These modifications may also be tolerated at or near the ligation site.

### Ligation Reaction

The RNA fragments and DNA splints were combined in a T4 RNA Ligase II reaction buffer (New England Biolabs (NEB)) at a concentration of 10 or 20 μM each. The concentrations of the RNA fragments and DNA splints were substantially equal. The solution was heated to 90° C. for 3 minutes and cooled at a rate of 1° C./second to 37° C. to disrupt the internal structures of the RNA fragments and allow annealing of the DNA splints and the RNA fragments. T4 RNA Ligase II was then added to the solution followed by incubation at 37° C. for 0.5 to 24 hours. As defined by NEB, one unit of T4 RNA Ligase II is the amount of enzyme required to ligate 0.4 μg of an equimolar mix of a 23-mer and a 17-mer RNA in a total reaction volume of 20 μL in 30 minutes at 37° C. The ligation reaction can also work at other temperatures, e.g. 25° C., the reaction time can be increased, and the reaction can be quenched using a protease or EDTA. A crowding agent (e.g., PEG) can be added to the ligation reaction.

### Isolation of Full-Length Product

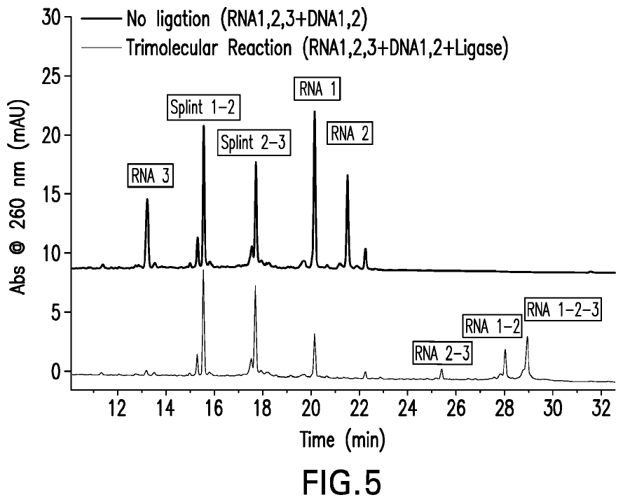

The full-length gRNA product was isolated from the DNA splint oligonucleotides and un-ligated RNA 1, 2 or 3 fragments using ion exchange high-performance liquid chromatography (HPLC). FIG. 5 is an HPLC chromatogram showing the presence of oligonucleotides before (top trace) and after (bottom trace) the ligation reaction. The presence of ligation products RNA2-3, RNA1-2 and the full-length gRNA (RNA 1-2-3) were detected.

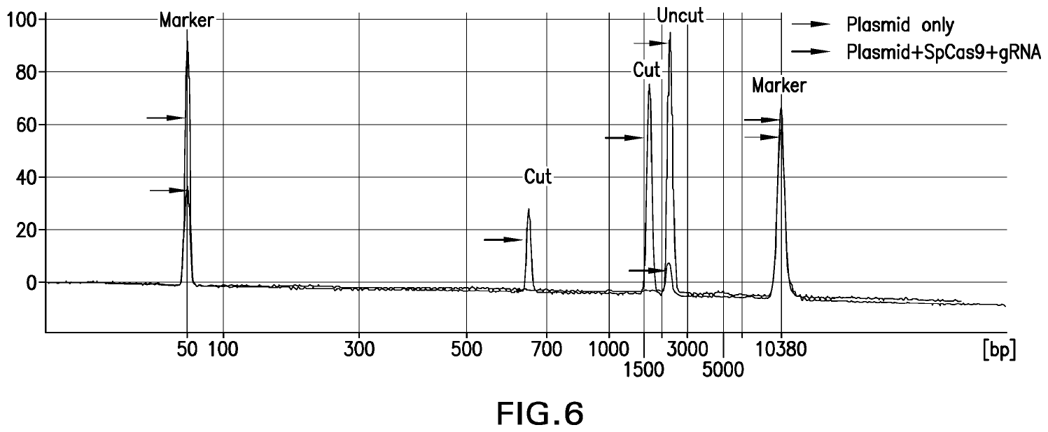

The full-length gRNA product was tested in combination with SpCas9 using a plasmid containing the target DNA sequence. As shown in FIG. 6, the plasmid was cleaved by SpCas9 at the appropriate target site.

These results demonstrated that RNA fragments synthesized chemically can be used to create full-length gRNA products using splint-mediated ligation.

### Example 2: Splint-Mediated Ligation of Modified RNAs

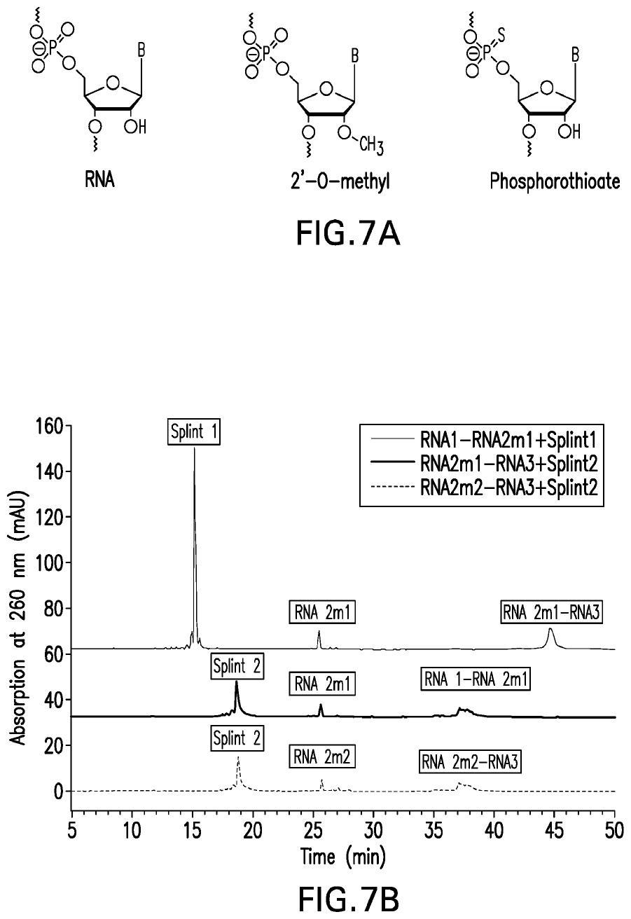

To examine whether splint-mediated ligation could be used to ligate RNA fragments containing modifications, exemplary modified RNAs (RNA 2m1 and RNA 2m2) as shown in Table 2 below were ligated using DNA splint oligonucleotides, Splint 1 and Splint 2. FIG. 7A shows the chemical structures for the unmodified RNA and exemplary modifications.

TABLE 2
Sequences of Modified RNA Fragments and Splint
Oligonucleotides

| Name | Sequence |
|---|---|
| RNA 1 | mGmsAmsGAACGCACCACUUUACGAGUUUUAGAmGmCmUmAG |
| | (SEQ ID NO: 7) |
| RNA | pAAAmUmAmGmCAAGUUAAAAUAAGGCUAGUCCGUUAUCmAm |
| 2m1 | AmCmUmU (SEQ ID NO: 8) |
| RNA | pAAAmUmAmGmCAAGUUAAAAUAAGGCUAGUCCGUUAUCmAm |
| 2m2 | AmCUU (SEQ ID NO: 9) |
| RNA 3 | pGAAAmAmAmGmUmGmGmCmAmCmCmGmAmGmUmCmGmGmUm |
| | GmCmUmsUmsUmsU (SEQ ID NO: 10) |
| Splint | CCTTATTTTAACTTGCTATTTCTAGCTCTAAAACTC |
| 1 | (SEQ ID NO: 5) |
| Splint | AAAAGCACCGACTCGGTGCCACTTTTTCAAGTTGATAACGGA |
| 2 | CCTAGC (SEQ ID NO: 6) |

Key p = phosphate; mX = 2′-O-methyl; msX = 2′-O-methyl with phosphorothioate

FIG. 7B is a chromatogram showing results from HPLC analyses of the ligation products. Ligation products RNA2m1-RNA3, RNA1-RNA2m1, and RNA2m2-RNA3 were detected.

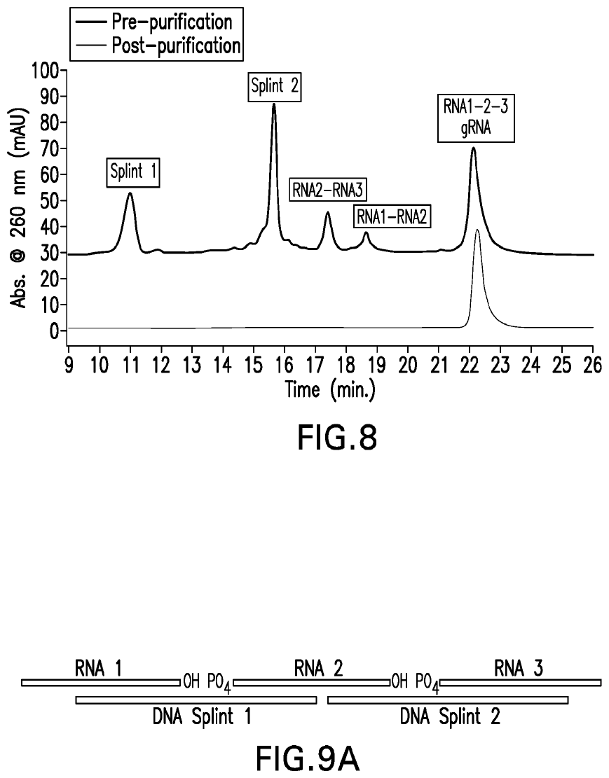

Next, RNA1, RNA2m1 (shown as RNA2 in Table 3 below and in FIG. 8), RNA3 were ligated to generate the full-length gRNA using Splint 1 and Splint 2.

TABLE 3
Sequences of Modified RNA Fragments and Splint
Oligonucleotides

| Name | Sequence |
|---|---|
| RNA 1 | mGmsAmsGAACGCACCACUUUACGAGUUUUAGAmGmCmUmAG |
| | (SEQ ID NO: 7) |
| RNA 2 | pAAAmUmAmGmCAAGUUAAAAUAAGGCUAGUCCGUUAUCmAm |
| | AmCmUmU( SEQ ID NO: 8) |
| RNA 3 | pGAAAmAmAmGmUmGmGmCmAmCmCmGmAmGmUmCmGmGmUm |
| | GmCmUmsUmsUmsU (SEQ ID NO: 10) |
| Splint | CCTTATTTTAACTTGCTATTTCTAGCTCTAAAACTC |
| 1 | (SEQ ID NO: 5) |
| Splint | AAAAGCACCGACTCGGTGCCACTTTTTCAAGTTGATAACGGA |
| 2 | CTAGCC (SEQ ID NO: 6) |

Key p = phosphate; mX = 2′-O-methyl; msX = 2′-O-methyl with phosphorothioate

The full-length gRNA product was isolated from the DNA splint oligonucleotides and partial ligation products (RNA2-RNA3 and RNA1-RNA2) using HPLC. FIG. 8 is a chromatogram showing results from the HPLC analyses of the ligation products before and after purification. The full length RNA product (RNA1-2-3 gRNA) was detected and the purified full length product is shown in the bottom trace. These results indicate that RNA fragments with methylation modifications around the ligation site can be ligated using splint-mediate ligation described in the disclosure.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### Example 3: Evaluation of Reaction Conditions for the Splint-Mediated Ligation

Experimental conditions were compared for performing the splint-mediated ligation reaction of modified RNAs. This included evaluating use of (i) addition of magnesium salt to the reaction; and (ii) a heat annealing step prior to performing the ligation reaction.

The splint-mediated ligation was designed to prepare a final modified sgRNA product having a spacer sequence that targets exon 2 of the human G6PC gene, and a backbone suitable for use with SpyCas9. The sequences of the modified sgRNA product, and its corresponding unmodified version, are shown in Table 4. The splint-mediated ligation used three RNA fragments and two DNA splint oligonucleotides, each as shown in Table 4. The RNA fragments included:
(i) a 33 mer RNA 1 (SEQ ID NO: 11), which in the 5′ to 3′ direction includes: the spacer sequence, the crRNA repeat sequence of the final sgRNA product, and a first guanine of the tetraloop of the final sgRNA product;
(ii) a 39 mer RNA 2 (SEQ ID NO: 12), which in the 5′ to 3′ direction includes: a “AAA” of the tetraloop, and a 5′ segment of the tracrRNA of the final sgRNA product; and
(iii) a 28 mer RNA 3 (SEQ ID NO: 13), which includes the 3′ segment of the tracrRNA of the final sgRNA product.

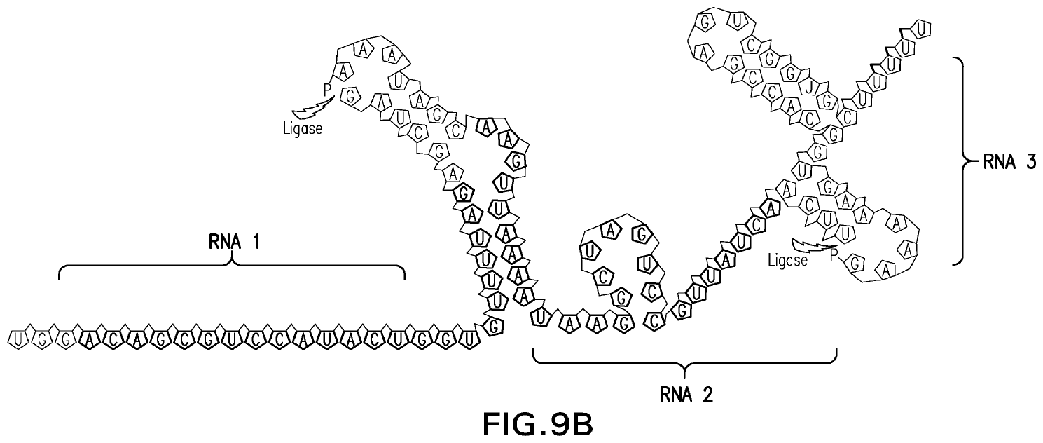

As shown in FIG. 9A, the DNA splint 1 oligonucleotide (SEQ ID NO: 5) was designed with a segment complementary to a 3′ segment of RNA 1 and a 5′ segment of RNA 2. Specifically, the sequence 5′-CTAGCTCTAAAACTC-3′ (SEQ ID NO: 22) of DNA splint 1 is complementary to the sequence 5′-GUGUUUUAGAGCUAG-3′ (SEQ ID NO: 23) of RNA 1; and the sequence 5′-CCTTATTTTAACTTGCTATTT-3′ (SEQ ID NO: 24) of DNA splint 1 is complementary to the sequence 5′-AAAUAGCAAGUUAAAAUAAGG-3′ (SEQ ID NO: 25) in RNA 2.

Additionally, the DNA splint 2 oligonucleotide (SEQ ID NO: 6) was designed with a segment complementary to a 3′ segment of RNA 2 and a 5′ segment of RNA 3. Specifically, the sequence 5′-AAGTTGATAACGGACTAG-3′ (SEQ ID NO: 26) of DNA splint 2 is complementary to the sequence 5′-CUAGUCCGUUAUCAACUU-3′ (SEQ ID NO: 27) of RNA 2; and the sequence 5′-AAAAGCACCGACTCGGTGCCACTTTTTC-3′ (SEQ ID NO: 28) in DNA splint 2 is complementary to the sequence 5′-GAAAAAGUGGCACCGAGUCGGUGCUUUU-3′ (SEQ ID NO: 29) of RNA 3.

As shown in FIG. 9B, the ligation of the RNA fragments occurs at a first ligation site between RNA fragment 1 and RNA fragment 2 that is located in the GAAA tetraloop of the repeat-anti-repeat stem loop formed between the crRNA and tracrRNA; and at a second ligation site between RNA fragment 2 and RNA fragment 3 adjacent the GAAA tetraloop of a second stem-loop in the tracrRNA. The DNA splint 1 oligonucleotide set forth in SEQ ID NO: 5 is complementary with a segment of RNA 1, however with one mismatch. The DNA splint 1 oligonucleotide set forth in SEQ ID NO: 14 is complementary with the same portion of RNA 1, but with no mismatches, and is also suitable for use in the ligation reaction.

TABLE 4
Unmodified and Modified Sequences of RNA Fragments and Splint Oligonucleotides
for Generating a G6PC-targeting sgRNA

| Sequence | | SEQ | |
|---|---|---|---|
| Name | Length | ID NO | Sequence |
| Spacer | 20 | 18 | UGGACAGCGUCCAUACUGGU |
| sequence | | 21 | msUmsGmsGACAGCGUCCAUACUGGU |
| RNA 1 | 33 nt | 15 | UGGACAGCGUCCAUACUGGUGUUUUAGAGCUAG |
| | | 11 | msUmsGmsGACAGCGUCCAUACUGGUGUUUUAGAmGmCmUmAmG |
| RNA 2 | 39 nt | 3 | pAAAUAGCAAGUUAAAAUAAGGCUAGUCCGUUAUCAACUU |
| | | 12 | pmAmAmAmUmAmGmCAAGUUAAAAUAAGGCUAGUCCGUUAUCAmAmCm |
| | | | UmU |
| RNA 3 | 28 nt | 4 | pGAAAAAGUGGCACCGAGUCGGUGCUUUU |
| | | 13 | pmGmAmAmAmAmAmGmUmGmGmCmAmCmCmGmAmGmUmCmGmGmUmGm |
| | | | CmsUmsUmsUmU |
| Splint 1 | 36 nt | 5 | CCTTATTTTAACTTGCTATTTCTAGCTCTAAAACTC |
| Splint 1 | 36 nt | 14 | CCTTATTTTAACTTGCTATTTCTAGCTCTAAAACAC |
| Splint 2 | 48 nt | 6 | AAAAGCACCGACTCGGTGCCACTTTTTCAAGTTGATAACGGACTAGCC |
| sgRNA | 100 nt | 19 | UGGACAGCGUCCAUACUGGUGUUUUAGAGCUAGAAAUAGCAAGUUAAA |
| ligation | | | AUAAGGCUAGUCCGUUAUCAACUUGAAAAAGUGGCACCGAGUCGGUGC |
| product | | | UUUU |
| | | 20 | msUmsGmsGACAGCGUCCAUACUGGUGUUUUAGAmGmCmUmAmGmAmA |
| | | | mAmUmAmGmCAAGUUAAAAUAAGGCUAGUCCGUUAUCAmAmCmUmUmG |
| | | | mAmAmAmAmAmGmUmGmGmCmAmCmCmGmAmGmUmCmGmGmUmGmCms |
| | | | UmsUmsUmU |

Key p = phosphate; mX = 2′-O-methyl; msX = 2′-O-methyl with phosphorothioate

Each oligonucleotide was dissolved in water at a concentration of 1 mM. An equal molar ratio mixture of the RNA fragments and DNA splints was prepared. The RNA/DNA mixture was then heated to 90° C. for 3 minutes and cooled at a rate of 1° C./second to 37° C. to disrupt the internal structures of the RNA fragments and allow annealing to form the DNA/RNA hybrid structures (“with annealing”). Alternatively, this step was skipped (“no annealing step”).

The RNA/DNA mixture, either with annealing or with no annealing, was then diluted in a T4 RNA Ligase II reaction buffer (New England Biolabs (NEB)) with 50 U T4 RNA Ligase II at a final concentration of 10 μM per each RNA and DNA splint oligonucleotide. The ligation reaction was either prepared by addition of MgCl₂ to provide a final concentration of 12.5 mM, or with no addition of MgCl₂. The solution was incubated at 37° C. for 16 hours. The reaction was stopped either by addition of Proteinase K, or by quenching with EDTA. The reaction mixtures were then analyzed by ion exchange HPLC for presence of full-length gRNA product and partial ligation products (RNA2-RNA3 and RNA1-RNA2).

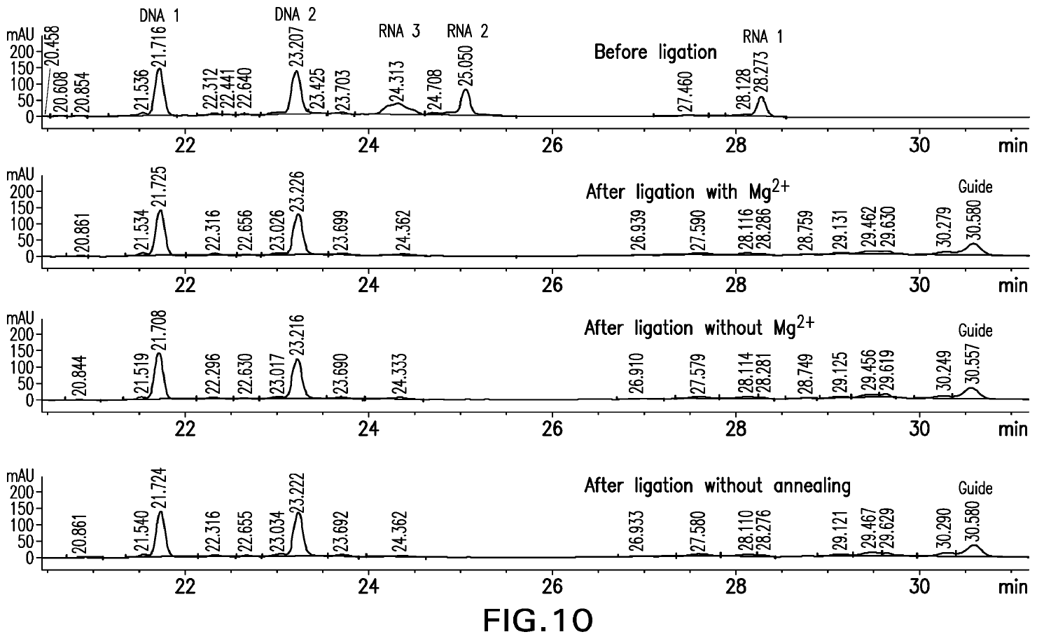

As shown in FIG. 10, the full-length RNA product (RNA1-2-3 gRNA; retention time 30.58 min) was detected in the ligation reaction for each condition evaluated. The results indicate the splint-mediated ligation reaction of modified RNA fragments is achieved without an initial annealing step, and can be successfully performed in the presence or absence of added magnesium salt.

### Example 4: Design Variations to Achieve a Splint-Mediated Ligation Reaction

Additional designs were developed to prepare the final sgRNA product (modified sequence set forth in SEQ ID NO: 20) described in Example 3 using a splint-mediated ligation reaction. The designs are based on ligation of three RNA fragments using two splint oligonucleotides. One distinction in the designs is the extent of complementarity between the first splint oligonucleotide and the first RNA fragment. As described further below, the first RNA fragment includes the variable spacer sequence and a portion of the invariable sequence of the final sgRNA product. The first design has a first splint oligonucleotide which is complementary only to the invariable segment of the first RNA fragment, and the second design has a first splint oligonucleotide which is complementary with both the invariable segment and a portion of the variable segment. As a result, the first design provides a set of components wherein only the first RNA fragment is altered to prepare sgRNAs with different target specificity. Based on the second design, both the first RNA fragment and the first splint oligonucleotide are altered to prepare sgRNAs with different target specificity. However, an advantage of this design is the increased overlap of the first splint oligonucleotide and the first RNA fragment is expected to increase the stability (i.e., melting temperature) of the RNA/DNA heteroduplex formed between these components, thus promoting heteroduplex formation at the temperature used for the ligation reaction (e.g. 37° C.).

The first design includes the RNA fragments shown in Table 5, shown as either modified or unmodified versions. Specifically, the RNA fragments include:
(i) a 34 mer RNA 1, which 5′ to 3′ incorporates the spacer sequence (SEQ ID NO: 20), the crRNA repeat sequence, and a portion of the tetraloop of the repeat-anti-repeat stem loop of the final sgRNA;
(ii) a 34 mer RNA 2, which includes the remaining portion of the tetraloop of the repeat-anti-repeat stem loop, the tracrRNA anti-repeat sequence, and a portion of the tracrRNA extending to the base of a second stem loop in the tracrRNA; and
(iii) a 32 mer RNA 3, which includes the remaining 3′ portion of the tracrRNA.

The DNA splint 1 oligonucleotide (SEQ ID NO: 44) is designed with a segment that is complementary to a 3′ segment of RNA 1 and a 5′ segment of RNA 2. Specifically, the sequence 5′-TCTAGCTCTAAAAC-3′ (SEQ ID NO: 30) of DNA splint 1 is complementary to the sequence 5′-GUUUUAGAGCUAGA-3′ (SEQ ID NO: 31) of RNA 1; and the sequence 5′-TTATTTTAACTTGCTATT-3′ (SEQ ID NO: 32) of DNA splint 1 is complementary to the sequence 5′-AAUAGCAAGUUAAAAUAA-3′ (SEQ ID NO: 33) of RNA 2.

Additionally, the DNA splint 2 oligonucleotide (SEQ ID NO: 45) is designed with a segment that is complementary to a 3′ segment of RNA 2 and a 5′ segment of RNA 3. Specifically, the sequence 5′-TGATAACGGACTAGCC-3′ (SEQ ID NO: 34) of DNA splint 2 is complementary to the sequence 5′-GGCUAGUCCGUUAUCA-3′ (SEQ ID NO: 35) of RNA 2; and the sequence 5′-TCGGTGCCACTTTTTCAAGT-3′ (SEQ ID NO: 36) of DNA splint 2 is complementary to the sequence 5′-ACUUGAAAAAGUGGCACCGA-3′ (SEQ ID NO: 37) of RNA 3.

A benefit of this design is the first splint oligonucleotide (DNA splint 1) is complementary to a segment of the first RNA fragment (RNA 1) that does not include the spacer sequence. Thus, the remaining first and second splint oligonucleotide (DNA splint 1 and DNA splint 2), and the second and third RNA fragment (RNA 2 and RNA 3) are “universal,” in that they are used to prepare sgRNA for use with SpyCas9 that has the invariable backbone sequence set forth in SEQ ID NO: 17. Only the first RNA fragment, which includes the spacer sequence of the final sgRNA, is customized depending on the desired targeting specificity of the sgRNA.

TABLE 5
Unmodified and Modified Sequences of RNA Fragments
and Splint Oligonucleotides for Generating a G6PC-
targeting sgRNA

| | | SEQ | |
|---|---|---|---|
| Sequence | | ID | |
| Name | Length | NO | Sequence |
| RNA 1 | 34 nt | 38 | UGGACAGCGUCCAUACUGGUGUUUUAGAG |
| | | | CUAGA |
| | | 39 | msUmsGmsGACAGCGUCCAUACUGGUGUU |
| | | | UUAGAmGmCmUmAmGmA |
| RNA 2 | 34 nt | 40 | pAAUAGCAAGUUAAAAUAAGGCUAGUCCG |
| | | | UUAUCA |
| | | 41 | pmAmAmUmAmGmCAAGUUAAAAUAAGGCU |
| | | | AGUCCGUUAUCA |
| RNA 3 | 32 nt | 42 | pACUUGAAAAAGUGGCACCGAGUCGGUGC |
| | | | UUUU |
| | | 43 | pmAmCmUmUmGmAmAmAmAmAmGmUmGmG |
| | | | mCmAmCmCmGmAmGmUmCmGmGmUmGmCm |
| | | | sUmsUmsUmU |
| Splint 1 | 32 nt | 44 | TTATTTTAACTTGCTATTTCTAGCTCTAA |
| Splint 2 | 36 nt | 45 | AACTCGGTGCCACTTTTTCAAGTTGATAA |
| | | | CGGACTAGCC |

Key p = phosphate; mX = 2′-O-methyl; msX = 2′-O-methyl with phosphorothioate

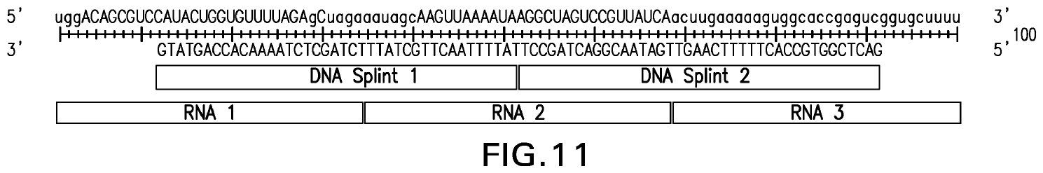

The second design includes the RNA fragments shown in Table 6, shown as either modified or unmodified versions, and which are identical to those used in the first design described above. A schematic of the second design is represented in FIG. 11, which provides a sequence alignment of the first, second, and third RNA fragments relative to the first and second DNA splints. The DNA version of the nucleotide sequence of the final sgRNA product is shown in the 5′ to 3′ direction (RNA version of the nucleotide sequence is set forth in SEQ ID NO: 19), and the segments of the nucleotide sequence that correspond to the first, second, and third RNA fragment are indicated (RNA version of the first, second, and third RNA fragments are set forth in SEQ ID NOs: 38, 40, and 42 respectively). Also shown is the alignment of the first and second DNA splint with the first, second, and third RNA fragments to form an RNA/DNA duplex, with the nucleotide sequences of the first and second DNA splints shown in the 3′ to 5′ direction (nucleotide sequences set forth in SEQ ID NOs 52 and 53 respectively).

The DNA splint 1 oligonucleotide (SEQ ID NO: 52) of the second design has a segment complementary to a 3′ segment of RNA 1 and a 5′ segment of RNA 2. Specifically, the sequence 5′-TCTAGCTCTAAAACACCAGTATG-3′ (SEQ ID NO: 46) of DNA splint 1 is complementary to the sequence 5′-CAUACUGGUGUUUUAGAGCUAGA-3′ (SEQ ID NO: 47) of RNA 1; and the sequence 5′-TATTTTAACTTGCTATT-3′ (SEQ ID NO: 48) of DNA splint 1 is complementary to the sequence 5′-AAUAGCAAGUUAAAAUA-3′ (SEQ ID NO: 49) of RNA 2. According to this design, the DNA splint 1 overlaps with 9 nucleotides in RNA 1 that are present at the 3′end of the spacer sequence. The extended overlap with the first RNA fragment is expected to increase stability of the heteroduplex formed between the first splint oligonucleotide and the first RNA fragment, specifically increasing the melting temperature of the heteroduplex, and promoting heteroduplex formation under conditions used for the ligation reaction.

Additionally, the DNA splint 2 oligonucleotide (SEQ ID NO: 53) is designed with a segment complementary to the 3′ portion of RNA 2 and a 5′ portion of RNA 3. Specifically, the sequence 5′-TGATAACGGACTAGCCT-3′ (SEQ ID NO: 50) of DNA splint 2 is complementary to the sequence 5′-AGGCUAGUCCGUUAUCA-3′ (SEQ ID NO: 51) of RNA 2; and the sequence 5′-GACTCGGTGCCACTTTTTCAAGT-3′ (SEQ ID NO: 54) of DNA splint 2 is complementary to the sequence 5′-ACUUGAAAAAGUGGCACCGAGUC-3′ (SEQ ID NO: 55) of RNA 3.

TABLE 6
Unmodified and Modified Sequences of RNA Fragments
and Splint Oligonucleotides for Generating a G6PC-
targeting sgRNA

| | | SEQ | |
|---|---|---|---|
| Sequence | | ID | |
| Name | Length | NO | Sequence |
| RNA 1 | 34 nt | 38 | UGGACAGCGUCCAUACUGGUGUUUUAGAG |
| | | | CUAGA |
| | | 39 | msUmsGmsGACAGCGUCCAUACUGGUGUU |
| | | | UUAGAmGmCmUmAmGmA |
| RNA 2 | 34 nt | 40 | pAAUAGCAAGUUAAAAUAAGGCUAGUCCG |
| | | | UUAUCA |
| | | 41 | pmAmAmUmAmGmCAAGUUAAAAUAAGGCU |
| | | | AGUCCGUUAUCA |
| RNA 3 | 32 nt | 42 | pACUUGAAAAAGUGGCACCGAGUCGGUGC |
| | | | UUUU |
| | | 43 | pmAmCmUmUmGmAmAmAmAmAmGmUmGmG |
| | | | mCmAmCmCmGmAmGmUmCmGmGmUmGmCm |
| | | | sUmsUmsUmU |
| Splint 1 | 40 nt | 52 | TATTTTAACTTGCTATTTCTAGCTCTAAA |
| | | | ACACCAGTATG |
| Splint 2 | 40 nt | 53 | GACTCGGTGCCACTTTTTCAAGTTGATAA |
| | | | CGGACTAGCCT |

Key p = phosphate; mX = 2′-O-methyl; msX = 2′-O-methyl with phosphorothioate

### SEQUENCE LISTING

SEQUENCE LISTING

| Name/ | | SEQ ID |
|---|---|---|
| Identifier | Sequence | NO: |
| residues 47-57 of | YGRKKRRQRRR | 1 |
| HIV-1 TAT | | |
| RNA 1 | GAGAACGCACCACUUUACGAGUUUUAGAGCUAG | 2 |
| RNA 2 | pAAAUAGCAAGUUAAAAUAAGGCUAGUCCGUUAUCAACUU | 3 |
| RNA 3 | pGAAAAAGUGGCACCGAGUCGGUGCUUUU | 4 |
| Splint 1->2 | CCTTATTTTAACTTGCTATTTCTAGCTCTAAAACTC | 5 |
| Splint 2->3 | AAAAGCACCGACTCGGTGCCACTTTTTCAAGTTGATAACGGACTAGCC | 6 |
| RNA 1 | mGmsAmsGAACGCACCACUUUACGAGUUUUAGAmGmCmUmAG | 7 |
| RNA 2m1 | pAAAmUmAmGmCAAGUUAAAAUAAGGCUAGUCCGUUAUCmAmAmCmUmU | 8 |
| RNA 2m2 | pAAAmUmAmGmCAAGUUAAAAUAAGGCUAGUCCGUUAUCmAmAmCUU | 9 |
| RNA 3 | pGAAAmAmAmGmUmGmGmCmAmCmCmGmAmGmUmCmGmGmUmGmCmUmsUms | 10 |
| | UmsU | |
| RNA 1 | msUmsGmsGACAGCGUCCAUACUGGUGUUUUAGAmGmCmUmAmG | 11 |
| RNA 2 | pmAmAmAmUmAmGmCAAGUUAAAAUAAGGCUAGUCCGUUAUCAmAmCmUmU | 12 |
| RNA 3 | pmGmAmAmAmAmAmGmUmGmGmCmAmCmCmGmAmGmUmCmGmGmUmGmCmsU | 13 |
| | msUmsUmU | |
| Splint 1 alt | CCTTATTTTAACTTGCTATTTCTAGCTCTAAAACAC | 14 |
| RNA 1 | UGGACAGCGUCCAUACUGGUGUUUUAGAGCUAG | 15 |
| SpyCas9 sgRNA | N₁₅₋₃₀ | 16 |
| | GUUUUAGAGCUAGAAAUAGCAAGUUAAAAUAAGGCUAGUCCGUUAUCAACUU | |
| | GAAAAAGUGGCACCGAGUCGGUGCUUUU | |
| SpyCas9 sgRNA | GUUUUAGAGCUAGAAAUAGCAAGUUAAAAUAAGGCUAGUCCGUUAUCAACUU | 17 |
| backbone | GAAAAAGUGGCACCGAGUCGGUGCUUUU | |
| G6PC Spacer | UGGACAGCGUCCAUACUGGU | 18 |
| Sequence | | |
| unmodified | | |
| Unmodified G6PC | UGGACAGCGUCCAUACUGGUGUUUUAGAGCUAGAAAUAGCAAGUUAAAAUAA | 19 |
| sgRNA | GGCUAGUCCGUUAUCAACUUGAAAAAGUGGCACCGAGUCGGUGCUUUU | |
| Modified G6PC | msUmsGmsGACAGCGUCCAUACUGGUGUUUUAGAmGmCmUmAmGmAmAmAmU | 20 |
| sgRNA | mAmGmCAAGUUAAAAUAAGGCUAGUCCGUUAUCAmAmCmUmUmGmAmAmAmA | |
| | mAmGmUmGmGmCmAmCmCmGmAmGmUmCmGmGmUmGmCmsUmsUmsUmU | |
| G6PC Spacer | msUmsGmsGACAGCGUCCAUACUGGU | 21 |
| sequence modified | | |
| DNA splint 1 | CTAGCTCTAAAACTC | 22 |
| complement to RNA | | |
| 1 | | |
| RNA 1 complement | GUGUUUUAGAGCUAG | 23 |
| to DNA splint 1 | | |
| DNA splint 1 | CCTTATTTTAACTTGCTATTT | 24 |
| complement to RNA | | |
| 2 | | |
| RNA 2 complement | AAAUAGCAAGUUAAAAUAAGG | 25 |
| to DNA splint 1 | | |
| DNA splint 2 | AAGTTGATAACGGACTAG | 26 |
| complement to RNA | | |
| 2 | | |
| RNA 2 complement | CUAGUCCGUUAUCAACUU | 27 |
| to DNA splint 2 | | |
| DNA splint 2 | AAAAGCACCGACTCGGTGCCACTTTTTC | 28 |
| complement to RNA | | |
| 3 | | |
| RNA 3 complement | GAAAAAGUGGCACCGAGUCGGUGCUUUU | 29 |
| to DNA splint 2 | | |
| DNA splint 1 | TCTAGCTCTAAAAC | 30 |
| complement to RNA | | |
| 1 | | |
| RNA 1 complement | GUUUUAGAGCUAGA | 31 |
| to DNA splint 1 | | |
| DNA splint 1 | TTATTTTAACTTGCTATT | 32 |
| complement to RNA | | |
| 2 | | |
| RNA 2 complement | AAUAGCAAGUUAAAAUAA | 33 |
| to DNA splint 1 | | |
| DNA splint 2 | TGATAACGGACTAGCC | 34 |
| complement to RNA | | |
| 2 | | |
| RNA 2 complement | GGCUAGUCCGUUAUCA | 35 |
| to DNA splint 2 | | |
| DNA splint 2 | TCGGTGCCACTTTTTCAAGT | 36 |
| complement to RNA | | |
| 3 | | |
| RNA 3 complement | ACUUGAAAAAGUGGCACCGA | 37 |
| to DNA splint 2 | | |
| Unmodified RNA 1 | UGGACAGCGUCCAUACUGGUGUUUUAGAGCUAGA | 38 |
| Modified RNA 1 | msUmsGmsGACAGCGUCCAUACUGGUGUUUUAGAmGmCmUmAmGmA | 39 |
| Unmodified RNA 2 | pAAUAGCAAGUUAAAAUAAGGCUAGUCCGUUAUCA | 40 |
| Modified RNA 2 | pmAmAmUmAmGmCAAGUUAAAAUAAGGCUAGUCCGUUAUCA | 41 |
| Unmodified RNA 3 | pACUUGAAAAAGUGGCACCGAGUCGGUGCUUUU | 42 |
| Modified RNA 3 | pmAmCmUmUmGmAmAmAmAmAmGmUmGmGmCmAmCmCmGmAmGmUmCmGmGm | 43 |
| | UmGmCmsUmsUmsUmU | |
| DNA Splint 1 | TTATTTTAACTTGCTATTTCTAGCTCTAAAAC | 44 |
| DNA Splint 2 | TCGGTGCCACTTTTTCAAGTTGATAACGGACTAGCC | 45 |
| DNA splint 1 | TCTAGCTCTAAAACACCAGTATG | 46 |
| complement to RNA | | |
| 1 | | |
| RNA 1 complement | CAUACUGGUGUUUUAGAGCUAGA | 47 |
| to DNA splint 1 | | |
| DNA splint 1 | TATTTTAACTTGCTATT | 48 |
| complement to RNA | | |
| 2 | | |
| RNA 2 complement | AAUAGCAAGUUAAAAUA | 49 |
| to DNA splint 1 | | |
| DNA splint 2 | TGATAACGGACTAGCCT | 50 |
| complement to RNA | | |
| 2 | | |
| RNA 2 complement | AGGCUAGUCCGUUAUCA | 51 |
| to DNA splint 2 | | |
| DNA Splint 1 | TATTTTAACTTGCTATTTCTAGCTCTAAAACACCAGTATG | 52 |
| DNA Splint 2 | GACTCGGTGCCACTTTTTCAAGTTGATAACGGACTAGCCT | 53 |
| DNA splint 2 | GACTCGGTGCCACTTTTTCAAGT | 54 |
| complement to RNA | | |
| 3 | | |
| RNA 3 complement | ACUUGAAAAAGUGGCACCGAGUC | 55 |
| to DNA splint 2 | | |
| RNA 1 | N₁₅₋₃₀GUUUUAGAGCUAG | 56 |
| RNA 1 | N₁₅₋₃₀GUUUUAGAGCUAGA | 57 |
| RNA 2 | pAAAUAGCAAGUUAAAAUAAGGCUAGUCCGUUAUCA | 58 |
| RNA 2 | AAUAGCAAGUUAAAAUAAGGCUAGUCCGUUAUCAACUU | 59 |
| Splint 1 | CCTTATTTTAACTTGCTATTTCTAGCTCTAAAAC | 60 |
| Splint 1 | TATTTTAACTTGCTATTTCTAGCTCTAAAAC | 61 |

p = phosphate; mX = 2′-O-methyl; msX = 2′-O-methyl with phosphorothioate

What is claimed is:

1. A method of synthesizing a guide RNA (gRNA), the method comprising:
providing a first RNA fragment comprising a terminal region comprising a 5′ phosphate moiety, and a second RNA fragment comprising a terminal region comprising a 3′ hydroxyl group, wherein the first RNA fragment, the second RNA fragment, or both, comprises at least a portion of a sequence that can bind to an RNA-guided endonuclease;
providing a splint oligonucleotide comprising a first portion complementary to the first RNA fragment at the terminal region comprising a 5′ phosphate moiety and a second portion complementary to the second RNA fragment at the terminal region comprising a 3′ hydroxyl group;
hybridizing the first RNA fragment, the second RNA fragment, and the splint oligonucleotide together to form a complex; and
ligating the first and second RNA fragments using a ligase at a ligation site present between the RNA complex, thereby synthesizing a gRNA.

2. The method of claim 1, wherein the length of the first and second RNA fragments are 10 to 90 nucleotides, each.

3. The method of claim 2, wherein the length of the second RNA fragment is 40 nucleotides or less.

4. The method of any one of claims 1-3, wherein the 5′ phosphate moiety is 5′-phosphate or 5′-phosphorothioate.

5. The method of any one of claims 1-4, wherein the ligase is a T4 DNA ligase, T4 RNA ligase I, or T4 RNA ligase II.

6. The method of any one of claims 1-5, wherein the splint oligonucleotide is a DNA or RNA oligonucleotide.

7. The method of any one of claims 1-6, wherein the length of the splint oligonucleotide is 20 to 100 nucleotides.

8. The method of any one of claims 1-7, wherein the splint oligonucleotide is attached to a solid support.

9. The method of any one of claims 1-8, wherein the length of the gRNA is 30 to 160 nucleotides.

10. The method of any one of claims 1-9, wherein the gRNA comprises a sequence that is complementary to a sequence in a target DNA.

11. The method of claim 10, wherein the target DNA is mammalian DNA.

12. The method of claim 11, wherein the target DNA is human DNA.

13. The method of any one of claims 1-12, wherein the ligation site corresponds to a site in a tetraloop portion of a stem-loop structure in the synthesized gRNA.

14. The method of any one of claims 1-12, wherein the ligation site corresponds to a site in a helix portion of a stem-loop structure in the synthesized gRNA.

15. The method of any one of claims 1-14, wherein the first RNA fragment, the second RNA fragment, or both, comprises at least one secondary structure, and wherein hybridizing the first RNA fragment, the second RNA fragment, and the splint oligonucleotide results in a lower free energy than that of the secondary structure with the lowest free energy.

16. The method of any one of claims 1-15, comprising ligating three or more RNA fragments.

17. The method of any one of claims 1-16, wherein providing the first and second RNA fragments comprises synthesizing the first and second RNA fragments through enzymatic synthesis or phosphoramidite chemistry.

18. The method of claim 17, wherein the second RNA fragment is synthesized in a 5′ to 3′ or a 3′ to 5′ direction.

19. The method of claim 17 or 18, wherein providing the first and second RNA fragments comprises purifying the first and second fragments after synthesis.

20. The method of any one of claims 1-19, wherein providing the splint oligonucleotide comprises synthesizing the splint oligonucleotide through enzymatic synthesis or phosphoramidite chemistry.

21. The method of claim 20, wherein providing the splint oligonucleotide comprises purifying the splint oligonucleotide after synthesis.

22. The method of claim 19 or 21, wherein purifying comprises purifying with a chromatographic method.

23. The method of claim 22, wherein the chromatographic method is reversed-phase HPLC, ion-exchange chromatography, size exclusion chromatography, hydrophobic interaction chromatography, affinity chromatography, or polyacrylamide gel purification, or any combination thereof.

24. The method of any one of claims 1-23, wherein the first RNA fragment, the second RNA fragment, or both, comprises at least one modification in the RNA backbone.

25. The method of claim 24, wherein the modification is selected from the group consisting of: 2′ methoxy (2′OMe), 2′ fluorine (2′fluoro), 2′-O-methoxy-ethyl (MOE), Locked Nucleic Acids (LNA), Unlocked Nucleic Acids (UNA), bridged nucleic acids, 2′deoxynucleic acids (DNA), and peptide nucleic acids (PNA).

26. The method of any one of claims 1-25, wherein the first RNA fragment, the second RNA fragment, or both, comprises at least one base modification.

27. The method of claim 26, wherein the base modification is selected from the group consisting of: 2-aminopurine, inosine, thymine, 2,6-diaminopurine, 2-pyrimidinone, and 5-methyl cytosine.

28. The method of any one of claims 1-27, wherein the first RNA fragment, the second RNA fragment, or both, comprises at least one phosphorothioate linkage.

29. The method of any one of claims 1-28, wherein hybridizing comprises hybridizing in a solution.

30. The method of claim 29, wherein a concentration of the splint oligonucleotide, a concentration of the first RNA fragment, and a concentration of the second RNA fragments in the solution are about equal.

31. The method of any one of claims 1-30, wherein ligating the first and second RNA fragments is carried out at 15° C.-45° C.

32. The method of claim 31, wherein ligating the first and second RNA fragments is carried out at about 37° C.

33. The method of any one of claims 1-32, wherein ligating the first and second RNA fragments is carried out for about 0.1 to about 48 hours.

34. The method of any one of claims 1-33, wherein ligating the first and second RNA fragments further comprises using a protease or a chelating agent.

35. The method of claim 34, wherein the chelating agent is EDTA, EGTA, or a combination of both.

36. The method of any one of claims 1-35, wherein ligating the first and second RNA fragments further comprises using one or more crowding agents.

37. The method of claim 36, wherein the one or more crowding agents comprise polyethylene glycol (PEG), Ficoll®, ethylene glycol, dextran, or any combination thereof.

38. The method of any one of claims 1-37, wherein ligating the first and second RNA fragments proceeds to at least 10% completion.

39. The method of claim 38, wherein ligating the first and second RNA fragments proceeds to at least 90% completion.

40. A method of synthesizing a guide RNA (gRNA), the method comprising providing:
(a) a first RNA fragment comprising a terminal region comprising a 3′ hydroxyl group;
(b) a second RNA fragment comprising a first terminal region comprising a 5′ phosphate moiety and a second terminal region comprising a 3′ hydroxyl group;
(c) a third RNA fragment comprising a terminal region comprising a 5′ phosphate moiety;
(d) a first splint oligonucleotide comprising (i) a first portion complementary to the terminal region comprising the 3′ hydroxyl group of the first RNA fragment; and (ii) a second portion complementary to the first terminal region comprising the 5′ phosphate moiety of the second RNA fragment;
(e) a second splint oligonucleotide comprising (i) a first portion complementary to the second terminal region comprising the 3′ hydroxyl group of the second RNA fragment; and (ii) a second portion complementary to the terminal region comprising the 5′ phosphate moiety of the third RNA fragment; and
(f) a ligase,
wherein hybridizing the first, second, and third RNA fragments and the first and second splint oligonucleotides results in formation of a complex having a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment; and
wherein the ligase results in a ligation of the first and second RNA fragments at the first ligation site, and of the second and third RNA fragments at the second ligation site, thereby synthesizing a gRNA.

41. The method of claim 40, wherein the gRNA comprises 5′ to 3′ the first RNA fragment linked to the second RNA fragment by a first phosphodiester bond, and the second RNA fragment linked to the third RNA fragment by a second phosphodiester bond.

42. The method of claim 41, wherein the first phosphodiester bond is formed between the 3′ hydroxyl group of the first RNA fragment and the 5′ phosphate group of the second RNA fragment, and wherein the second phosphodiester bond is formed between the 3′ hydroxyl group of the second RNA fragment and the 5′ phosphate group of the third RNA fragment.

43. The method of any one of claims 40-42, wherein the gRNA is a single-molecule gRNA (sgRNA).

44. The method of any one of claims 40-43, wherein the gRNA is about 30 to about 160 nucleotides in length.

45. The method of any one of claims 40-44, wherein the first ligation site corresponds to a site in a first stem-loop structure, wherein the first stem-loop structure is formed by hybridization of a minimum CRISPR repeat sequence and a minimum tracrRNA sequence in the gRNA.

46. The method of claim 45, wherein the site in the first stem-loop structure is in a tetraloop portion or in a helix portion.

47. The method of any one of claims 40-46, wherein the second ligation site corresponds to a site in a second stem-loop structure, wherein the second stem-loop structure is present in a tracrRNA sequence of the gRNA.

48. The method of claim 47, wherein the site in the second stem-loop structure is in a tetraloop portion or in a helix portion.

49. The method of any one of claims 40-48, wherein the first RNA fragment, the second RNA fragment, and/or the third RNA fragment comprise at least one secondary structure, and wherein the complex formed by hybridizing the first, second, and third RNA fragments and the first and second splint oligonucleotides has a lower free energy than that of the secondary structure with the lowest free energy.

50. A method of synthesizing a single-molecule guide RNA (sgRNA) for use with an RNA-guided endonuclease, the method comprising: providing a complex formed between a first RNA fragment, a second RNA fragment, a third RNA fragment, first splint oligonucleotide, and a second splint oligonucleotide; and a ligase, wherein
(a) the first RNA fragment comprises (i) a terminal region comprising a 3′ hydroxyl group;
(b) the second RNA fragment comprises (i) a first terminal region comprising a 5′ phosphate moiety, and (ii) a second terminal region comprising a 3′ hydroxyl group;
(c) the third RNA fragment comprises (i) a terminal region comprising a 5′ phosphate moiety;
(d) the first splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and
(e) the second splint oligonucleotide comprises (i) a first portion complementary to the second terminal region comprising the 3′ hydroxyl group of the second RNA fragment, and (ii) a second portion complementary to the terminal region comprising the 5′ phosphate moiety of the third RNA fragment,
wherein the complex is formed by hybridization of (a)(i) and (d)(i), (b)(i) and (d)(ii), (b)(ii) and (e)(i), and (c)(i) and (e)(ii),
wherein the complex has a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment,
wherein the ligase results in a ligation at the first ligation site and a ligation at the second ligation site to form a sgRNA comprising from 5′ to 3′: a spacer sequence and an invariable sequence that binds an RNA-guided endonuclease; the invariable sequence comprising a stem loop formed between a crRNA repeat sequence and a tracrRNA anti-repeat sequence, and a 3′ tracrRNA sequence comprising at least one stem-loop, thereby synthesizing the sgRNA for use with the RNA-guided endonuclease.

51. The method of claim 50, wherein first ligation site corresponds to a site in the stem loop formed between the crRNA repeat sequence and the tracrRNA anti-repeat sequence.

52. The method of claim 51, wherein the first ligation site corresponds to a site in the 5′ stem of the stem loop, in the tetraloop of the stem loop, or in the 3′ stem of the stem loop.

53. The method of any one of claims 50-53, wherein the 3′ tracrRNA sequence comprises a first stem loop, a second stem loop, and a third stem loop.

54. The method of claim 53, wherein the second ligation site corresponds to a site in the first stem loop, the second stem loop, or the third stem loop.

55. The method of claim 53 or 54, wherein the second ligation site corresponds to a site in the second stem loop, wherein the site is in the 5′ stem of the second stem loop, a site in the tetraloop of the second stem loop, or a site in the 3′ stem of the second stem loop.

56. The method of claim 53 or 54, wherein the second ligation site corresponds to a site adjacent to the 5′ base of the second stem loop or adjacent to the 3′ base of the second stem loop.

57. The method of any one of claims 50-56, wherein the first RNA fragment comprises a nucleotide sequence that is 5′ the first ligation site.

58. The method of any one of claims 50-57, where the second RNA fragment comprises a nucleotide sequence that is between the first ligation site and the second ligation site.

59. The method of any one of claims 50-58, wherein the third RNA fragment comprises a nucleotide sequence that is 3′ to the second ligation site.

60. The method of any one of claims 50-59, wherein the terminal region of (a)(i) comprises a nucleotide sequence of about 10 to about 30 nucleotides located at the 3′end of the first RNA fragment.

61. The method of claim 60, wherein the terminal region of (a)(i) comprises the spacer sequence of the sgRNA.

62. The method of claim 60, wherein the terminal region of (a)(i) does not comprise the spacer sequence of the sgRNA.

63. The method of any one of claims 50-62, wherein the first portion of (d)(i) is perfectly complementary to the terminal region of (a)(i), or has 1, 2, or 3 mismatches relative to the terminal region of (a)(i).

64. The method of any one of claims 50-63, wherein the first terminal region of (b)(i) comprises a nucleotide sequence of about 10 to about 30 nucleotides located at the 5′end of the second RNA fragment.

65. The method of any one of claims 50-64, wherein the second portion of (d)(ii) is perfectly complementary to the first terminal region of (b)(i), or has 1, 2, or 3 mismatches relative to the terminal region of (d)(ii).

66. The method of any one of claims 50-65, wherein the second terminal region of (b)(ii) comprises a nucleotide sequence of about 10 to about 30 nucleotides located at the 3′end of the second RNA fragment.

67. The method of any one of claims 50-66, wherein the first portion of (e)(i) is perfectly complementary to the second terminal region of (b)(ii), or has 1, 2, or 3 mismatches relative to the terminal region of (b)(ii).

68. The method of any one of claims 50-67, wherein the terminal region of (c)(i) comprises a nucleotide sequence of about 10 to about 40 nucleotides located at the 5′end of the third RNA fragment.

69. The method of any one of claims 50-68, wherein the second portion of (e)(ii) is perfectly complementary to the terminal region of (c)(i), or has 1, 2, or 3 mismatches relative to the terminal region of (c)(i).

70. The method of any one of claims 40-69, wherein the first RNA fragment, the second RNA fragment, and the third RNA fragment are each independently about 10 to about 90 nucleotides, about 10 to about 60 nucleotides, about 10 to about 50 nucleotides, about 10 to about 40 nucleotides, about 20 to about 40 nucleotides, about 30 to about 40 nucleotides in length.

71. The method of any one of claims 40-70, wherein the ligase is a T4 DNA ligase, T4 RNA ligase I, or T4 RNA ligase II.

72. The method of any one of claims 40-71, wherein the first splint oligonucleotide is a DNA or RNA oligonucleotide, and wherein the second splint oligonucleotide is a DNA or RNA oligonucleotide.

73. The method of any one of claims 40-72, wherein the first splint oligonucleotide and the second splint oligonucleotide are each independently about 20 to about 100 nucleotides, about 20 to about 90 nucleotides, about 20 to about 80 nucleotides, about 20 to about 70 nucleotides, about 20 to about 60 nucleotides, about 30 to about 60 nucleotides, or about 30 to about 50 nucleotides in length.

74. The method of any one of claims 40-73, wherein the gRNA or the sgRNA comprise a spacer sequence that is complementary to a sequence in a target DNA.

75. The method of claim 74, wherein the target DNA is mammalian DNA or human DNA.

76. The method of any one of claims 1-75, wherein the RNA-guided endonuclease is a small Cas nuclease or a small RNA-guided endonuclease.

77. The method of any one of claims 1-75, wherein the RNA-guided endonuclease is selected from the group consisting of: a Cas9, a Cas12, aCas13, and variants thereof.

78. The method of claim 77, wherein the RNA-guided endonuclease is a Streptococcus pyogenes Cas9 (SpyCas9) or a Staphylococcus aureus (SaCas9).

79. The method of any one of claims 1-75, wherein the RNA-guided endonuclease is a variant of Cas9, and the variant of Cas9 is selected from the group consisting of: a small Cas9, a dead Cas9 (dCas9), and a Cas9 nickase.

80. The method of any one of claims 50-75, wherein the RNA-guided endonuclease is a Streptococcus pyogenes Cas9 (SpyCas9).

81. The method of any one of claim 80, wherein the invariable sequence comprises the nucleotide sequence of SEQ ID NO: 17, or a nucleotide sequence having up to 1, 2, 3, 4, 5, 6, 7, 8, 9, or 10 nucleotide deletions, insertions, or substitutions relative to SEQ ID NO: 17.

82. The method of claim 80 or 81, wherein the first RNA fragment, the second RNA fragment, and the third RNA fragment respectively are selected from the nucleotide sequences comprising:
(a) (i) N₁₅₋₃₀GUUUUAGAGCUAG (SEQ ID NO: 56), wherein N₁₅₋₃₀ corresponds to the spacer sequence;
(ii) SEQ ID NO: 3; and
(iii) SEQ ID NO: 4;
(b) (i) N₁₅₋₃₀GUUUUAGAGCUAGA (SEQ ID NO: 57), wherein N₁₅₋₃₀ corresponds to the spacer sequence;
(ii) SEQ ID NO: 40; and
(iii) SEQ ID NO: 42;
(c) (i) N₁₅₋₃₀GUUUUAGAGCUAG (SEQ ID NO: 56), wherein N₁₅₋₃₀ corresponds to the spacer sequence;
(ii) SEQ ID NO: 58; and
(iii) SEQ ID NO: 42; or
(d) (i) N₁₅₋₃₀GUUUUAGAGCUAGA (SEQ ID NO: 57), wherein N₁₅₋₃₀ corresponds to the spacer sequence;
(ii) SEQ ID NO: 59; and
(iii) SEQ ID NO: 4.

83. The method of any one of claims 80-82, wherein the first splint oligonucleotide comprises the nucleotide sequence set forth in SEQ ID NO: 60; SEQ ID NO: 44; or SEQ ID NO: 61.

84. The method of claim 83, wherein no portion of the first splint oligonucleotide is complementary to the spacer sequence.

85. The method of claim 83, wherein the first splint oligonucleotide further comprises a 3′end having a nucleotide sequence that is complementary to the spacer sequence or to the 1, 2, 3, 4, 5, 6, 7, 8, 9, or 10 nucleotide(s) present at the 3′end of the spacer sequence.

86. The method of any one of claims 81-85, wherein the second splint oligonucleotide comprises the nucleotide sequence set forth in SEQ ID NO: 6; SEQ ID NO: 45; or SEQ ID NO: 53.

87. The method of any one of claims 40-86, wherein providing the first RNA fragment, the second RNA fragment, and the third RNA fragment comprises synthesis of the RNA fragments using enzymatic synthesis or phosphoramidite chemistry, optionally comprising purifying the RNA fragments after synthesis.

88. The method of claim 87, wherein synthesis of the RNA fragments using phosphoramidite chemistry comprises:
(i) synthesis of the first RNA fragment, synthesis of the second RNA fragment, and synthesis of the third RNA fragment in a 5′ to 3′ or in a 3′ to 5′ direction; or
(ii) synthesis of the first RNA fragment in a 5′ to 3′ or in a 3′ to 5′ direction and synthesis of the second RNA fragment and synthesis of the third RNA fragment in a 3′ to 5′ direction.

89. The method of any one of claims 40-88, wherein providing the first and second splint oligonucleotides comprises synthesis of the oligonucleotides using enzymatic synthesis or phosphoramidite chemistry, optionally comprising purifying the oligonucleotides after synthesis.

90. The method of any one of claims 40-89, wherein the first RNA fragment, the second RNA fragment, and/or the third RNA fragment, comprises at least one modification in the RNA backbone.

91. The method of claim 90, wherein the modification is selected from the group consisting of: 2′ methoxy (2′OMe), 2′ fluorine (2′fluoro), 2′-O-methoxy-ethyl (MOE), Locked Nucleic Acids (LNA), Unlocked Nucleic Acids (UNA), bridged nucleic acids, 2′deoxynucleic acids (DNA), and peptide nucleic acids (PNA).

92. The method of any one of claims 40-91, wherein the first RNA fragment, the second RNA fragment, and/or the third RNA fragment, comprises at least one base modification.

93. The method of claim 92, wherein the base modification is selected from the group consisting of: 2-aminopurine, inosine, thymine, 2,6-diaminopurine, 2-pyrimidinone, and 5-methyl cytosine.

94. The method of any one of claims 40-93, wherein the first RNA fragment, the second RNA fragment, and/or the third RNA fragment, comprises at least one phosphorothioate linkage.

95. The method of any one of claims 40-94, wherein hybridizing is performed in a solution, wherein the hybridizing is performed with or without an annealing step.

96. The method of claim 95, wherein the annealing step comprises (i) heating the solution to about 80° C. to about 95° C. for a period of time less than about 10 minutes; and (ii) cooling the solution at a rate of about 0.1° C. to about 2° C. per second to a temperature used for the ligation.

97. The method of claim 95 or 96, wherein a concentration of the first splint oligonucleotide, a concentration of the second splint oligonucleotide, a concentration of the first RNA fragment, a concentration of the second RNA fragment, and a concentration of the third RNA fragment in the solution are about equal.

98. The method of any one of claims 40-97, wherein the ligation is carried out at about 15° C. to about 45° C., or about 30° C., 31° C., 32° C., 33° C., 34° C., 35° C., 36° C., 37° C., 38° C., 39° C., or 40° C.

99. The method of any one of claims 40-98, wherein the ligation is carried out for about 0.1 to about 48 hours, or about 10, 11, 12, 13, 14, 15, 16, 17, 18, 19, or 20 hours.

100. The method of any one of claims 40-99, wherein the ligation further comprises using a protease or a chelating agent.

101. The method of claim 100, wherein the chelating agent is EDTA, EGTA, or a combination of both.

102. The method of any one of claims 40-101, wherein the ligation further comprises using one or more crowding agents.

103. The method of claim 102, wherein the one or more crowding agents comprise polyethylene glycol (PEG), Ficoll®, ethylene glycol, dextran, or any combination thereof.

104. The method of any one of claims 40-103, wherein the ligation proceeds to at least about 10%, 20%, 30%, 40%, 50%, 60%, 70%, 80%, or 90% completion.

105. The method of any one of claims 1-104, further comprising purifying the gRNA or the sgRNA after synthesis.

106. The method of claim 105, wherein purifying the gRNA or sgRNA comprises purifying using a chromatographic method.

107. The method of claim 106, wherein the chromatographic method is reversed-phase HPLC, ion-exchange chromatography, size exclusion chromatography, hydrophobic interaction chromatography, affinity chromatography, or polyacrylamide gel purification, or any combination thereof.

108. A method of generating a double-molecule gRNA comprising a crRNA and a tracrRNA, the method comprising:
providing a first RNA fragment comprising a terminal region comprising a 5′ phosphate moiety, and a second RNA fragment comprising a terminal region comprising a 3′ hydroxyl group, wherein the first RNA fragment, the second RNA fragment, or both, comprises at least a portion of a sequence that can bind to an RNA-guided endonuclease;
providing a splint oligonucleotide comprising a first portion complementary to the first RNA fragment at the terminal region comprising a 5′ phosphate moiety and a second portion complementary to the second RNA fragment at the terminal region comprising a 3′ hydroxyl group;
hybridizing the first RNA fragment, the second RNA fragment, and the splint oligonucleotide together to form a complex;
ligating the first and second RNA fragments using a ligase at a ligation site present between the RNA complex, thereby synthesizing a tracr RNA;
providing a crRNA that comprises a sequence that is complementary to a sequence in a target DNA; and
allowing the tracrRNA and crRNA to hybridize, thereby generating a double-molecule gRNA.

109. The method of claim 108, wherein providing the crRNA comprises synthesizing the crRNA through enzymatic synthesis or phosphoramidite chemistry.
